(19) United States
(12) Patent Application Publication
(10) Pub. No.: US 2021/0324417 A1
(43) Pub. Date: Oct. 21, 2021

(54) PRODUCTS AND METHODS FOR INHIBITION OF EXPRESSION OF MUTANT GARS PROTEIN

(71) Applicants: RESEARCH INSTITUTE AT NATIONWIDE CHILDREN'S HOSPITAL, Columbus, OH (US); THE JACKSON LABORATORY, Bar Harbor, ME (US)

(72) Inventors: Scott Quenton Harper, Powell, OH (US); Robert W. Burgess, Bar Harbor, ME (US)

(21) Appl. No.: 17/271,377

(22) PCT Filed: Aug. 29, 2019
(86) PCT No.: PCT/US2019/048832

§ 371 Date: Feb. 25, 2021

Related U.S. Application Data
(60) Provisional application No. 62/724,604, filed on Aug. 29, 2018.

(51) Int. Cl. C12N 15/86; C12N 15/113; C12N 9/00; A61K 38/53; A61P 25/00

(52) U.S. Cl. CPC C12N 15/86; C12N 15/1137; C12N 9/93; A61K 48/00; A61K 38/53; A61P 25/00; C12N 2750/14143; C12Y 601/01014

(57) ABSTRACT

RNA interference-based methods and products for inhibiting the expression of mutant Glycyl-tRNA Synthetase (GARS) genes are provided. Delivery vehicles such as recombinant adeno-associated viruses deliver DMAs encoding GARS microRNAs, as well as a replacement GARS gene that is resistant to knock down by the microRNAs. The methods have application in the treatment of N diseases or disorders associated with mutant GARS including, but not limited to, Charcot-Marie-Tooth Disease Type 2D (CMT2D).

### FIELD

RNA interference-based methods and products for inhibiting the expression of mutant Glycyl-tRNA Synthetase (GARS) genes are provided. Delivery vehicles such as recombinant adeno-associated viruses deliver DNAs encoding GARS microRNAs, as well as a replacement GARS gene that is resistant to knock down by the microRNAs. The methods have application in the treatment of Charcot-Marie-Tooth Disease Type 2D (CMT2D).

### INCORPORATION BY REFERENCE OF THE SEQUENCE LISTING

This application contains, as a separate part of disclosure, a Sequence Listing in computer-readable form (Filename: 53284A_SeqListing.txt; 64,937 bytes—ASCII text file created Aug. 29, 2019) which is incorporated by reference herein in its entirety.

### BACKGROUND

CMT2D, also known as distal spinal muscular atrophy V (dSMAV), is a rare, progressive, inherited axonal neuropathy caused by dominant mutations in GARS, an essential, housekeeping gene encoding glycyl-tRNA synthetase [Antonellis et al., Am. J. Hum. Genet., 72: 1293-1299 (2003)]. There is no treatment for CMT2D or any of the other more than 80 genetic forms of inherited peripheral neuropathy. To date, at least twelve individual mutations in GARS have been identified in patients with CMT2D, all of which result in single amino acid changes in different functional domains of glycyl-tRNA synthetase [Antonellis et al., supra; Abe and Hyasaka, J. Hum. Genet., 54: 310-312 (2009); James et al., Neurology, 67:1710-1712 (2006); Lee et al., J. Peripher. Nerv. Syst., 17: 418-421 (2012); Rohkamm et al., J. Neurol. Sci., 263:100-106 (2007)]. All of the disease-associated mutations of GARS are in-frame amino acid changes or small internal deletions distributed across the protein. However, the mechanisms through which mutant forms of GARS cause axon degeneration remain unclear, limiting the development of a targeted small molecule therapy.

All disease-associated GARS mutations studied to date cause impaired enzymatic activity in charging glycine onto tRNA^{Gly} in vitro and/or decreased cellular viability in yeast complementation assays, consistent with a loss-of-function effect. However, protein null alleles in mice and humans do not cause dominant neuropathy, ruling out haploinsufficiency and suggesting a dominant-negative (antimorph) mechanism. Furthermore, transgenic overexpression of wild-type GARS does not rescue the neuropathy in mouse models, suggesting that mutant forms of GARS adopt a toxic gain-of-function (neomorph) activity that the wild-type protein cannot outcompete. One proposed neomorphic mechanism involves the abnormal binding of mutant GARS to the developmental receptor neuropilin 1 (NRP1). This interaction interferes with the normal binding of vascular endothelial growth factor (VEGF), an endogenous ligand of NRP1, whose neurotrophic effects are critical for neuronal development and survival.

RNA interference (RNAi) is a mechanism of gene regulation in eukaryotic cells that researchers have worked on adapting for the treatment of various diseases. RNAi refers to post-transcriptional control of gene expression mediated by microRNAs (miRNAs). The miRNAs are small (21-25 nucleotides in length), noncoding RNAs that share sequence homology and base-pair with cognate messenger RNAs (mRNAs). The interaction between the miRNAs and mRNAs directs cellular gene silencing machinery to prevent the translation of the mRNAs. The RNAi pathway is summarized in Duan (Ed.), Section 7.3 of Chapter 7 in Muscle Gene Therapy, Springer Science+Business Media, LLC (2010). Section 7.4 mentions GARS RNAi therapy of CMT2D in mice to demonstrate proof-of-principle for RNAi therapy of dominant neuromuscular disorders.

As an understanding of natural RNAi pathways has developed, researchers have designed artificial miRNAs for use in regulating expression of target genes for treating disease. As described in Section 7.4 of Duan, supra, artificial miRNAs can be transcribed from DNA expression cassettes. The miRNA sequence specific for a target gene is transcribed along with sequences required to direct processing of the miRNA in a cell. Viral vectors such as adeno-associated virus have been used to deliver miRNAs to muscle.

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in ML. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-B1 genome is provided in Choudhury et al., Mol. Ther., 24(7): 1247-1257 (2016). Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hearty virus. It easily withstands the conditions used to inactivate adenovirus (56 to 65° C. for several hours), making cold preservation of AAV less critical. AAV may even be lyophilized. Finally, AAV-infected cells are not resistant to superinfection.

There remains a need in the art for treatments for CMT2D.

### SUMMARY

RNAi is described herein as an effective long-term treatment for dominant genetic disorders. As an example, methods and products are provided for treating any patient with a dominantly-inherited neuropathy or dominantly-inherited motor neuron disease by knocking down both wild-type and mutant forms of the involved gene(s), while also delivering an RNAi-resistant replacement gene.

As an example, methods and products are described herein for knocking down the expression of a mutant GARS gene and wild-type GARS gene in a patient. The methods utilize RNAi to knock down the expression. The methods also provide an RNAi-resistant replacement GARS gene. Use of the methods and products is indicated, for example, in preventing or treating CMT2D.

The methods deliver inhibitory RNAs that knock down the expression of both the wild-type and mutant GARS gene. The GARS inhibitory RNAs contemplated include, but are not limited to, antisense RNAs, small inhibitory RNAs (siRNAs), short hairpin RNAs (shRNAs) or artificial microRNAs (GARS miRNAs) that inhibit expression of the wild-type and mutant GARS gene.

GARS miRNAs are provided as well as polynucleotides encoding one or more of the GARS miRNAs. In some aspects, the disclosure includes nucleic acids comprising RNA-encoding and guide strand-encoding nucleotide sequences comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identity to the sequence set forth in any one of SEQ ID NOs: 1-50.

Exemplary GARS miRNAs comprise a full length miRNA antisense guide strand set out in any one of SEQ ID NOs: 1-25 or variants thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identity to the sequence set forth in any one of SEQ ID NOs: 1-25. Corresponding final processed guide strand sequences are respectively set out in SEQ ID NOs: 26-50 or variants thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identity to the sequence set forth in any one of SEQ ID NOs: 26-50. The antisense guide strand is the strand of the mature miRNA duplex that becomes the RNA component of the RNA induced silencing complex ultimately responsible for sequence-specific gene silencing. See Section 7.3 of Duan, supra.

GARS miRNAs can specifically bind to a segment of a messenger RNA (mRNA) encoded by a human GARS gene (represented by SEQ ID NO: 69 which is a human GARS cDNA), wherein the segment conserved relative to mRNA encoded by the wild-type mouse GARS gene (represented by SEQ ID NO: 70 which is a mouse GARS cDNA), and the segment does not encode sequence comprising an amino acid mutation associated with CMT2D. For example, a GARS miRNA can specifically bind a mRNA segment that is complementary to a sequence within nucleotides 136-323, 327-339, 544-590, 720-785, 996-1406, 1734-1913 or 1950-2187 of SEQ ID NO: 69. More particularly, a GARS miRNA can specifically bind a mRNA segment that is complementary to a sequence within nucleotides 996-1406 of SEQ ID NO: 69.

RNAi-resistant replacement GARS genes are provided. An “RNAi-resistant replacement GARS gene” has a nucleotide sequence the expression of which is not knocked down by the GARS miRNAs described herein but the nucleotide sequence still encodes a GARS protein that has glycl-tRNA synthetase activity. Exemplary RNAi-resistant replacement GARS genes are set out in SEQ ID NOs: 51-57.

Delivery of DNA encoding GARS inhibitory RNAs and/or RNAi-resistant replacement GARS genes can be achieved using a delivery vehicle that delivers the DNA(s) to a neuronal cell. For example, recombinant AAV (rAAV) vectors can be used to deliver DNA encoding GARS inhibitory RNA and RNAi-resistant replacement GARS genes. Other vectors (for example, other viral vectors such as lentivirus, adenovirus, retrovirus, equine-associated virus, alphavirus, pox viruses, herpes virus, polio virus, sindbis virus and vaccinia viruses) can also be used to deliver polynucleotides encoding GARS inhibitory RNAs. Thus, also provided are viral vectors encoding one or more GARS miRNAs and RNAi-resistant replacement GARS genes. When the viral vector is a rAAV, the rAAV lack AAV rep and cap genes. The rAAV can be self-complementary (sc) AAV. As another example, non-viral vectors such as lipid nanoparticles can be used for delivery.

Provided herein are rAAV, each encoding a GARS miRNA and an RNAi-resistant replacement GARS gene. Also provided are rAAV encoding one or more GARS miRNAs. A rAAV (with a single-stranded genome) encoding one or more GARS miRNAs can encode one, two, three, four, five, six, seven or eight GARS miRNAs, while a separate rAAV encodes an RNAi-resistant replacement GARS gene. A scAAV encoding one or more GARS miRNAs can encode one, two, three or four GARS miRNAs, while a separate rAAV encodes an RNAi-resistant replacement GARS gene. Also provided herein are rAAV comprising an RNAi-resistant replacement GARS gene.

Compositions are provided comprising the nucleic acids or viral vectors described herein.

Further provided are methods of preventing or inhibiting expression of the GARS gene in a cell comprising contacting the cell with a delivery vehicle (such as rAAV) encoding a GARS miRNA wherein, if the delivery vehicle is rAAV, the rAAV lacks rep and cap genes. In the methods, expression of the mutant GARS allele is inhibited by at least 10, at least 20, at least 30, at least 40, at least 50, at least 60, at least 70, at least 80, at least 90, at least 95, at least 98 percent, at least 99 percent, or 100 percent. In the methods, expression of the wild-type GARS allele is inhibited by at least 10, at least 20, at least 30, at least 40, at least 50, at least 60, at least 70, at least 80, at least 90, at least 95, at least 98 percent, at least 99 percent, or 100 percent.

Still further provided are methods of delivering DNA encoding the GARS miRNA and an RNAi-resistant replacement GARS gene to an animal in need thereof, comprising administering to the animal a delivery vehicle (such as rAAV) comprising DNA encoding the GARS miRNA and the RNAi-resistant replacement GARS gene wherein, if the delivery vehicle is rAAV, the rAAV lacks rep and cap genes. Other methods of delivering DNA encoding the GARS miRNA and an RNAi-resistant replacement GARS gene to an animal in need thereof, comprise administering to the animal a delivery vehicle (such as rAAV) comprising DNA encoding one or more GARS miRNA and a delivery vehicle (such as rAAV) comprising an RNAi-resistant replacement GARS gene wherein, if the delivery vehicle is rAAV, the rAAV lacks rep and cap genes.

Methods are also provided of preventing or treating CMT2D comprising administering a delivery vehicle (such as rAAV) comprising DNA encoding a GARS miRNA and an RNAi-resistant replacement GARS gene wherein, if the delivery vehicle is rAAV, the rAAV lacks rep and cap genes. Other methods of preventing or treating CMT2D comprise administering a delivery vehicle (such as rAAV) comprising DNA encoding one or more GARS miRNA and rAAV comprising an RNAi-resistant replacement GARS gene wherein, if the delivery vehicle is rAAV, the rAAV lacks rep and cap genes. The methods result in restoration of GARS glycyl-tRNA synthetase expression to at least 25 percent, at least 30, at least 40, at least 50, at least 60, at least 70, at least 80, at least 90, at least 95, at least 98 percent, at least 99 percent, or 100 percent or more, of normal GARS glycyl-tRNA synthetase expression in an unaffected individual.

The disclosure provides a nucleic acid comprising a nucleic acid encoding a GARS miRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-25; a nucleic acid encoding a GARS guide strand comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 26-50; or a nucleic acid encoding a GARS miRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-25 and a nucleic acid comprising an RNAi-resistant GARS gene comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 51-57.

The disclosure provides a viral vector comprising the nucleic acids described herein and/or a combination of any one or more thereof. In some aspects, the viral vector is an adeno-associated virus (AAV), adenovirus, lentivirus, retrovirus, poxvirus, baculovirus, herpes simplex virus, vaccinia virus, or a synthetic virus. In some aspects, the viral vector is an AAV. In some aspects, the AAV lacks rep and cap genes. In some aspects, the AAV is a recombinant AAV (rAAV) or a self-complementary recombinant AAV (scAAV). In some aspects, the AAV is AAV-1, AAV-2, AAV-3, AAV-4, AAV-5, AAV-6, AAV-7, AAV-8, AAV-9, AAV-10, AAV-11, AAV-12, AAV-13, AAV-anc80, and AAV rh.74. In some aspects, the AAV is AAV-9. In some aspects, the AAV is a pseudotyped AAV. In some aspects, the AAV is AAV2/8 or AAV2/9. In some aspects, expression of the nucleic acid encoding the GARS miRNA is under the control of a U6 promoter. In some aspects, expression of the RNAi-resistant replacement GARS gene is under the control of a chicken β-actin promoter.

The disclosure provides a composition comprising the nucleic acids described herein and a pharmaceutically acceptable carrier. The disclosure provides a composition comprising a viral vector comprising the nucleic acids described herein and/or a combination of any one or more thereof and a pharmaceutically acceptable carrier.

The disclosure provides a composition comprising a delivery vehicle capable of delivering agents to a neuronal cell and (a) a nucleic acid comprising an RNAi-resistant human GARS gene; (b) a nucleic acid encoding a miRNA, wherein the miRNA binds a segment of a messenger RNA (mRNA) encoded by a human GARS gene, wherein the segment is conserved relative to the wild-type mouse GARS gene, and wherein the segment does not encode sequence comprising a mutation associated with CMT2D; or a combination of the nucleic acids of (a) and (b) and, optionally, a pharmaceutically acceptable carrier. In some aspects, the nucleic acid in the composition comprises the RNAi-resistant human GARS gene comprising a polynucleotide comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence of any one of SEQ ID NOs: 51-57. In some aspects, the human GARS gene comprises the sequence of SEQ ID NO: 69, or a variant thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99%, identity to the sequence of SEQ ID NO: 69. In some aspects, the mouse GARS gene comprises the sequence of SEQ ID NO: 70, or a variant thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99%, identity to the sequence of SEQ ID NO: 70. In some aspects, the mRNA segment is complementary to a sequence within nucleotides 136-323, 327-339, 544-590, 720-785, 996-1406, 1734-1913 or 1950-2187 of a human GARS gene comprising the sequence of SEQ ID NO: 69. In some aspects, the mRNA segment is complementary to a sequence within nucleotides 996-1406 of SEQ ID NO: 69.

In some aspects, the delivery vehicle in the composition is a viral vector. In some aspects, the viral vector is an adeno-associated virus (AAV), adenovirus, lentivirus, retrovirus, poxvirus, baculovirus, herpes simplex virus, vaccinia virus, or a synthetic virus. In some aspects, the viral vector is an AAV. In some aspects, the AAV lacks rep and cap genes. In some aspects, the AAV is a recombinant AAV (rAAV) or a self-complementary recombinant AAV (scAAV). In some aspects, the AAV is or has a capsid serotype selected from the group consisting of: AAV-1, AAV-2, AAV-3, AAV-4, AAV-5, AAV-6, AAV-7, AAV-8, AAV-9, AAV-10, AAV-11, AAV-12, AAV-13, AAV-anc80, and AAV rh.74. In some aspects, the AAV is or has a capsid serotype of AAV-9. In some aspects, the AAV is a pseudotyped AAV. In some aspects the AAV is AAV2/8 or AAV2/9. In some aspects, the expression of the nucleic acid encoding the GARS miRNA is under the control of a U6 promoter. In some aspects, the expression of the RNAi-resistant replacement GARS gene is under the control of a chicken β actin promoter.

The disclosure provides methods of delivering to a neuronal cell comprising a mutant GARS gene, the method comprising administering to the neuronal cell: (a) a nucleic acid comprising a nucleic acid encoding a GARS miRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-25; a nucleic acid encoding a GARS guide strand comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 26-50; or a nucleic acid encoding a GARS miRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-25 and a nucleic acid comprising an RNAi-resistant GARS gene comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 51-57; (b) a vector comprising any one or more of the nucleic acids described herein; or (c) a composition comprising any one or more of the nucleic acids or vectors described herein.

The disclosure provides a method of treating a subject suffering from a mutant GARS gene, the method comprising administering to the subject: (a) a nucleic acid comprising a nucleic acid encoding a GARS miRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-25; a nucleic acid encoding a GARS guide strand comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 26-50; or a nucleic acid encoding a GARS miRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-25 and a nucleic acid comprising an RNAi-resistant GARS gene comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 51-57; (b) a vector comprising any one or more of the nucleic acids described herein; or (c) a composition comprising any one or more of the nucleic acids or vectors described herein.

In some aspects, the subject suffers from Charcot-Marie-Tooth Disease Type 2D (CMT2D) or Distal Hereditary Motor Neuropathy. In some aspects, the neuronal cell is a human neuronal cell. In some aspects, the subject is a mammalian subject. In some aspects the subject is a human subject.

The disclosure also provides uses of at least one nucleic acid as described herein, at least one viral vector as described herein, or a composition as described herein in making a medicament for or in treating a subject suffering from a mutant Glycyl-tRNA Synthetase (GARS) gene.

The disclosure also provides uses of at least one nucleic acid as described herein, at least one viral vector as described herein, or a composition as described herein in making a medicament for or in treating Charcot-Marie-Tooth Disease Type 2D (CMT2D) or Distal Hereditary Motor Neuropathy in a subject in need thereof.

Other features and advantages of the disclosure will become apparent from the following description of the drawing and the detailed description. It should be understood, however, that the drawing, detailed description, and the examples, while indicating embodiments of the disclosed subject matter, are given by way of illustration only, because various changes and modifications within the spirit and scope of the disclosure will become apparent from the drawing, detailed description, and the examples.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1A-B shows the ΔETAQ GARS mutation does not affect gene expression. FIG. 1A demonstrates that RNA and cDNA libraries were generated from patient fibroblasts. Subsequently, RT-PCR products spanning the mutation were subjected to deep-sequencing analysis. A cartoon representing the GARS exon7-exon8 junction is shown with the wild-type (blue) and mutant (red) PCR products indicated. Mapped sequence reads were deemed informative if they spanned the nucleotides affected by the mutation. Of the informative reads, 53.7% were from wild-type (WT) transcripts and 46.3% were from ΔETAQ GARS transcripts. FIG. 1B shows Western blot results. Western blot analyses were performed with total protein lysates from fibroblast cell lines from affected (Patient) and unaffected (Control) individuals using an anti-GARS or anti-actin antibody, as indicated. Sample names are across the top and protein size markers (kDa) are indicated on the left.

FIG. 2A-J shows the in vitro and in vivo characterization of a GARS mutation, the ΔETAQ GARS mutation. In FIG. 2A, the position and evolutionary conservation of the ΔETAQ (red) and P234KY (green) GARS mutations are shown, along with flanking amino acid residues. FIG. 2B shows that the body weight of GARS^{ΔETAQ/huEx8} mice and littermate controls was measured at 12 weeks. GARS^{(ΔETAQ/huEx8)} mice were significantly lighter, weighing 19±1.9 grams (p=0.0006, n=8) compared to GARS^{huEx8/+} controls, which weighed 27.4±4.84 grams (n=7). FIG. 2C shows that gross motor performance in GARS^{ΔETAQ/huEx8} mice was quantified using a wire hang test. While GARS^{huEx8/+} mice averaged 55±9.57 seconds before letting go, GARS^{ΔETAQ/huEx8} mice (n=8) fell after only 17.3±11.3 seconds. FIG. 2D shows that axon number in the motor branch of the femoral nerve was reduced by 21% from 551±45 axons in littermate controls to 438±92 axons in GARS^{ΔETAQ/huEx8} (n=6 mice per genotype). FIG. 2E shows that axons were also smaller in diameter, as shown in a cumulative histogram of axon diameters (p=p<0.0001, K-S test), with a complete absence of large diameter axons in GARS^{(ΔETAQ/huEx8)} mice (average diameter=1.6±0.8 μm, n=6) compared to GARS^{(+/huEx8)} littermates (3.3±2.198 μm n=6). FIG. 2F shows that these changes are evident in images of nerve cross sections. FIG. 2G shows nerve conduction velocity (NCV) was significantly reduced from 35±6.29 m/s in littermate controls to 13.5±4.1 m/s in GARS^{ΔETAQ/huEx8} mice (p=0.0002, n=6 GARS^{ΔETAQ/huEx8}, n=7 GARS^{huEx8/+}). FIG. 2H shows that neuromuscular junctions (NMJs) from the plantaris muscle showed partial innervation and denervation, scored based on the overlap between pre- and post-synaptic staining. While 98.0% of control NMJs were fully innervated, only 32.6% were fully innervated in GARS^{ΔETAQ/huEx8} mice, with 60% being partially innervated and 8.5% completely denervated. Representative images of NMJs morphology and innervation are shown (FIG. 2I-J) after labeling with antibodies against neurofilament and synaptic vesicles (green) and alpha-bungarotoxin. Differences between body weights, grip strength, conduction, axon number between genotypes were statistically evaluated using a two-way student's t test, while axon diameter was evaluated by a Kolmogorov-Smimov test. Significant difference in overall % NMJ innervation was determined by two-way ANOVA with Tukey's HSD posthoc comparisons. For all analyses *=p<0.05, **=p<0.01, **=p<0.001, **=p<0.0001 represents posthoc significance between genotypes. Values are mean±S.D. All scale bars=100 μm.

FIG. 3A-B shows mRNA and protein levels in GARS^{ΔETAQ/huEx8} mice. FIG. 3A shows chromatograms from Sanger sequencing analysis of RT-PCR products generated using cDNA isolated from sciatic nerve showing the first 34 bases of GARS exon 8 (n=3 mice per genotype). Human-specific nucleotides expressed within GARS^{+/huEx8} and GARS^{ΔETAQ/huEx8} are indicated by black arrows. The ΔETAQ mutation (the 12 bps deleted by the ΔETAQ mutation are highlighted in red) is noted by the box in the sequence above and is indicated by double sequence starting at base 13. FIG. 3B shows Western blot analysis of brain homogenates for GARS expression in the indicated mouse strains. The blot was re-probed with an anti-NeuN antibody to control for variability in protein loading.

FIG. 4A-H shows scAAV9.miΔETAQ prevents of the onset of neuropathy in GARS^{(ΔETAQ/huEx8)} mice. FIG. 4A shows that therapeutic miGARS microRNAs utilize naturally occurring RNAi biogenesis and gene silencing pathways in target cells. Each miGARS or control sequence was cloned as a DNA template downstream of a U6 promoter and then delivered to cells via plasmid transfection (in vitro) or within scAAV9 particles in vivo (depicted here). Once in the target cell nucleus, primary microRNA constructs are transcribed and then processed by the RNAses Drosha and Dicer and the nuclear export factor Exportin-5 (Exp5). The mature antisense strand (red line) incorporates into the RNA-Induced Silencing Complex (RISC) to elicit sequence-specific degradation of the mutant GARS mRNA. FIG. 4B shows that miRNAs were efficacy tested in vitro by co-transfecting HEK293 cells with individual U6-miGARS, or control, plasmids miRNA and a dual-luciferase reporter plasmid containing one of four target genes cloned into the 3′ UTR of Renilla luciferase: wild-type Human GARS, human ΔETAQ GARS, wild-type mouse GARS, or the mouse GARS gene containing the same ETAQ deletion. Target gene silencing was then determined by measuring the ratio of Renilla to Firefly luciferase. The values are reported as mean ratios ±SEM, and the significance of knockdown efficiency was analyzed using a two-way ANOVA. Also shown is the sequence of the guide strand of the lead miΔETAQ and its complementarity to both the wild-type and ΔETAQ GARS gene. The four amino acid deletion is shown in red. Base pairing between the miRNA and target genes is shown with vertical lines, with red lines indicating wobble G-U bonds present in RNA duplexes. FIG. 4C-E shows scAAV9.miΔETAQ treatment in vivo delivered by ICV injection to neonatal mice significantly prevented deficits at four weeks of age in gross motor performance quantified by the wire hang test (p=0.0001) as well as reductions in MW:BW ratios (p=0.0315) and NCVs (<0.0001), compared to untreated or vehicle-treated GARS^{(ΔETAQ/huEx8)} mice. Figure F-H shows that quantification of axon number and axon size indicated that scAAV9.miΔETAQ could partially prevent axon loss (p=0.0272) and reductions in axon diameter (p=<0.0001) compared to scAAV9.miLacZ-treated ΔETAQ mice, as shown in cross sections of the motor branch of the femoral nerve. Axon diameter was analyzed using a KS normality test while all other outcomes measures were analyzed using a two-way ANOVA with Tukey's HSD posthoc comparisons. *=p<0.05, *=p<0.01, **=p<0.001, ****=p<0.0001 represents posthoc significance between scAAV9.miΔETAQ- and scAAV9.miLacZ-treated ΔETAQ mice. Values are mean±S.D. All scale bars=100 μm. Untreated GARS^{(ΔETAQ/huEx8)} n=4 miLacZ-treated GARS^{(ΔETAQ/huEx8)} n=3, scAAV9.miΔETAQ-treated GARS^{(ΔETAQ/huEx8)} n=5, mi.LacZ-treated GARS^{(ΔETAQ/huEx8)} n=5, and scAAV9.miΔETAQ-treated GARS^{(ΔETAQ/huEx8)} n=5.

FIGS. 5A-E shows all miRNAs targeting ΔETAQ disease allele tested in vitro. FIG. 5A shows the five miRNAs hairpins originally tested targeting the ΔETAQ mutation in the human GARS gene. The guide strand is indicated in blue, and the passenger strand is in red. Gray and black arrowheads indicate the Drosha and Dicer cut sites respectively. FIG. 5B shows the first set of miRNAs tested in a dual-luciferase assay. HEK293 cells were cotransfected with a single miRNA and the dual luciferase reporter containing either wild-type or ΔETAQ human GARS cloned as the 3′UTR of Renilla luciferase. Gene silencing was determined by measuring the ratio of Renilla to Firefly luciferase and triplicate data are presented as the average mean ratio ±SEM. Based on the efficient knockdown of the human disease-allele and preservation of the wild-type allele, miEx8D12-1A was chosen as the lead candidate. FIG. 5C shows the lead candidate miEx8D12-1A was tested in an additional dual-luciferase assay against the mouse ΔETAQ mutant gene. Despite effective silencing of the human disease allele, it was unable to target the ΔETAQ mouse GARS mRNA. Triplicate data were averaged and presented as the mean±SEM. FIG. 5D shows the guide strand of the initial lead miRNA and its complementarity to the target regions of both human and mouse ΔETAQ GARS. Base pairing is indicated by vertical lines with G-U bonds shown in red. FIG. 5E shows the hairpin structures of six variants of miEx8D12-1A. Drosha and Dicer cut sites are indicated with grey and black arrowheads. The guide strand is shown in blue, with the changes to the original miR sequence indicated in red. G-U base pairing is shown with a red vertical line. In vitro testing results for these miRNAs is shown in FIG. 2A-J. The lead miRNA is marked with an asterisk and was renamed mi.ΔETAQ for all further testing.

FIG. 6A-F shows post-onset therapeutic effects of scAAV9.miΔETAQ. FIG. 6A-B shows the reduction in mutant GARS expression improves grip strength and increases body weight in early- and late-symptomatic GARS^{ΔETAQ/huEx8} mice. FIG. 6A shows that mi.ΔETAQ-treated early- and late-symptomatic GARS^{ΔETAQ/huEx8} mice exhibit enhanced grip strength and significant increases in body weight starting at ˜5 weeks post treatment. FIG. 6B shows that when evaluated at 7 weeks post treatment for primary signs of neuropathy, these data correlate with trending increases in MW:BW ratios and significant improvements in nerve conduction velocity in mi.ΔETAQ-treated late-symptomatic GARS^{ΔETAQ/huEx8} mice. In addition, FIG. 6B shows early-symptomatic GARS^{ΔETAQ/huEx8} mice treated with mi.ΔETAQ displayed significantly higher MW:BW ratios and faster nerve conduction velocity, most likely resulting from the greater number of motor axons observed in cross-sections of the motor branch of the femoral nerve, although improvement in axon diameter was not observed (FIGS. 6C-D). Prevention of axon loss was not observed in mi.ΔETAQ-treated late-symptomatic GARS^{ΔETAQ/huEx8} mice (FIG. 6E). Although both early- and late-symptomatic overall displayed significant increases in NMJ innervation (FIG. 6F). Data was analyzed using a one-way ANOVA followed by Tukey's HSD posthoc comparisons. Significant changes within axon diameter (FIG. 6E) were determined with a Kolmogorov-Smimov test. *=p<0.05, *=p<0.01, **=p<0.001, **=p<0.0001 represents posthoc significance between miLacZ-treated and scAAV9.miΔETAQ-treated GARS^{ΔETAQ/huEx8} mice. A=significant difference in fully innervated NMJs, B=significant difference in partially innervated NMJs, & C=significant difference in denervated NMJs. Late-symptomatic Cohort: MiLacZ-treated GARS^{(ΔETAQ/huEx8)} n=5-7, scAAV9.miΔETAQ-treated GARS^{(ΔETAQ/huEx8)} n=3-5, mi.LacZ-treated GARS^{(ΔETAQ/huEx8)} n=6, and scAAV9.miΔETAQ-treated GARS^{(ΔETAQ/huEx8)} n=7. Early-symptomatic Cohort: GARS^{(ΔETAQ/huEx8)} n=B-7, scAAV9.miΔETAQ-treated GARS^{(ΔETAQ/huEx8)} n=3-5, mi.LacZ-treated GARS^{(ΔETAQ/huEx8)} n=7, and scAAV9.miΔETAQ-treated GARS^{(ΔETAQ/huEx8)} n=9-11. Values are mean±S.D. All scale bars=100 μm.

FIG. 7A-C shows U6.miP278KY microRNAs can specifically knockdown P278KY mouse GARS mRNA in vitro. FIG. 7A shows hairpin structures of all pre-miRNAs targeting P278KY mouse GARS mRNA. The guide strand is shown in blue, while the passenger strand is in red. Drosha and Dicer cut sites are indicated by gray and black arrowheads, respectively. The best performing miRNA in vitro is marked with an asterisk and the name was shortened to miP278KY for further testing. FIG. 7B shows the sequence of the guide strand of the best performing miP278KY and its complementarity to both wild-type and mutant mouse GARS. The P278KY mutation is shown in red. Vertical lines indicate the base paring between the miRNA and the target genes, with weaker G-U bonds shown in red. Allele-specificity is achieved by effective base-pairing of the miRNA with the mutant allele, while also having much lower complementarity to the wild-type. FIG. 7C shows miRNAs were tested in vitro by cotransfecting HEK293 cells with a single miRNA and a dual-luciferase reporter containing either wild-type or P278KY mouse GARS cloned as the 3′UTR of Renilla luciferase. Target gene silencing was determined by measuring the ratio of Renilla to Firefly luciferase. Triplicate data were averaged and presented as the mean ratio ±SEM.

FIG. 8A-H shows reduction of mutant GARS by RNAi prevents neuropathy in GARS^{(P278KY/+)} mice. FIG. 8A shows scAAV9.miP278KY treatment by ICV delivery neonatally prevented deficits in gross motor performance quantified at four weeks-of-age by the wire hang test (p=0.0001) and FIG. 8B shows reductions in MW:BW ratios (p=0.0463) compared to untreated and vehicle-treated P278KY mice. FIG. 8C shows that nerve conductions velocities were also significantly improved (p=<0.0001) in treated P278KY mice. FIG. 8D shows that quantification of axon number within cross sections of the motor branch of the femoral nerve showed that while axon number was reduced by 17% in control-treated P278KY mice, axon counts in scAAV9.miP278KY-treated P278KY mice (589±15 axons) were similar to untreated control littermates (600±11 axons). FIG. 8E shows that in a cumulative histogram of axon diameters, scAAV9.miP278KY treatment also restored the presence of large diameter axons, with average axon diameter within control-treated P278KY mice being 1.98±4.47 μm, 2.71±3.71 μm for scAAV9.miP278KY-treated P278KY mice, and 3.84±3.74 in untreated GARS^{(+/+)}. The prevention of axon atrophy is evident in representative images of cross sections of the motor branch of the femoral nerve isolated from untreated GARS^{(+/+)} and GARS^{(P278KY/+)} mice as well as scAAV9.miP278KY-treated GARS^{(P278KY/+)} mice (FIG. 8F). Representative images of neuromuscular junction (NMJ) morphology isolated from plantaris muscle are shown (FIG. 8G) after labeling with antibodies against neurofilament and synaptic vesicles (green) and alpha-bungarotoxin (red). While over 70% of the NMJs are partially or completely denervated in control-treated GARS^{(P278KY/+)} mice by 4 weeks of age (FIG. 8H) less than 30% of NMJs show any degree of denervation scAAV9.miP278KY-treated GARS^{(P278KY/+)} mice. Ns for the all outcome measures include; untreated GARS^{(+/+)} n=5, control-treated GARS^{(+/+)} n=4, scAAV9.miP278KY-treated GARS^{(+/+)} n=8, untreated GARS^{(P278KY/+)} n=6, control-treated GARS^{(P278KY/+)} n=5, scAAV9.miP278KY-treated GARS^{(P278KY/+)} n=7. Significance in (FIG. 8A-D, H) was determined by two-way ANOVA with Tukey's HSD posthoc comparisons. Significant changes within axon diameter (E) were determined with a Kolmogorov-Smimov test. *=p<0.05,**=p<0.01, **=p<0.001, **=p<0.0001 represents posthoc significance between miLacZ-treated and scAAV9.miP278KY-treated GARS^{P278KY/+} mice. Values are mean±S.D. All scale bars=100 μm.

FIG. 9A-F shows reduction in mutant GARS expression also alleviates neuropathy in post-disease onset GARS^{(P278KY/+)} mice. (FIG. 9A-B) mi-P278KY treatment at 5 weeks (early onset) or at 9 weeks (late post onset) yields significant increases in grip strength as determined by the wire hang test starting at 5 weeks post treatment in both early- (FIG. 9A) and late-symptomatic (FIG. 9B) GARS^{(P278KY/+)} mice. Treated P278KY mice also gain weight starting 3 weeks post treatment with early-symptomatic mice (A) and 1 week post treatment with late-symptomatic mice (FIG. 9B). When evaluated 7 weeks after treatment, an increase in MW:BW within scAAV9.mi.P278KY early-symptomatic GARS^{(P278KY/+)} mice was observed (FIG. 9C), although scAAV9.miP278KY treatment was unable to improve NCV within this cohort or MW:BW nor NCVs in the late-symptomatic P278KY mice (FIG. 9C-D). However, scAAV9.miP278KY treatment significantly prevented or reversed NMJ breakdown in both early- (FIG. 9E) and late- (FIG. 9E) symptomatic GARS^{(P278KY/+)} mice. All statistics were completed with a One-Way ANOVA with Tukey Posthoc comparisons. Star represents significance between MiLacZ and MiP278KY-treated GARS^{P278KY/+} mice (*=<0.05 **=<0.005 **=<0.001). A=significant difference in fully innervated NMJs, B=significant difference in partially innervated NMJs, and C=significant difference in denervated NMJs. Late-symptomatic Cohort: MiLacZ-treated GARS^{(+/+)} n=6, scAAV9.miP278KY-treated GARS^{(+/+)} n=5-6, mi.LacZ-treated GARS^{(P278KY/+)} n=6, and scAAV9.miP278YK-treated GARS^{(P278KY/+)} n=7. Early-symptomatic Cohort: miLacZ-treated GARS^{(+/+)} n=3, scAAV9.miP278KY-treated GARS^{(+/+)} n=3-4, mi.LacZ-treated GARS^{(P278KY/+)} n=6, and scAAV9.miP278KY-treated GARS^{(P278KY/+)} n=7. Values are mean±S.D. All scale bars=100 μm.

FIG. 10A-C shows escalating reductions of mutant GARS expression within dorsal root ganglia when scAAV9.miP278KY was delivered by ICV compared to IV. (FIG. 10C) In contrast, these gains in peripheral nerve function did not correlate with reductions in mutant GARS expression within liver. N=5 for all experimental groups. IV=intravenous delivery of 1×10¹¹ vg/mouse, ICV=intracerebroventricular delivery, LD=low dose of 8.75×10⁹ vg/mouse, MD=median dose of 5.00×10¹⁰ vg/mouse, and HD=1×10¹¹ vg/mouse. (FIG. 10A) Data were analyzed by a One-Way ANOVA followed by Tukey's posthoc comparisons. a=significant from untreated, wildtype control; b=significant from untreated P278KY control. (FIG. 10B-C) These data were analyzed with a Two-Way ANOVA with Tukey Posthoc comparisons. Star represents significance between wikitype and mutant GARS expression per experimental group (*=<0.05=<0.005 **=<0.001). Values are mean±S.D.

FIG. 11A-D shows long term therapeutic effects of neonatal scAAV9.mi.P278KY treatment. To assess long term effects of scAAV9.mi.P278KY treatment, both bodyweight and grip strength, as determined by the wire hang test, were recorded for 1 year from both GARS^{(P278KY/+)} mice and littermate controls every 6 weeks after being injected with scAAV9.mi.LacZ or scAAV9.mi.P278KY at P0. Remarkably, scAAV9.miP278KY-treated P278KY displayed significant increases in body weight (FIG. 11A) starting at 24 weeks post treatment and grip strength (FIG. 11B) throughout the course of 1 year compared to vehicle control-treated P278KY mice. When evaluated for primary signs of neuropathy at 1 year post-treatment treated-P278KY mice exhibited significantly greater MW:BW ratios (FIG. 11C) and faster NCVs (FIG. 11D). Significance in (FIG. 11A-B) was determined by one-way ANOVA with Tukey's HSD posthoc comparisons. Significance in (FIG. 11C-D) was determined by two-way ANOVA with Tukey's HSD posthoc comparisons. *=p<0.05, **=p<0.01, **=p<0.001, ****=p<0.0001 represents posthoc significance between miLacZ-treated and scAAV9.miP278KY-treated GARS^{P278KY/+} mice for all analyses. MiLacZ-treated GARS^{(+/+)} n=3, scAAV9.miP278KY-treated GARS^{(+/+)} n=3, mi.LacZ-treated GARS^{(P278KY/+)} n=5, and scAAV9.miP278YK-treated GARS^{(P278KY/+)} n=7. Values are mean±S.D.

FIG. 12A-H shows improvements in phenotype negatively correlates with reductions in mutant GARS expression in dorsal root ganglia. (FIG. 12A-B) Scatter plot illustrating the negative correlation between the percentage of the ΔETAQ mutant expression within dorsal root ganglia and nerve conduction velocities quantified from scAAV9.mi.ΔETAQ-treated GARS^{(ΔETAQ/huEx8)} mice within late- (FIG. 12A) and early-(FIG. 12B) symptomatic cohorts. (FIG. 12C-D) Scatter plot illustrating the relationship between the percentage of the ΔETAQ mutant expression within liver and nerve conduction velocities quantified from scAAV9.mi.ΔETAQ-treated GARS^{(ΔETAQ/huEx8)} mice within late-(FIG. 12C) and early- (FIG. 12D) symptomatic cohorts. (FIG. 12E-F) Scatter plot illustrating the negative correlation between the percentage of the P278KY GARS mutant expression within dorsal root ganglia and nerve conduction velocities quantified from scAAV9.mi.P278KY-treated GARS^{(ΔETAQ/huEx8)} mice within late- (FIG. 12E) and early- (FIG. 12F) symptomatic cohorts. (FIG. 12G-H) Scatter plot displaying the association between the percentage of the P278KY GARS mutant expression within liver and nerve conduction velocities quantified from scAAV9.mi.P278KY-treated GARS^{(ΔETAQ/huEx8)} mice within late-(FIG. 12E) and early- (FIG. 12F) symptomatic cohorts.

FIG. 13A-B schematically shows miRNAs (red blocks in panel FIG. 13A) designed to target common regions between the mouse and human GARS cDNAs, while avoiding any sequences containing known GARS mutations associated with CMT2D.

FIG. 14 shows the sequences of GARS miRNAs, each targeting both wild-type and mutant GARS genes.

FIG. 15 shows the sequences of exemplary RNAi-resistant replacement GARS genes.

FIG. 16A-E shows the results of experiments in which neonatal mice were treated systemically with scAAV9-RNAi targeting wild-type GARS (miWT). (FIG. 16A) Total GARS expression was reduced to ˜70% in tissues transduced by AAV9 (dorsal root ganglia and liver) (FIG. 16B). (FIG. 16C-D) 12-week-old mice treated with miWT did not display signs of neuropathy or overt adverse effects. *=p<0.01. Cross sections of the motor branch of the femoral nerve from control mice or mice treated with miWT Gars at twelve weeks of age are shown (FIG. 16E). There were no overt signs of axon loss, demyelination, or axon atrophy.

FIG. 17 is an alignment of the human and mouse GARS gene sequences which shows the portion of the sequences which each GARS miRNAs is designed to target.

### DETAILED DESCRIPTION

The products and methods described herein are used in the treatment of diseases or conditions associated with a mutant glycyl tRNA-synthetase (GARS) gene. In some aspects, the disclosure shows the efficacy of allele-specific RNAi as a potential therapeutic for treating mutations associated with GARS, including Charcot-Marie-Tooth type 2D (CMT2D), caused by dominant mutations in GARS. A de novo mutation in GARS was identified in a patient with a severe peripheral neuropathy, and a mouse model precisely recreating the mutation was produced. These mice developed a neuropathy by 3-4 weeks-of age, validating the pathogenicity of the mutation. RNAi sequences targeting mutant GARS mRNA, but not wild-type GARS, were optimized and then packaged into a viral vector for in vivo delivery demonstrating efficacy in preventing neuropathy in a subject treated at birth and improvement in subjects treated after disease onset.

GARS is one of the aminoacyl-tRNA synthetases that charge tRNAs with their cognate amino acids. Additional information regarding the GARS gene is found at, for example, HGNC(4162), Entrez Gene(2617), Ensembl(ENSG00000106105), OMIM(600287), or UniProtKB(P41250). The encoded enzyme is an (alpha)2 dimer which belongs to the class II family of tRNA synthetases. GARS has been shown to be a target of autoantibodies in the human autoimmune diseases, polymyositis or dermatomyositis. Diseases associated with GARS include, but are not limited to, CMT2D and Distal Hereditary Motor Neuropathy.

In some aspects, the nucleic acid encoding human GARS is set forth in the nucleotide sequence set forth in SEQ ID NO: 69. In various aspects, the products and methods of the disclosure also target isoforms and variants of the nucleotide sequence set forth in SEQ ID NO: 69. In some aspects, the variants comprise 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 69.

In some aspects, the nucleic acid encoding mouse GARS is set forth in the nucleotide sequence set forth in SEQ ID NO: 70. In various aspects, the products and methods of the disclosure also target isoforms and variants of the nucleotide sequence set forth in SEQ ID NO: 70. In some aspects, the variants comprise 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 70.

The disclosure includes the use of RNA interference to inhibit or interfere with the expression mutant GARS to ameliorate and/or treat subjects with diseases or disorders resulting from the mutated GARS gene and the resultant altered version of mRNA. RNA interference (RNAi) is a mechanism of gene regulation in eukaryotic cells that has been considered for the treatment of various diseases. RNAi refers to post-transcriptional control of gene expression mediated by inhibitory RNAs.

As an understanding of natural RNAi pathways has developed, researchers have designed artificial shRNAs and snRNAs for use in regulating expression of target genes for treating disease. Several classes of small RNAs are known to trigger RNAi processes in mammalian cells, including short (or small) interfering RNA (siRNA), and short (or small) hairpin RNA (shRNA) and microRNA (miRNA), which constitute a similar class of vector-expressed triggers [Davidson et al., Nat. Rev. Genet. 12:329-40, 2011; Harper, Arch. Neurol. 66:933-8, 2009]. shRNA and miRNA are expressed in vivo from plasmid- or virus-based vectors and may thus achieve long term gene silencing with a single administration, for as long as the vector is present within target cell nuclei and the driving promoter is active (Davidson et al., Methods Enzymol. 392:145-73, 2005). Importantly, this vector-expressed approach leverages the decades-long advancements already made in the muscle gene therapy field, but instead of expressing protein coding genes, the vector cargo in RNAi therapy strategies are artificial shRNA or miRNA cassettes targeting disease genes-of-interest.

In some embodiments, the products and methods of the disclosure comprise short hairpin RNA or small hairpin RNA (shRNA) to affect GARS expression (e.g., knockdown or inhibit expression). A short hairpin RNA (shRNA/Hairpin Vector) is an artificial RNA molecule with a tight hairpin turn that can be used to silence target gene expression via RNA interference (RNAi). shRNA is an advantageous mediator of RNAi in that it has a relatively low rate of degradation and turnover, but it requires use of an expression vector. Once the vector has transduced the host genome, the shRNA is then transcribed in the nucleus by polymerase II or polymerase Ill, de-pending on the promoter choice. The product mimics pri-microRNA (pri-miRNA) and is processed by Drosha. The resulting pre-shRNA is exported from the nucleus by Exportin 5. This product is then processed by Dicer and loaded into the RNA-induced silencing complex (RISC). The sense (passenger) strand is degraded. The antisense (guide) strand directs RISC to mRNA that has a complementary sequence. In the case of perfect complementarity, RISC cleaves the mRNA. In the case of imperfect complementarity, RISC represses translation of the mRNA. In both of these cases, the shRNA leads to target gene silencing. In some aspects, the disclosure includes the production and administration of a viral vector expressing GARS antisense sequences via miRNA or shRNA. The expression of shRNAs is regulated by the use of various promoters. The promoter choice is essential to achieve robust shRNA expression. In various aspects, polymerase II promoters, such as U6 and H1, and polymerase Ill promoters are used. In some aspects, U6 shRNAs are used.

In some aspects, the disclosure uses U6 shRNA molecules to inhibit, knockdown, or interfere with gene expression. Traditional small/short hairpin RNA (shRNA) sequences are usually transcribed inside the cell nucleus from a vector containing a Pol III promoter such as U6. The endogenous U6 promoter normally controls expression of the U6 RNA, a small nuclear RNA (snRNA) involved in splicing, and has been well-characterized [Kunkel et al., Nature. 322(6074):73-7 (1986); Kunkel et al., Genes Dev. 2(2):196-204 (1988); Paule et al., Nucleic Ac-ids Res. 28(6):1283-98 (2000)]. In some aspects, the U6 promoter is used to control vector-based expression of shRNA molecules in mammalian cells [Paddison et al., Proc. Natl. Acad. Sci. USA 99(3):1443-8 (2002); Paul et al., Nat. Biotechnol. 20(5):505-8 (2002)] because (1) the promoter is recognized by RNA polymerase Ill (poly Ill) and controls high-level, constitutive expression of shRNA; and (2) the promoter is active in most mammalian cell types. In some aspects, the promoter is a type III Pol III promoter in that all elements required to control expression of the shRNA are located upstream of the transcription start site (Paule et al., Nucleic Acids Res. 28(6):1283-98 (2000)). The disclosure includes both murine and human U6 promoters. The shRNA containing the sense and antisense sequences from a target gene connected by a loop is transported from the nucleus into the cytoplasm where Dicer processes it into small/short interfering RNAs (siRNAs).

The disclosure includes sequences encoding inhibitory RNAs to prevent and inhibit the expression of the GARS gene. The inhibitory RNAs comprise antisense sequences, which inhibit the expression of the GARS gene. The disclosure provides nucleic acids encoding GARS miRNAs and guide strands, and RNAi-resistant GARS genes. The disclosure provides a nucleic acid encoding a GARS miRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-25. The disclosure provides a nucleic acid encoding a GARS guide strand comprising at least about 70%, 75, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 26-50. The disclosure provides a nucleic acid comprising an RNAi-resistant GARS gene comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 51-57. The disclosure provides a nucleic acid encoding a GARS miRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-25 and a nucleic acid comprising an RNAi-resistant GARS gene comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 51-57.

Exemplary GARS miRNAs comprise a full length miRNA antisense guide strand comprising the polynucleotide sequence set out in any one or more of SEQ ID NOs: 1-25, or a variant thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs 1-25. Corresponding final processed guide strand sequences are respectively set out in the polynucleotide sequence set out in any one or more of SEQ ID NOs: 26-50, or a variant thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs 26-50. Exemplary RNAi-resistant replacement GARS genes are set out in any one of more of SEQ ID NOs: 51-57, or a variant thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs: 51-57.

In some aspects, one or more copies of these sequences are combined into a single vector. Thus, the disclosure includes vectors comprising a nucleic acid of the disclosure or a combination of nucleic acids of the disclosure. Embodiments of the disclosure utilize vectors (for example, viral vectors, such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle, or small molecule) to deliver the nucleic acids disclosed herein. In some aspects, the viral vector is an AAV. In some aspects, the AAV lacks rep and cap genes. In some aspects, the AAV is a recombinant AAV (rAAV) or a self-complementary recombinant AAV (scAAV). In some aspects, the AAV has a capsid serotype selected from the group consisting of: AAV-1, AAV-2, AAV-3, AAV-4, AAV-5, AAV-6, AAV-7, AAV-8, AAV-9, AAV-10, AAV-11, AAV-12, AAV-13, AAV-anc80, AAV rh.74, AAV rh.8, and AAVrh.10.

In some embodiments, the viral vector is an AAV, such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), AAVrh.10 (i.e., an AAV containing AAVrh.10 ITRs and AAVrh.10 capsid proteins), AAV11 (i.e., an AAV containing AAV11 ITRs and AAV11 capsid proteins), AAV12 (i.e., an AAV containing AAV12 ITRs and AAV12 capsid proteins), or AAV13 (i.e., an AAV containing AAV13 ITRs and AAV13 capsid proteins).

DNA plasmids of the disclosure comprise rAAV genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, and AAV rh.74. In some aspects, AAV DNA in the rAAV genomes is from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, and AAV rh.74. Other types of rAAV variants, for example rAAV with capsid mutations, are also included in the disclosure. See, for example, Marsic et al., Molecular Therapy 22(11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

In some embodiments, the viral vector is a pseudotyped AAV, containing ITRs from one AAV serotype and capsid proteins from a different AAV serotype. In some embodiments, the pseudo-typed AAV is AAV2/9 (i.e., an AAV containing AAV2 ITRs and AAV9 capsid proteins). In some embodiments, the pseudotyped AAV is AAV2/8 (i.e., an AAV containing AAV2 ITRs and AAV8 capsid proteins). In some embodiments, the pseudotyped AAV is AAV2/1 (i.e., an AAV containing AAV2 ITRs and AAV1 capsid proteins).

In some embodiments, the AAV contains a recombinant capsid protein, such as a capsid protein containing a chimera of one or more of capsid proteins from AAV1, AAV2, AAV3, AAV4, AAV5, AAV6, AAV7, AAV8, AAV9, AAVrh74, AAVrh.8, or AAVrh.10, AAV10, AAV11, AAV12, or AAV13. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art.

In some embodiments, the disclosure utilizes AAV to deliver inhibitory RNAs which target the GARS mRNA to inhibit mutant GARS expression. AAV is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 {1983); the complete genome of AAV-3 is provided in GenBank Accession No. NC_1829; the complete genome of AAV-4 is provided in GenBank Accession No. NC_001829; the AAV-5 genome is pro-vided in GenBank Accession No. AF085716; the complete genome of AAV-6 is provided in GenBank Accession No. NC_00 1862; at least portions of AAV-7 and AAV-8 genomes are pro-vided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cis-acting sequences directing viral DNA replication (rep), encapsidation/packaging and host cell chromo-some integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hearty virus. It easily withstands the conditions used to inactivate adenovirus, making cold preservation of AAV less critical. AAV may be lyophilized and AAV-infected cells are not resistant to superinfection. In some aspects, AAV is used to deliver shRNA under the control of a U6 promoter. In some aspects, AAV is used to deliver snRNA under the control of a U7 promoter. In some aspects, AAV is used to deliver an RNAi-resistant replacement GARS gene under the control of a chicken β-actin promoter.

In some embodiments, the AAV lacks rep and cap genes. In some embodiments, the AAV is a recombinant linear AAV (rAAV), a single-stranded AAV, or a recombinant self-complementary AAV (scAAV).

Recombinant AAV genomes of the disclosure comprise one or more AAV ITRs flanking a polynucleotide encoding, for example, one or more GARS inhibitory RNAs or GARS miRNAs. The genomes of the rAAV provided herein either further comprise an RNAi-resistant replacement GARS gene, or the RNAi-resistant replacement GARS gene is present in a separate rAAV. The miRNA- and replacement GARS-encoding polynucleotides are operatively linked to transcriptional control DNAs, for example promoter DNAs, which are functional in a target cell. Commercial providers such as Ambion Inc. (Austin, Tex.), Darmacon Inc. (Lafayette, Colo.), InvivoGen (San Diego, Calif.), and Molecular Research Laboratories, LLC (Hemdon, Va.) generate custom inhibitory RNA molecules. In addition, commercial kits are available to produce custom siRNA molecules, such as SILENCER™ siRNA Construction Kit (Ambion Inc., Austin, Tex.) or psiRNA System (InvivoGen, San Diego, Calif.).

DNA plasmids provided comprise rAAV genomes described herein. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-B1 and AAV rh74. Production of pseudotyped rAAV is disclosed in, for example, WO 01/83692 which is incorporated by reference herein in its entirety. Exemplary rAAV comprising AAV-9 capsid proteins and AAV-2 ITRs are provided.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbiol. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mo1. Cell. Biol. 5:3251 (1985); McLaughlin et al., J. Virol., 62:1963 (1988); and Lebkowski et al., 1988 Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine, 13:1244-1250 (1995); Paul et al., Human Gene Therapy, 4:609-615 (1993); Clark et al., Gene Therapy, 3:1124-1132 (1996); U.S. Pat. Nos. 5,786,211; 5,871,982; 6,258,595; and McCarty, Mol. Ther., 16(10): 1648-1656 (2008). The foregoing documents are hereby incorporated by reference in their entirety herein, with particular emphasis on those sections of the documents relating to rAAV production. The production and use of self-complementary (sc) rAAV are specifically contemplated and exemplified.

Further provided are packaging cells that produce infectious rAAV. Packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) are thus provided herein. The genomes of the rAAV lack AAV rep and cap DNA, that is, there is no AAV rep or cap DNA between the ITRs of the genomes of the rAAV.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

Compositions comprising the nucleic acids and viral vectors of the disclosure are provided. Compositions comprising delivery vehicles (such as rAAV) described herein are provided. In various aspects, such compositions also comprise a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁶, or more DNase resistant particles (DRP) [or viral genomes (vg)] per ml.

Methods of transducing a target cell with a delivery vehicle (such as rAAV), in vivo or in vitro, are contemplated. The in vivo methods comprise the step of administering an effective dose, or effective multiple doses, of a composition comprising a delivery vehicle (such as rAAV) to an animal (including a human patient) in need thereof. If the dose is administered prior to development of a disorder/disease, the administration is prophylactic. If the dose is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the invention is CMT2D. In families known to carry pathological GARS mutations, the methods can be carried out in a before the onset of disease. In other patients, the methods are carried out after diagnosis.

Molecular, biochemical, histological, and functional outcome measures demonstrate the therapeutic efficacy of the methods. Outcome measures are described, for example, in Chapters 32, 35 and 43 of Dyck and Thomas, Peripheral Neuropathy, Elsevier Saunders, Philadelphia, Pa., 4^{th} Edition, Volume 1 (2005) and in Burgess et al., Methods Mol. Biol., 602: 347-393 (2010). Outcome measures include, but are not limited to, one or more of the reduction or elimination of mutant GARS mRNA or protein in affected tissues, GARS gene knockdown, increased body weight and improved muscle strength. Others include, but are not limited to, nerve histology (axon number, axon size and myelination), neuromuscular junction analysis, and muscle weights and/or muscle histology. Others include, but are not limited to, nerve conduction velocity-ncv, electromyography-emg, and synaptic physiology.

In the methods of the disclosure, expression of the mutant GARS allele is inhibited by at least 10, at least 20, at least 30, at least 40, at least 50, at least 60, at least 70, at least 80, at least 90, at least 95, at least 98 percent, at least 99 percent, or 100 percent. In the methods, expression of the wild-type GARS allele is inhibited by at least 10, at least 20, at least 30, at least 40, at least 50, at least 60, at least 70, at least 80, at least 90, at least 95, at least 98 percent, at least 99 percent, or 100 percent.

Combination therapies are also contemplated by the invention. Combination as used herein includes both simultaneous treatment and sequential treatments. Combinations of methods described herein with standard medical treatments and supportive care are specifically contemplated, as are combinations with therapies such as HDAC6 inhibition [Benoy et al., Brain, 141(3):673-687 (2018)].

Administration of an effective dose of a nucleic acid, viral vector, or composition of the disclosure may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration and serotype(s) of AAV components of the rAAV (in particular, the AAV ITRs and capsid protein) of the invention may be chosen and/or matched by those skilled in the art taking into account the infection and/or disease state being treated and the target cells/tissue(s) that are to express the miRNAs.

In particular, actual administration of delivery vehicle (such as rAAV) may be accomplished by using any physical method that will transport the delivery vehicle (such as rAAV) into a target cell of an animal. Administration includes, but is not limited to, injection into muscle, the bloodstream and/or directly into the nervous system or liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as neurons. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction of cells with rAAV of the invention results in sustained expression of GARS miRNAs and RNAi-resistant replacement GARS gene. The present invention thus provides methods of administering/delivering rAAV which express GARS miRNAs and an RNAi-resistant replacement GARS gene to an animal, preferably a human being. These methods include transducing cells and tissues (including, but not limited to, peripheral motor neurons, sensory motor neurons, tissues such as muscle, and organs such as liver and brain) with one or more rAAV described herein. Transduction may be carried out with gene cassettes comprising cell-specific control elements.

The term “transduction” is used to refer to, as an example, the administration/delivery of GARS miRNAs and RNAi-resistant replacement GARS genes to a target cell either in vivo or in vitro, via a replication-deficient rAAV described herein resulting in the expression of GARS miRNA and the RNAi-resistant replacement GARS gene by the target cell.

Thus, methods are provided of administering an effective dose (or doses, administered essentially simultaneously or doses given at intervals) of rAAV described herein to animal in need thereof.

### EXAMPLES

Aspects and embodiments of the invention are illustrated by the following examples. Example 1 describes the clinical evaluation and mutation analysis of a CMT2D patient. Example 2 describes GARS expression studies. Example 3 describes CMT2D mouse models. Example 4 describes miRNAs specific for the GARS gene. Example 5 describes the production of scAAV9.mi.ΔETAQ. Example 6 describes the neonatal delivery of scAAV9.mi.P278KY and scAAV9.mi.ΔETAQ to mice. Example 7 describes the delivery of gene therapy constructs to post-onset mice. Example 8 describes rAAV9-miGARS/rGARS vector and use. Example 9 describes experiments relating to the level of GARS expression that results in a normal phenotype. Example 10 shows that ΔETAQ GARS affects the primary function of the enzyme. Example 11 shows that Example 11 shows that ΔETAQ GARS showed slightly aberrant interaction with NRP1.

### Example 1

### Clinical Evaluation and Mutation Analysis of a CMT2D Patient

A patient-specific GARS mutation was chosen to exemplify the methods and products provided herein. The GARS mutation was identified in a now six-year-old female who presented clinically by displaying decreases in muscle tone, head lag, axillary slippage, mild tongue atrophy, ligamentous laxity in the hands and feet, and excessive retraction of the chest wall starting at 13 months of age. Muscle biopsy at 15 months indicated neurogenic changes consistent with neuropathy. This included marked atrophy of type I and II fibers, and no evidence of myofiber necrosis, degeneration, or regeneration; nor of dystrophic or inflammatory myopathy. EMG and nerve conduction studies were also consistent with motor neuron disease. After negative tests for spinal muscular atrophy, whole-exome sequencing analysis revealed that the patient is heterozygous for an in-frame, 12 nucleotide deletion in exon 8 of the glycyl-tRNA synthetase (GARS) gene (c.894_905del; NM_002047.2).

More specifically, the proband was clinically evaluated at Texas Children's Hospital (Houston, Tex.) under Institutional Review Board approved protocols. Clinical data were obtained after written informed consent from the proband's parents. Diagnostic, whole-exome sequencing (XomeDxPlus) was performed by GeneDx (Gaithersburg, Md.). For allele-specific Sanger sequencing, we first isolated DNA from patient-derived primary fibroblasts. Cells were treated with trypsin according to the Wizard Genomic DNA Purification Kit (Promega) protocol. PCR amplification was performed to obtain a 381 bp region including GARS exon 8 using PCR SuperMix (ThermoFisher Scientific). PCR products were cleaned according to the QiaQuick PCR Purification Kit protocol and cloned into the pCR4-TOPO vector using the TOPO TA Cloning Kit (ThermoFisher Scientific). Vectors were then transformed into One Shot TOP10 Chemically Competent E. coli cells (ThermoFisher Scientific) and plated on ampicillin-containing LB agar plates. Plasmid DNA from six isolated colonies was purified and Sanger sequenced using the PCR primers. Five colonies contained plasmids with the amplicon of the wild-type allele and one colony contained plasmids with the amplicon of the mutant allele. Primers used for the PCR reaction: forward 5′GCATTGCCAAAGTAGTACTGC 3′ (SEQ ID NO: 58); and reverse 5′ CCTGACTCTGATCAGTCCAGATCG 3′ (SEQ ID NO: 59).

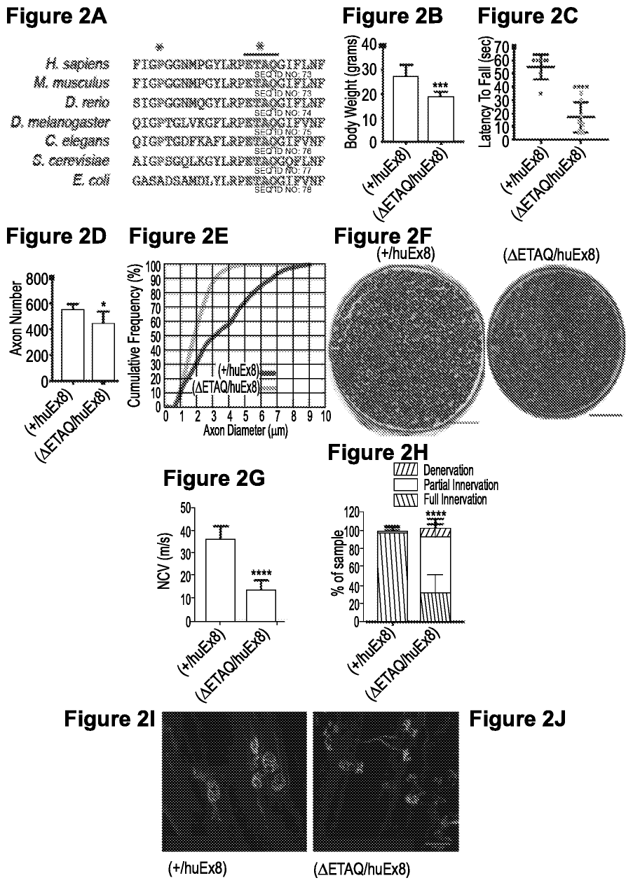

This mutation resulted in the deletion of four amino acids in the GARS protein (p.Glu299_Gln302del; NP_002038.2) hereafter, referred to as ΔETAQ. No other potentially pathogenic mutation was identified at another locus that could potentially explain the severity of the neuropathy by a dual molecular diagnosis. Neither parent carries the identified GARS mutation, nor does the patient's twin brother, indicating a de novo mutation. GARS functions as a dimer to ligate glycine onto cognate tRNA molecules. The substrate glycine is bound within a pocket of each monomer, and one tRNA molecule associates with each half of the dimer. Importantly, the ΔETAQ GARS mutation results in the deletion of four amino-acid residues that are conserved from human to bacteria and that reside within the glycine-binding pocket (FIG. 2A) [Qin et al., J. Biol. Chem., 289: 20359-20369 (2014)].

### Example 2

### GARS Expression Studies

To determine if the ΔETAQ GARS mutation affects mRNA expression or stability, RNA-seq was performed to assess the expression of wild-type and mutant alleles in patient primary dermal fibroblasts.

For RNA expression studies, RNA was isolated from patient fibroblasts using the RNeasy Mini Kit (Qiagen) per the manufacturer's protocol. cDNA samples were generated from 1 μg of RNA using the High-Capacity cDNA reverse transcription kit (Applied Biosystems) following the manufacturer's instructions. The resulting cDNA was used to amplify a 224 base-pair product flanking the region bearing the ΔETAQ GARS mutation. The reaction was column purified and the product was analyzed for quality via gel electrophoresis. To prepare the sample for next-generation sequencing, the product was digested and “tagmented” using Tn5 transposase. The library was amplified by PCR using Kapa Hifi DNA polymerase and Illumina-compatible indexing primers. Final library fragment size and purity was determined via gel electrophoresis, and fragments were column purified and sequenced on the Illumina MiSeq with paired 155-bp reads. All primer sequences are available upon request. Overlapping reads were merged using PEAR (v0.9.6) and aligned using bwa mem (v0.7.12) to custom references containing the wild-type exon-7:exon-8 junction or the ΔETAQ-containing equivalent. A custom python script (available upon request) was used to count reads with higher-scoring alignment to each junction. Uninformative reads (e.g., those not spanning the mutation) were disregarded.

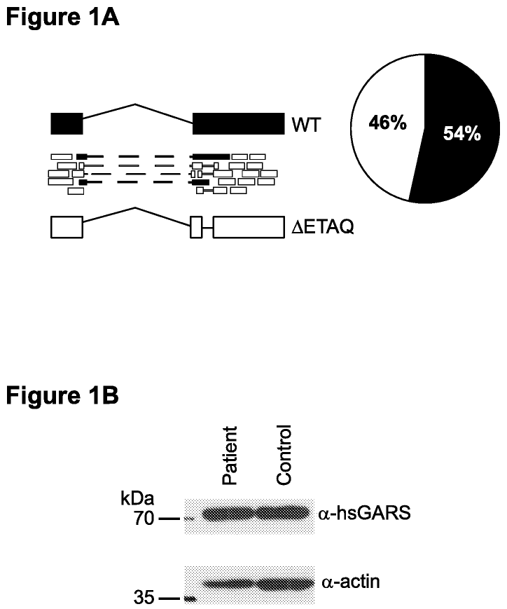

These analyses revealed an even distribution of wild-type (53.7%) and ΔETAQ (46.3%) RNA-seq reads indicating that ΔETAQ GARS does not dramatically affect transcript levels (FIG. 1A).

To determine if ΔETAQ GARS impacts GARS protein levels, we performed a Western blot analysis on whole-cell lysates from patient cells compared to a control primary dermal fibroblast cell line (i.e., bearing no GARS mutations).

For protein expression studies, cells were cultured and harvested under normal conditions. Proteins were isolated in 1 mL cell lysis buffer [990 μL RIPA Lysis Buffer (ThermoFisher Scientific)+10 μL 100× Halt Protease Inhibitor (ThermoFisher Scientific)]. Protein concentrations were quantified using the Thermo Scientific Pierce™ BCA Protein Assay Kit (ThermoFisher Scientific) and 10 μg of protein per sample was analyzed via western blot. Each protein sample was prepared in 1×SDS-sample buffer (ThermoFisher Scientific) plus 5 μL 2-me beta-mercaptoethanol (β-ME) and boiled at 99° C. for 10 minutes. Samples were electrophoresed on pre-cast 4-20% tris-glycine gels (ThermoFisher Scientific) at 150V for 1 hour. Proteins were transferred onto a polyvinylidene difluoride (PVDF) membrane at 25V for 1.5 hours. The membrane was incubated for 1 hour at room temperature with the respective primary antibody at the following dilutions in blocking solution: anti-GARS 1:1,000; anti-neuropilin-1 (Abcam) 1:1,000; and anti-actin (Sigma Aldrich) 1:5,000. Membranes were then rinsed 3× in 1×TBST to remove unbound antibody and incubated with the respective HRP-conjugated secondary antibody at 1:10,000. Membranes were rinsed in 1×TBST and exposed using SuperSignal West Dura substrate and enhancer (ThermoFisher Scientific).

These experiments did not reveal an observable difference in total GARS protein levels in the affected fibroblasts compared to the control cell line, consistent with the mutant protein being expressed and stable (FIG. 1B).

### Example 3

### Mouse Models

Three mouse models of CMT2D harboring dominant mutations in GARS, and causing peripheral neuropathy are provided herein. Two of these carry mouse-specific alleles and have been previously described in Sebum et al., Neuron, 51: 715-726 (2006) and Achilli et al., Disease Models & Mechanisms, 2: 359-373 (2009), the third carries the human disease-associated mutation described Example 1, and its creation is described below. Together, the models provide a range of severity and allow multiple alleles, including a human allele, to be used in preclinical testing. All the models have excellent face validity, with length-dependent peripheral neuropathy, and construct validity, with dominant mutations in mouse Gars gene underlying their phenotype.

The two mouse-specific alleles were identified based on their neuromuscular phenotype. The P278KY allele (a.k.a. Nmf249) was found at Jackson Laboratories, and causes a severe neuropathy with ˜25% loss of myelinated peripheral axons, reduced axon diameter, reduced nerve conduction velocity, reduced grip strength, muscle atrophy, and denervation, partial innervation, and transmission defects at the neuromuscular junction (NMJ). The milder C201R allele was found in a chemical mutagenesis program in the UK, and has little or no axon loss, but shows reduced axon diameters, reduced conduction velocity, reduced grip strength, muscle atrophy, and similar, but milder, NMJ defects. Both strains are affected relatively early, with P278KY showing an overt phenotype by 2-3 weeks of age, and C201R by 4-6 weeks of age. Both also have length-dependent motor innervation defects. The severe P278KY allele shows genetic-background-dependent lethality at approximately 8 weeks in an inbred C57BL/6J background. The C201R mice and P278KY mice on a mixed genetic background survive well over one year. These alleles are not found in CMT2D patients, but the C201 and P278 residues are conserved.

As described in Example 1, the human disease-associated variant is a 12-base pair deletion in exon eight of the human GARSgene, removing four amino acids (ΔETAQ or Ex8D12) at positions 245-8 in the protein. (Note, the human protein is numbered from the second ATG, the cytosolic form of GARS, not the first ATG, which produces the mitochondrial isoform. Thus ETAQ 245-8 is 11 amino acids C-terminal to the mouse P278KY allele—P234 in humans.)

To definitively validate the pathogenicity of ΔETAQ GARS in vivo, we engineered the mouse model in which the patient-derived mutation was introduced into exon 8 of the mouse GARS gene (GARS^{ΔETAQ/+}) using CRISPR/Cas9 genome-editing technology.

As a control, the sequence of wild type human GARS exon 8 (huEx8) was also introduced into the mouse genome. For GARS^{huEx8/+}, the mouse exon 8 sequence was replaced with a double-stranded donor vector containing the human exon 8 sequence. The donor vector was synthesized by recombineering a 10 kb sequence containing the mouse exon 8 sequence flanked by a 2.8 kb long 5′ arm of homology and a 7 kb 3′ arm of homology isolated from a C57BL/6J BAC library into a retrieval vector containing short arms of homology for this fragment. The mouse exon 8 sequence was then removed from the vector and replaced with the human exon 8 sequence via restriction digest and subsequent ligation with T4 ligase. As for GARS^{ΔETAQ/+}, the donor construct consisted of a ss-oligonucleotide sequence spanning the first 52 bases of mouse exon 8 with short arms of homology (see below for sequence) containing a 12-base deletion (bases 12-23 of exon 8) referred to as ΔETAQ.

Preparation and microinjection of Crispr/Cas9 reagents was performed as described in (Qin et al., Curr Protoc Mouse Biol. 2016; 6(1):39-66). All components including Cas9 mRNA (100 ng/μl, either TriLink or synthesized by in vitro transcription), sgRNA, guides 144 and 1340 (50 ng/μl; guide sequence below), and each donor vector (20 ng/μl plasmid DNA or 100 ng/ul ssODN) were injected into the male pronucleus and cytoplasm of ˜300 zygotes at the pronuclei stage. All zygotes were isolated from a superovulated FVB/NJ (JAX stock #001600) females mated with C57BL/6NJ (JAX stock #005304) males. After, groups of 15-25 blastocysts were transferred into the uterus of pseudopregnant females.

ssODN donor:
(SEQ ID NO: 60)
AGTTTACTTGTAACAGGCTTTGTTTTATTGGAAGCACATTGTCTTACTT
GTAATAGACTGGTTTATTTAATTTTATAGATACTTGAGACCGGGGATTT
TCtTGAATTTCAAACGACTTTTGGAATTCAAC
sgRNA 144:
(SEQ ID NO: 61)
aaaattccctgtgcagtttc
sgRNA 1340:
(SEQ ID NO: 62)
tcag aaatgag atctcacct

Transgenic mice were genotyped based on the presence of either the humanized exon 8 or ΔETAQ constructs. Genomic DNA was prepared from tail biopsy lysed with proteinase K incubation. Primers HuEx8F0_F:CATAACATCACGCGTGGTTCC (SEQ ID NO: 63) and HuEx8R0_R:CAAGTGTGGCGGTTTCCATC (SEQ ID NO: 64) that span the 2.8 kb 5′ arm of homology to the 3′ end of GARS exon 8 and subsequent Sanger sequencing with HuEx8R0_R was used to identify human single nucleotide polymorphisms in exon 8 of GARS within GARS^{huEx8} founders and subsequent generations. Primers delETAQF0_F: GGCCATAAGCATAATTTTACTGTG (SEQ ID NO: 65) and ΔETAGF0_R:TACAACAGAAACAAACTGTGGTCA (SEQ ID NO: 66) with subsequent Sanger sequencing with ΔETAGF0_R to detect the 12 base-pair deletion in bases 13-24 in GARS^{(ΔETAQ/+)} founders and subsequent generations.

For subsequent preclinical studies, GARS^{ΔETAQ/+} mice were crossed to GARS^{huEx8/huEx8}, a control mouse model was engineered that harbors a “humanized” wild-type GARS exon 8 replacement in the mouse gene. The GARS gene is highly conserved, including intron/exon structure, and the fifty amino acids encoded by exon 8 are 100% identical between mouse and human, although there are some silent single-nucleotide differences between the mouse and human GARS/GARS exon 8 that could affect allele-specificity of gene silencing, thereby necessitating humanizing the wild-type mouse exon.

This breeding produced a cohort of GARS^{ΔETAQ/huEx8} mice with GARS^{huEx8/+} littermate controls. Reverse transcriptase-PCR using cDNA isolated from sciatic nerve of heterozygous mice revealed co-expression of ΔETAQ and wild-type GARS (Figure S3A). Primers GARS2F_CTCCCACCACTGGCAATGAC (SEQ ID NO: 67) and GARS2R_CTCACTCAGCAGCAGCTCC (SEQ ID NO: 68) were used to amplify a portion of the GARS open reading frame spanning GARS exon 8 from first-strand cDNA generated from sciatic nerve RNA isolated from GARS^{(+/huEx8)} and GARS^{(ΔETAQ/huEx8)} mice. Humanized exon 8 and ΔETAQ transcript sequences were identified with Sanger sequencing and primer GARS2F.

Mice were housed in pressurized individually ventilated (PIV) racks in the research animal facility at The Jackson Laboratory and provided food and water ad libitum. All mouse husbandry and experimental procedures were conducted according to the NIH Guide for Care and Use of Laboratory Animals and were reviewed and approved by The Animal Care and Use Committee of The Jackson Laboratory. GARS (CAST; B6-GARS^{Nmf249}/Rwb (referred to as GARS^{P278KY/+}) are previously described in (17). The official strain designations of the newly engineered mouse models are B6; FVB-GARS<em1Rwb>/Rwb (referred to as GARS^{huEx8}) and B6; FVB-GARS<em2Rwb>/Rwb (referred to as GARS^{(ΔETAQ/+)}). Unless otherwise noted, all experimental cohorts used for direct comparisons consisted of littermate animals to match strain and age to the greatest extent possible.

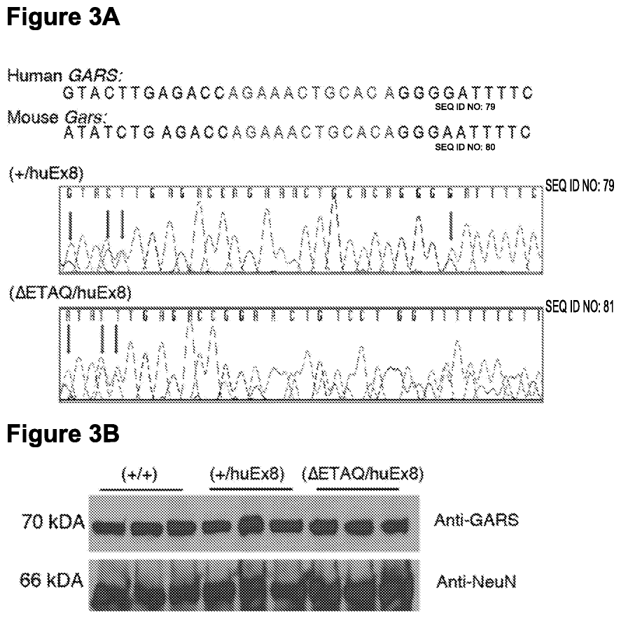

At the protein level, a Western blot analysis of mouse brain homogenates using a polyclonal anti-GARS antibody confirmed that ΔETAQ GARS did not alter GARS protein levels, suggesting that a stable transcript and protein products are produced from the ΔETAQ allele similar to our results with patient fibroblasts (FIG. 3B). Whole brain samples were isolated from animals immediately after they were euthanized by CO₂ inhalation. The tissues were frozen in liquid nitrogen and stored at −80′C. Samples were homogenized using a mortar and pestle followed by a Dounce homogenizer in 1% NP-40 in phosphate buffered saline supplemented with Protease Inhibitor Cocktail Tablets (Roche, Basal, Switzerland) then centrifuged at 14,000 g twice for 5 minutes at 4′C. Cleared homogenates were then sonicated at 4′C and centrifuged again at 14,000 g for 5 minutes. 20 μg of protein was then analyzed by immunoblot. Protein lysates were resolved on Mini-PROTEAN 4-15% Tris-Glycine gels (BioRad, Hercules, Calif.) and transferred to an Invitrolon & Immobilon-P PVDF membrane for western blot analysis. Membranes were blocked with 5% skim milk in TBST (1× Tris-buffered saline, 0.1% Tween-20), and incubated overnight with anti-GARS (rabbit, Abcam, 1:1000 dilution) and anti-NeuN (mouse monoclonal, Cell Signaling, 1:1000) diluted in blocking solution at 4′C. Following three 10 minute washes in TBST, the blots were incubated with the appropriate horseradish peroxidase-conjugated secondary antibodies (PerkinElimer, Boston Mass.) diluted in blocking solution. After three 10 minute washes in TBST, the blots were developed using Western Lightening Plus-ECL, Enhanced Chemiluminescence Substrate (Perkin Elmer, Waltham, Mass.)

At 12 weeks-of-age, GARS^{ΔETAQ/huEx8} and GARS^{huEx8/+} littermates were evaluated for features of primary neuropathy, as observed in other mouse models of CMT2D (Seburn et al., supra; Achilli et al., supra). Grip strength was evaluated by wire hang test [Motley et al., PLoS Genet., 7: e1002399 (2011)] to evaluate gross muscle strength and endurance. Nerve conduction studies, motor nerve histology and analysis, neuromuscular junction immunofluorescence and analysis, and body weight evaluation were completed as previously described in [Motley et al., supra; Morelli et al., Cell. Rep., 18: 3178-3191 (2017)]. Like these previous models, GARS^{ΔETAQ/huEx8} mice displayed overt neuromuscular dysfunction and a significant reduction in body weight (p=0.0006) and grip strength (p=0.0002) compared to huEx8/+ controls (FIG. 2B-C). Histological changes in GARS^{ΔETAQ/huEx8} mice were observed in cross sections of the motor branch of the femoral nerve, including an overall decrease in axon number (p=0.0293) and axon diameter (p=<0.0001) (FIG. 2D-F). Nerve conduction velocities (NCV) were also reduced by 62%, falling from 35±6.29 m/s in control animals to 13.5±4.1 m/s (p=0.0002) in the sciatic nerve in mutant mice (FIG. 2G). This decrease was consistent with NCVs observed in other mouse GARS neuropathy models and in some patients with GARS-mediated peripheral neuropathy (CMT2D). The loss of motor axons resulted in a concomitant disruption of neuromuscular junctions (NMJs) in distal muscles. While postsynaptic receptor fields of NMJs in the plantaris muscle were fully occupied by motor nerve terminals in control littermates, 60%±14.2% of NMJs were partially occupied and 8.5%±9.9% were completely denervated in mice that expressed the ΔETAQ mutation (FIG. 2H-J). Thus, GARS^{ΔETAQ/huEx8} mice display primary features of peripheral neuropathy similar to those observed in established mouse models of CMT2D, confirming that the ΔETAQ GARS mutation is indeed pathogenic and causative of the neuropathy observed in the patient described in Example 1.

### Example 4

### MicroRNAs Specific for the GARS Gene

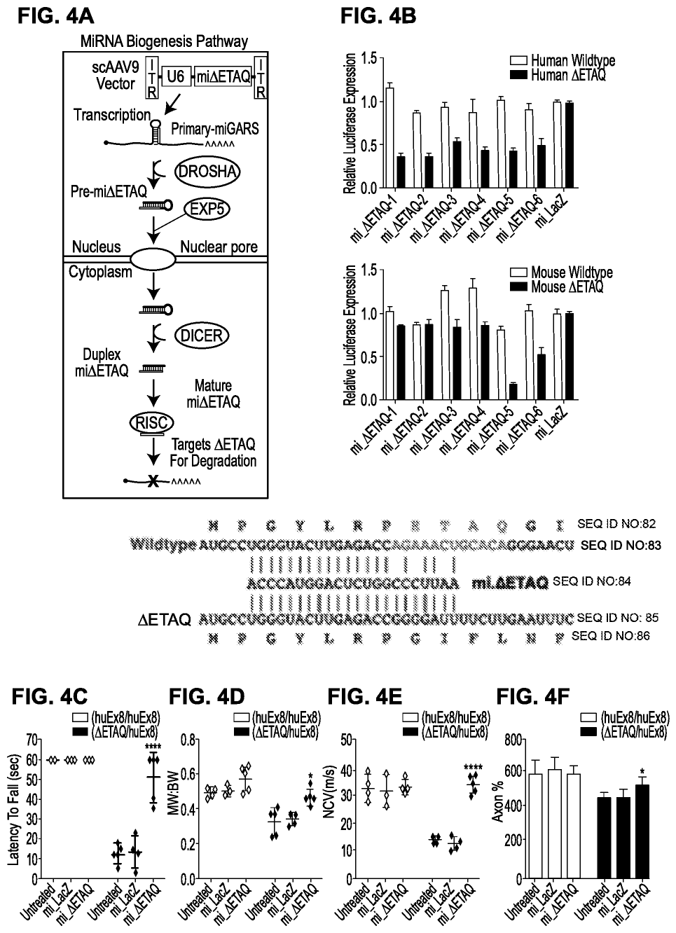

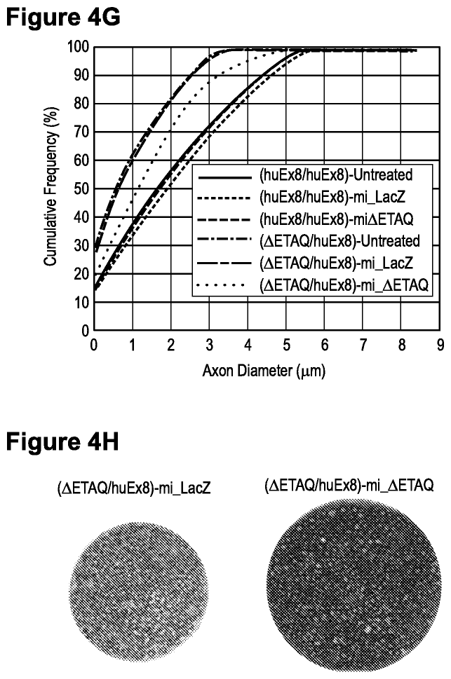

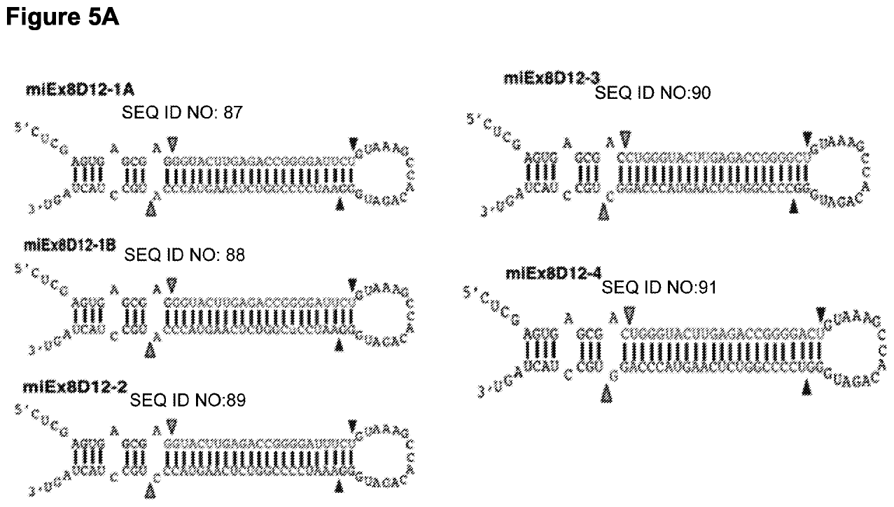

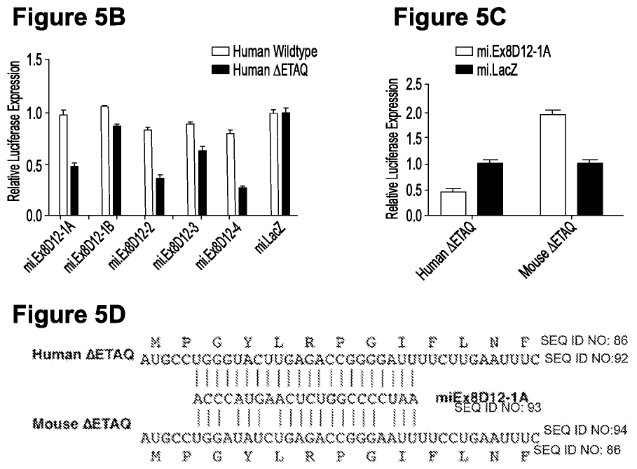

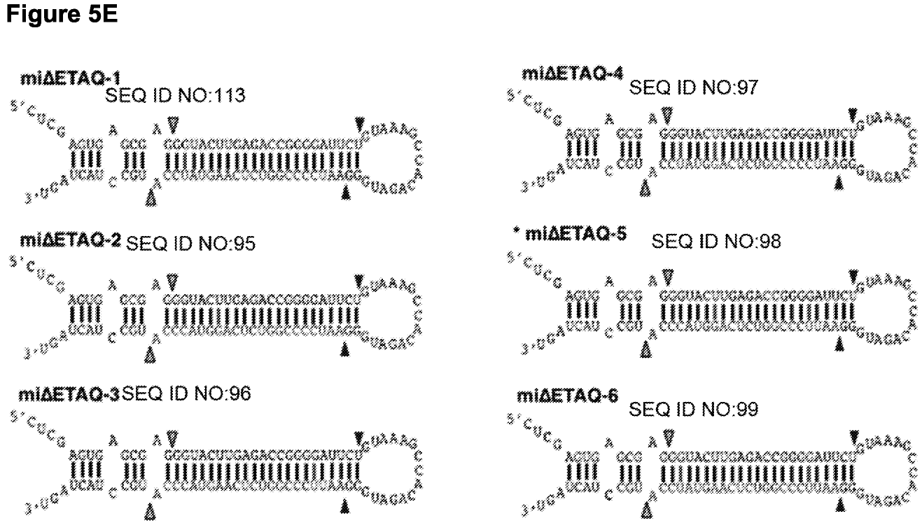

To achieve allele-specific knockdown of mutant GARS using RNAi, we first engineered a miRNA shuttle designed to specifically target ΔETAQ transcripts for degradation in the GARS^{ΔETAQ/huEx8} mice (FIG. 4A-B, FIG. 5). Specifically, we designed six different mir-30 based artificial microRNAs shuttles (mi.ΔETAQ1-6) with a mature guide strand designed to specifically target both human and mouse mutant GARS ΔETAQ mRNA for degradation (FIG. 4B, FIG. 5).

The artificial microRNAs included 22 nt mature miRNA length, perfect antisense complementarity to the target mRNA (GARS; GARS), <60% GC content of the mature duplex, and guide-strand biasing, such that the last 4 nucleotides of the antisense 5′ end were A:U rich, and the last 4 nucleotides of the antisense 3′ end were G:C rich. The mutant GARS-targeting microRNA constructs had seed match regions focused on the differing nucleotides present in the mutant P278KY or ΔETAQ alleles, with intentional mismatches between the mature miRNA guide strand the wild-type GARS/GARS. DNAs encoding the microRNA constructs were ligated to a U6T6 vector (via XhoI and XbaI) overnight. This vector contains a mouse U6 promoter and an RNA polymerase Ill termination signal (6 thymidine nucleotides). The DNAs were cloned into XhoI+XbaI restriction sites located between the 3′ end of the U6 promoter and the termination signal (SpeI on the 3′ end of the DNA template for each miRNA has complementary cohesive ends with the XbaI site). The ligation product was transformed into chemically competent E-coli cells with a 42° C. heat shock and incubated at 37° C. shaking for 1 hour before being plated on kanamycin selection plates. The colonies were allowed to grow overnight at 37°. The following day they were mini-prepped and sequenced for accuracy

The resulting vectors were used in an initial in vitro dual-luciferase screening assay [Boudreau et al., pp. 19-37 in Harper, Ed., RNA Interference Techniques, Human Press, New York, Vol. 1 (2011)], in which the ΔETAQ or wikitype GARS target sequences were cloned into the 3′ UTR of sea pansy (Renilla reniformis) luciferase and used firefly luciferase as a standard. The dual luciferase plasmids were created using the Psicheck2 vector (Promega), with a Firefly luciferase cassette serving as a transfection control, and the various GARS gene target regions cloned downstream of the Renilla luciferase stop codon, thereby serving as a 3′ UTR. HEK293 cells were co-transfected (Lipofectamine-2000, Invitrogen) with the appropriate dual luciferase reporter and an individual U6.miRNA expression plasmid in a 1:5 molar ratio. GARS silencing was determined by measuring Firefly and Renilla activity 24 hours post transfection, using the Dual-Luciferase Reporter Assay System (Promega). Triplicate data were averaged and knockdown significance was analyzed using two-way ANOVA. Results are presented as the mean ratio of Renilla to firefly ±SEM.

Several of these constructs proved effective at specifically silencing the ΔETAQ mutant allele, and miEx8D12-1A was chosen as a lead candidate (FIG. 5B). However, when this luciferase assay was repeated using the mouse ΔETAQ GARS gene as the target, the lead miRNA was unable to knock down the mutant allele (FIG. 5C). This is likely due to slight differences in the mouse mRNA sequence relative to the human, despite the amino acid sequences being identical (FIG. 5D). In order to have a miRNA that could be tested in the mouse model, we created six variants of miEx8D12-1A, named miΔETAQ1-6 (FIG. 5E). These miRNAs had one or two point mutations relative to the original sequence, in order to increase complementarity to the mouse ΔETAQ gene, without losing the ability to target the human ΔETAQ GARS. miΔETAQ-5 was highly effective against both the human and mouse mutant allele, was renamed “mi.ΔETAQ,” and used in all further in vivo studies (FIG. 4).

### Example 5

### Production of scAAV9.mi.ΔETAQ

After in vitro testing was completed, mi.ΔETAQ (FIG. 4B) was cloned into a scAAV9 for in vivo delivery. The scAAV9 named “scAAV9.mi.ΔETAQ” also contained a CMV promoter-driven eGFP reporter gene. The scAAV9.mi.ΔETAQ comprises a mutant AAV2 inverted terminal repeat (ITR) and a wild type AAV2 ITR that enable packaging of self-complementary AAV genomes.

The scAAV9 was produced by transient transfection procedures using a double-stranded AAV2-ITR-based vector, with a plasmid encoding Rep2Cap9 sequence as previously described [Gao et al., J. Virol., 78: 6381-6388 (2004)] along with an adenoviral helper plasmid pHelper (Stratagene, Santa Clara, Calif.) in 293 cells. Virus was produced in three separate batches for the experiments and purified by two cesium chloride density gradient purification steps, dialyzed against PBS and formulated with 0.001% Pluronic-F68 to prevent virus aggregation and stored at 4° C. All vector preparations were titered by quantitative PCR using Taq-Man technology. Purity of vectors was assessed by 4-12% sodium dodecyl sulfate-acrylamide gel electrophoresis and silver staining (Invitrogen, Carlsbad, Calif.).

scAAV9 viruses were generated and titered by the Viral Vector Core at The Research Institute at Nationwide Children's Hospital.

### Example 6

### Neonatal Delivery of scAAV9.mi.P278KY and scAAV9.mi.ΔETAQ

To first establish the proof-of-principle of this approach in vivo, we tested whether the reduction of mutant GARS expression before disease onset could prevent the onset of neuropathy in GARS^{ΔETAQ/huEx8} mice. A total dose of ˜2.6×10¹¹ vg of scAAV9.mi.ΔETAQ or scAAV9.mi.LacZ (expressing a control microRNA targeting the E. coli LacZ gene) were delivered with an intracerebroventricular (ICV) injection at postnatal day 0-1 (P0-1) to GARS^{ΔETAQ/huEx8} and littermate control (GARS E8h) pups.

Prior to all injections of mice at P0-P1, all pups were anesthetized via cryoanesthesia. Once properly anesthetized, all intracerebroventricular injections were performed using a Hamilton syringe (cat no. 65460_03) with a 32-gauge needle. All gene therapy vectors were injected in to the lateral ventricles by positioning the needle directly lateral to the sagittal suture and rostral to the neonatal coronal suture. For intravenous injections, all cyroanesthetized mice were injected with 1×10¹¹ DRPS/mouse directly into the superficial temporal vein in a caudal orientation with a use of a Hamilton syringe (cat no. 7655-01) with a 32-gauge needle.

All mice were evaluated for established signs of neuropathy at 4-weeks-of-age, ˜1.5 weeks after the initial onset of overt signs of neuropathy. GARS^{ΔETAQ/huEx8} mice treated with scAAV9.mi.ΔETAQ showed significant improvement in a wire hang test of grip strength, increased muscle to body weight ratios (MW:BW), and improved sciatic nerve conduction velocity (NCV) compared to control-treated ΔETAQ mice (FIG. 4C-E). Examination of cross-sections of the motor branch of the femoral nerve revealed that scAAV9.mi.ΔETAQ treatment prevented the axon loss and lessened the decrease in axon diameters observed in untreated and vehicle control-treated ΔETAQ mice (FIG. 4F-H). Importantly, injection with scAAV9.mi.ΔETAQ (or scAAV9.LacZ) did not cause adverse effects in control mice by any of these outcome measures or by observation for overt reactions. Collectively, these data show that allele-specific knockdown of mutant ΔETAQ GARS expression prior to disease onset has a significant therapeutic effect and almost completely prevents behavioral, physiological, and histological signs of neuropathy, providing proof-of-concept data that allele-specific knockdown using virally delivered RNAi may be an effective approach for treating CMT2D.

### Example 7

### Intrathecal Delivery of Gene Therapy Constructs to Post-Onset Mice

To demonstrate the translational nature of the strategy, scAAV9.mi.ΔETAQ were delivered to cohorts of both early- (5-week-old) and late- (9-week-old) symptomatic GARS^{ΔETAQ/huEx8} mice and littermate controls via a single intrathecal (IT) injection into the lumbar spinal cord. With the use of a Hamilton syringe (cat no. 7655-01) with a 32 gauge needle, all adult post-onset mice were injected with ˜1×10¹¹ DRPS/mouse of scAAV9.mi.P278KY or scAAV9.mi.ΔETAQ diluted into sterile phosphate buffer saline (˜10 μls) with an intrathecal injection by lumbar puncture. Here, all mice were anesthetized with isoflurane and received an injection of the proper vector into the L6 spinous process with the use of a Hamilton syringe with a 32-gauge needle. Each vector was slowly injected and the needle left in place for 5-10 seconds prior to withdrawal.

When left untreated, 5-week old early symptomatic GARS^{ΔETAQ/huEx8} mice undergo active axon loss, while axon loss slows and muscle atrophy accelerates in 9-week old late symptomatic ΔETAQ mice.

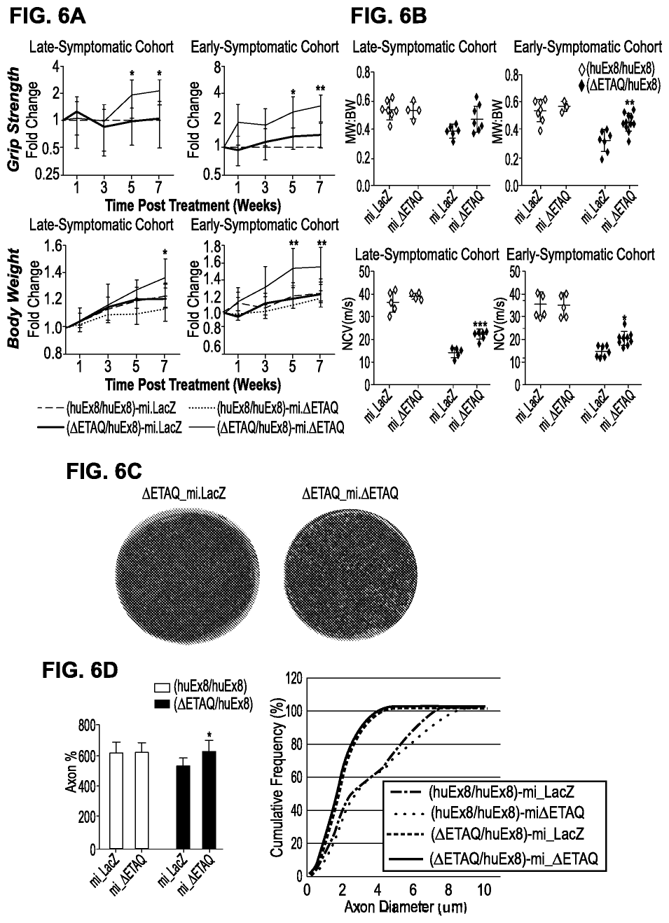

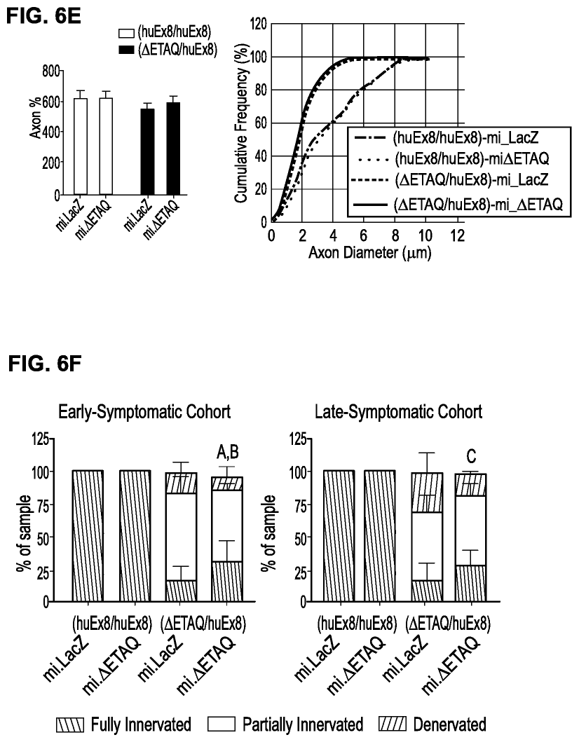

The scAAV9.mi.ΔETAQ-treated early-symptomatic GARS^{ΔETAQ/huEx8} mice displayed enhanced grip strength and significant increases in body weight starting at ˜5 weeks post treatment compared to untreated controls (FIG. 6A). When analyzed for primary signs of neuropathy at 7 weeks post treatment, early-symptomatic GARS^{ΔETAQ/huEx8} mice also exhibited significant increases in MW:BW ratios, nerve conduction velocity, NMJ innervation and a reduction in axon loss, but no improvement in axon size compared to untreated GARS^{ΔETAQ/huEx8} mice (FIG. 6B-D, F). When treated with scAAV9-mi.ΔETAQ at 9 weeks of age, GARS^{ΔETAQ/huEx8} mice gained weight and displayed enhanced grip strength starting at 5-7 weeks post treatment (FIG. 6A). While MW:BW ratios were not improved and axon loss and atrophy were not prevented, scAAV9.mi.ΔETAQ did improve nerve conduction velocity and NMJ innervation (FIG. 6B, E-F).

Statistical tests were performed using GraphPad's Prism 7 software. A two-tailed t test, one-way or two-way ANOVA followed Tukey's HSD posthoc comparisons test (as indicated in Figure legends) was used to determine significant differences between treatment and/or genotypes for axon counts, conduction velocity, grip strength, and body weight. Axon diameters were compared using non-parametric Kolmogorov-Smirnov two-sample and Shapiro-Wilk normality tests. NMJ innervation status between genotypes and categories (fully innervated, partially innervated, and denervated) was evaluated with a two-way ANOVA followed by Tukey's HSD posthoc comparisons test.

Whole liver and lumbar dorsal root ganglia samples were isolated from animals immediately after they were euthanized by cervical dislocation. The tissues were frozen in liquid nitrogen and stored at −80′C. Samples were homogenized using a mortar and pestle followed by a Dounce homogenizer and RNA was isolated from liver using Trizol Reagent (ThermoFisher, cat no. 15596018) and dorsal root ganglia using either a RNeasyMini Kit (Qiagen, cat nos. 74104 and 74106) or mirVana™ miRNA Isolation Kit (ThermoFisher Scientific, cat no. AM1560). All RNA samples were reverse transcribed using SuperScript™ Ill First-Strand Synthesis System (cat no. 18080051). To quantify allele-specific expression of wildtype and mutant GARS, EpigenDx (http://epigendx.com/d/) performed pyrosequencing on the PSQ96 HS System (Qiagen) following the manufacturer's instructions, using custom assays. Analysis of mRNA from sensory dorsal root ganglia (DRGs), which were also transduced by scAAV9, via pyrosequencing indicated that mutant GARS mRNA levels were significantly reduced in scAAV9.mi.ΔETAQ treated mice. See Table 1 below.

TABLE 1
Effects of scAAV9.mi.ΔETAQ on In Vivo GARS Expression in
Doral Root Ganglia

| | | | Average Ratio of Mutant:Wildtype |
|---|---|---|---|
| Age at Injection | Genotype | Treatment | GARS Expression (±SD) |
| Neonate (P0-P1) | (huEx8/huEx8) | Untreated | 14.0:86.0 (±3.28) |
| Neonate (P0-P1) | (huEx8/huEx8) | sc.AAV9.mi.LacZ | 8.0:92.0 (±1.275) |
| Neonate (P0-P1) | (huEx8/huEx8) | sc.AAV9.mi.ΔETAQ | 9.6:90.4 (±2.07) |
| Neonate (P0-P1) | (ΔETAQ/huEx8) | Untreated | 62.4:37.6 (±0.94) |
| Neonate (P0-P1) | (ΔETAQ/huEx8) | sc.AAV9.mi.LacZ | 62.5:38.6 (±0.96) |
| Neonate (P0-P1) | (ΔETAQ/huEx8) | sc.AAV9.mi.ΔETAQ | 46.9:53.1 (±4.79) |
| 5 Weeks | (huEx8/huEx8) | sc.AAV9.mi.LacZ | 9.6:90.4 (±2.53) |
| 5 Weeks | (huEx8/huEx8) | sc.AAV9.mi.ΔETAQ | 8.3:91.7 (±2.92) |
| 5 Weeks | (ΔETAQ/huEx8) | sc.AAV9.mi.LacZ | 63.9:36.1 (±3.13) |
| 5 Weeks | (ΔETAQ/huEx8) | sc.AAV9.mi.ΔETAQ | 47.2:52.8 (±7.88) |
| 9 Weeks | (huEx8/huEx8) | sc.AAV9.mi.LacZ | 10.3:89.7 (±3.04) |
| 9 Weeks | (huEx8/huEx8) | sc.AAV9.mi.ΔETAQ | 6.0:94.0 (±1.33) |
| 9 Weeks | (ΔETAQ/huEx8) | sc.AAV9.mi.LacZ | 59.3:40.7 (±6.45) |
| 9 Weeks | (ΔETAQ/huEx8) | sc.AAV9.mi.ΔETAQ | 45.3:54.74 (±6.02) |

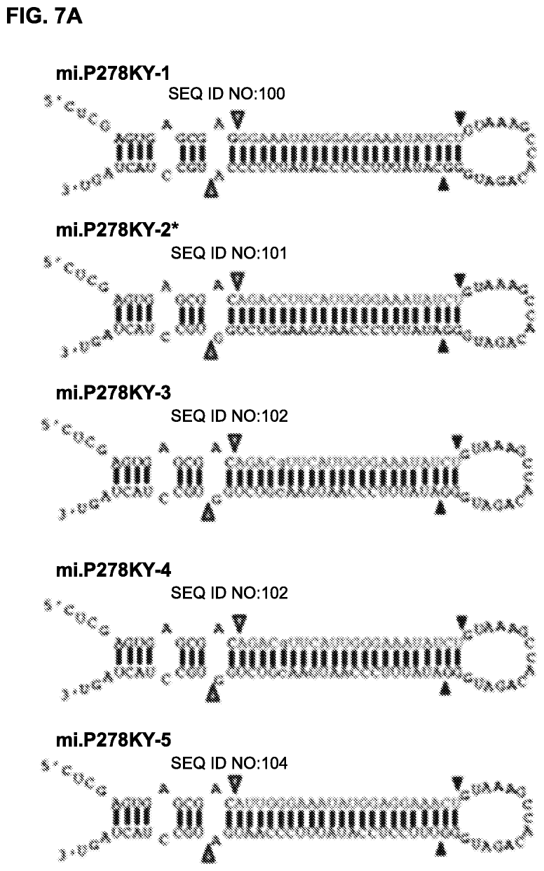

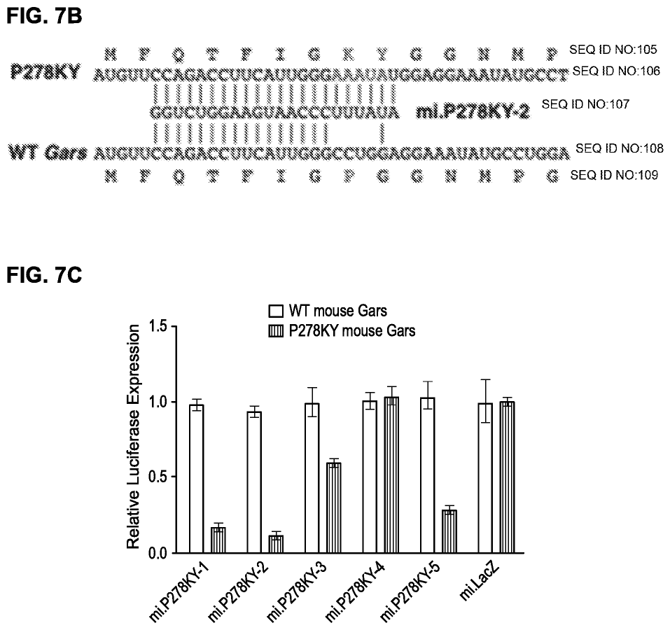

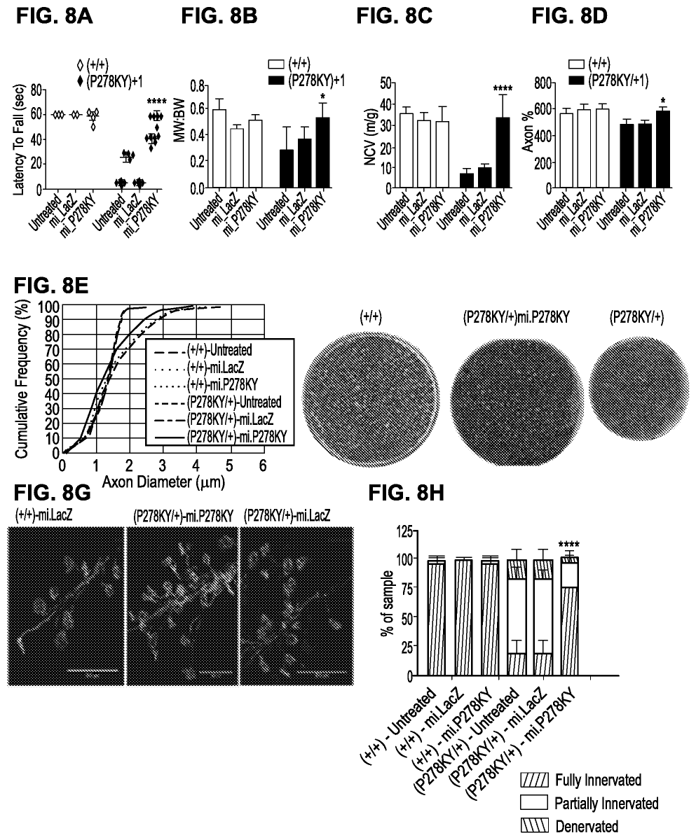

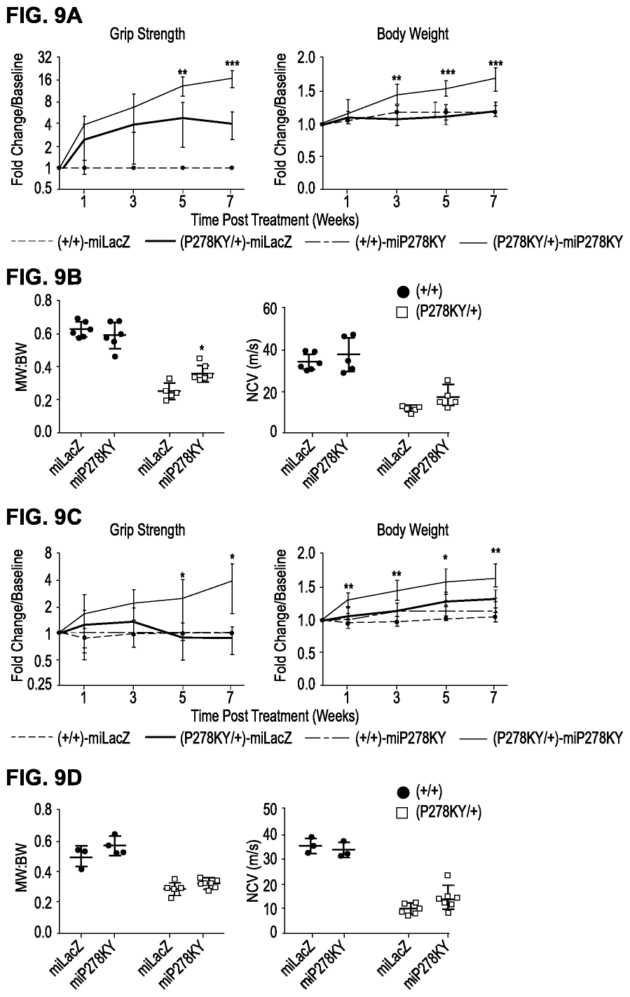

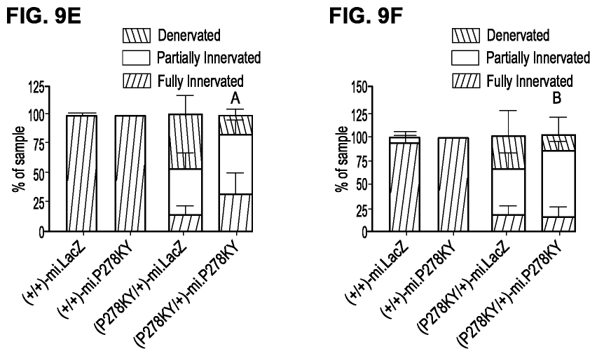

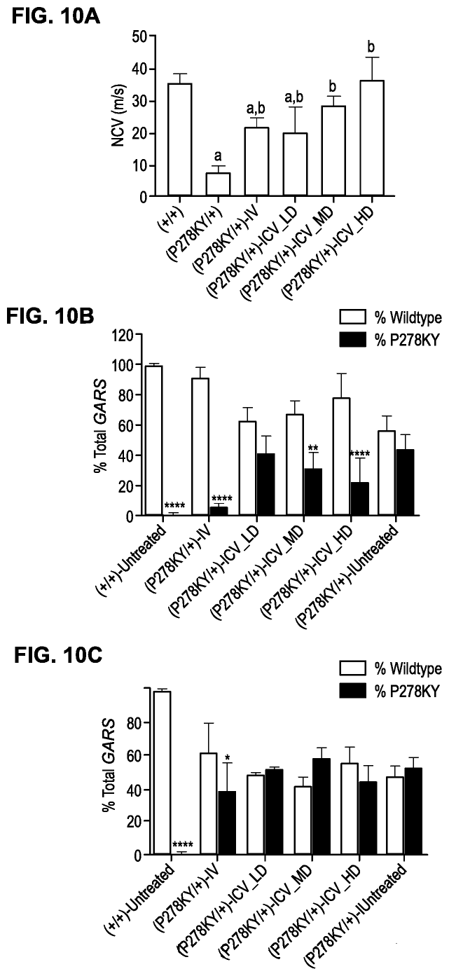

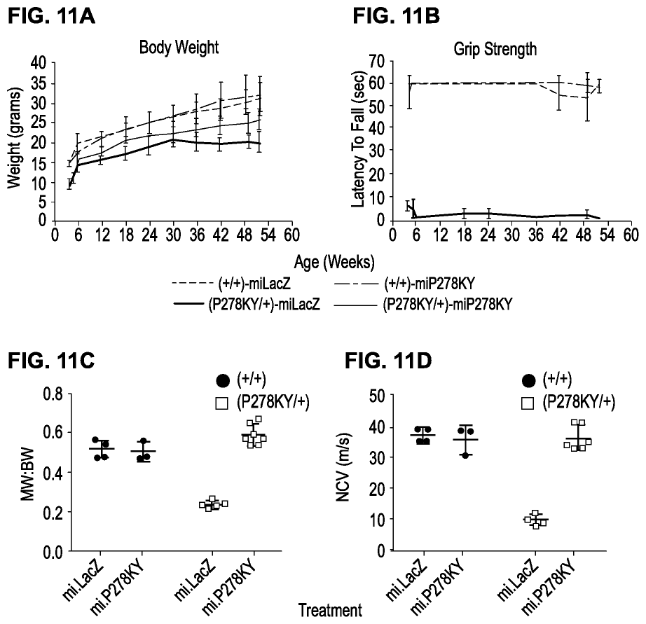

To establish that our approach would be generally applicable for CMT2D, we confirmed its efficacy in the second mouse model of CMT2D, GARS^{P278KY/+}. A miRNA shuttle targeting the mouse P278KY allele was optimized in our luciferase assay and packaged into scAAV9, as before (FIG. 7). Similar improvements were observed in neonatal, early- and late-symptomatic GARS^{P278KY/+} mice that were treated with 1×10¹¹ viral genomes of scAAV9.mi.P278KY delivered by ICV (neonates) or IV (adults) injection (FIG. 8, 9). Furthermore, the therapeutic effects of scAAV9.mi.P278KY were dose-dependent, and were greater with ICV delivery compared to a systemic, intravenous injection delivering the same total dose (FIG. 10). The beneficial effects of ICV delivery of the high dose (1×10¹¹ v.g.) of scAAV9.mi.P278KY at P0 lasted at least one year (FIG. 11).

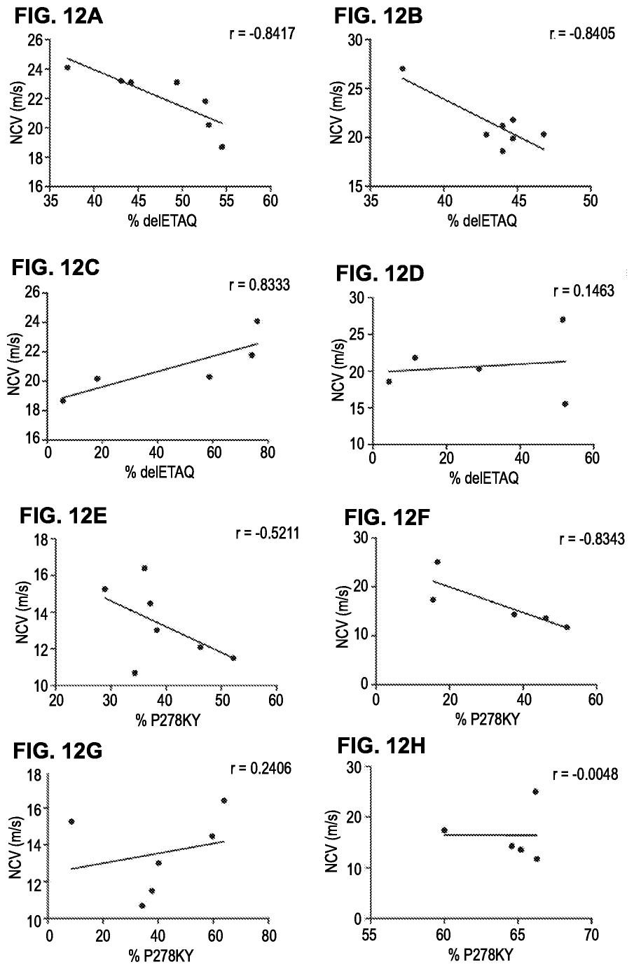

The knockdown efficacy of mutant GARS transcripts within dorsal root ganglion (Tables 1-3) strongly correlated with therapeutic outcomes within both post-onset studies (FIG. 12A-B, E-F).

TABLE 2
Effects of scAAV9.miP278KY on Allele-Specific GARS Expression in Neonates

| | | | Average Ratio of |
|---|---|---|---|
| | | | Mutant:Wildtype GARS |
| Genotype | Treatment | Dose:Route of Delivery | Expression (±SD) |
| (+/+) | Untreated | NA | 1.2:98.8 (±0.43) |
| (+/+) | sc.AAV9.mi.LacZ | 1 × 10¹¹ DRPS/animal:ICV | 1.0:99.0 (±0.32) |
| (+/+) | sc.AAV9.mi.P278KY | 1 × 10¹¹ DRPS/animal:ICV | 0.2:99.8 (±0.22) |
| (P278KY/+) | Untreated | NA | 55.8:44.2 (±9.93) |
| (P278KY/+) | sc.AAV9.mi.LacZ | 1 × 10¹¹ DRPS/animal:ICV | 51.7:48.3 (±8.46) |
| (P278KY/+) | sc.AAV9.mi.P278KY | 1 × 10¹¹ DRPS/animal:ICV | 22.2:77.8 (±16.44) |
| (P278KY/+) | sc.AAV9.mi.P278KY | 5 × 10¹⁰ DRPS/animal:ICV | 33.3:66.65 (±9.1 |
| (P278KY/+) | sc.AAV9.mi.P278KY | 8.75 × 10⁹ DRPS/animal:ICV | 38.5:62.15 (±8.91) |
| (P278KY/+) | sc.AAV9.mi.P278KY | 1 × 10¹¹ DRPS/animal:IV | 9.8:90.2 (±8.22) |

ICV = Intracerebroventricular Delivery
IV = Intravenous Delivery
NA = Not Available

TABLE 3
Post-Onset Effects of scAAV9.mi.P278KY on In Vivo GARS
Expression in Doral Root Ganglia

| | | | Average Ratio of |
|---|---|---|---|
| Age at | | | Wildtype:Mutant GARS |
| Injection | Genotype | Treatment | Expression (±SD) |
| 5 Weeks | (+/+) | scAAV9.mi.LacZ | 1.6:98.4 (±2.46) |
| 5 Weeks | (+/+) | sc.AAV9.mi.P278KY | 0.7:99.3 (±0.55) |
| 5 Weeks | (P278KY/+) | sc.AAV9.mi.LacZ | 55.0:45.0 (±7.26) |
| 5 Weeks | (P278KY/+) | sc.AAV9.mi.P278KY | 36.2:63.8 (±14.74) |
| 9 Weeks | (+/+) | sc.AAV9.mi.LacZ | 0.9:99.1 (±0.27) |
| 9 Weeks | (+/+) | sc.AAV9.mi.P278KY | 0.0:100.0 (±0.00) |
| 9 Weeks | (P278KY/+) | sc.AAV9.mi.LacZ | 52.3:47.7 (±4.64) |
| 9 Weeks | (P278KY/+) | sc.AAV9.mi.P278KY | 40.6:59.4 (±8.95) |

This correlation was stronger with DRGs than when outcomes were compared to mutant GARS mRNA levels in liver, another peripheral tissue transduced by scAAV9 (FIG. 12C-D, G-H). This is consistent with mutant forms of GARS causing neuropathy through a cell-autonomous mechanism.

These data indicate allele-specific knockdown as a general therapeutic strategy for patients with CMT2D. Taken together, these data confirm that allele-specific RNAi-based gene therapy can improve symptoms of neuropathy in mouse models of CMT2D-including a “humanized” model- and even at post-onset phases of the disease.

### Example 8

### rAAV9-miGARS/rGARS Vector and Use

A rAAV9 (rAAV9-miGARS/rGARS vector) that knocks down mutant and wild-type Gars expression with RNAi, and also restores wild-type Gars expression with an RNAi-resistant cDNA (rGARS) is generated. There are two key components in the vector: (1) a cassette encoding GARS-targeted microRNA; (2) an RNAi-resistant replacement human GARS cDNA cassette (2.2 kb). The total size of the payload, including the AAV inverted terminal repeats (ITRs) is ˜4.0 kb, thereby necessitating use of ssAAV vectors. The two cassettes will be cloned side-by-side in head-to-tail orientation (where promoter is 5′ end, and terminator or poly A is 3′).

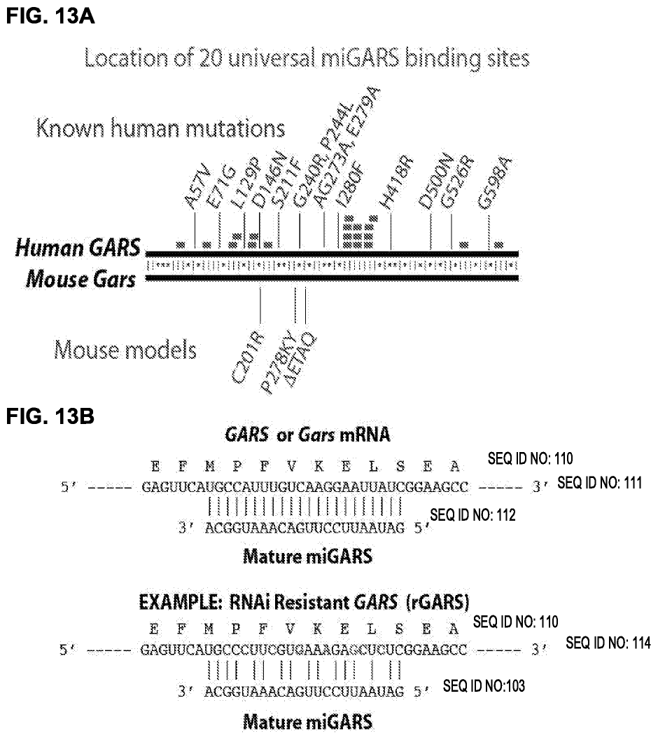

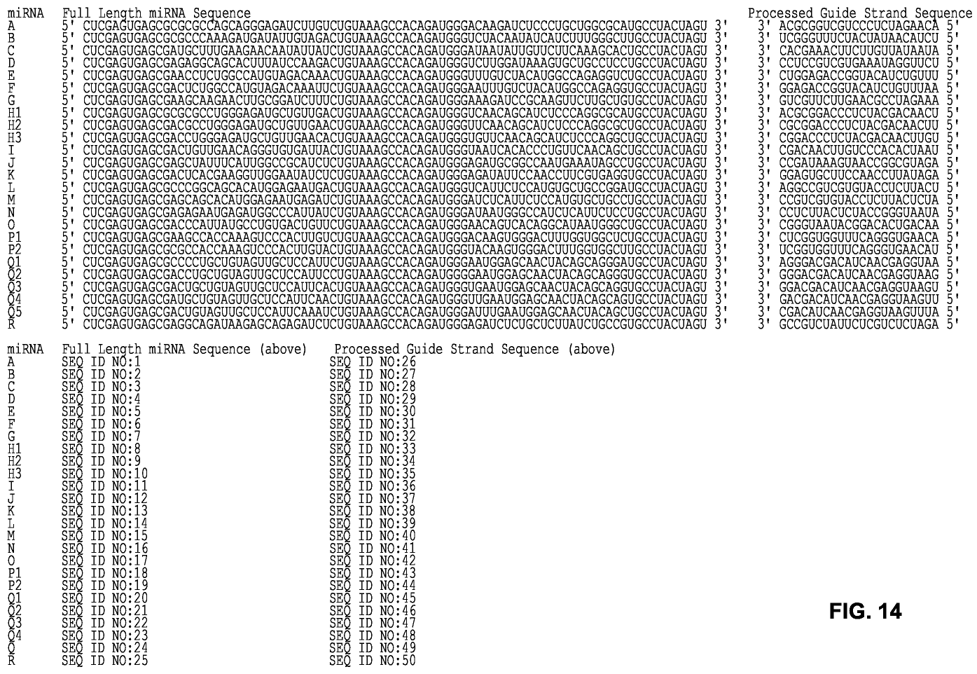

Artificial miRNAs are based on the natural mir-30, maintaining important structural and sequence elements required for normal miRNA biogenesis but replacing the mature mir-30 sequences with 22-nt of perfect complementarity with the GARS gene. In addition, the “miGARS” target the common regions between the mouse and human GARS gene, while avoiding any sequences containing known GARS mutations associated with CMT2D. See FIG. 13A. This design strategy provides two major advantages: (1) non-allele specific GARS gene silencing and (2) testing for efficacy in mice with direct translatability in humans. The miGARS are transcribed from the U6 promoter. The miRNA expression cassette is ˜500 bp in size. Sequences of the miGARS are shown in FIG. 14.

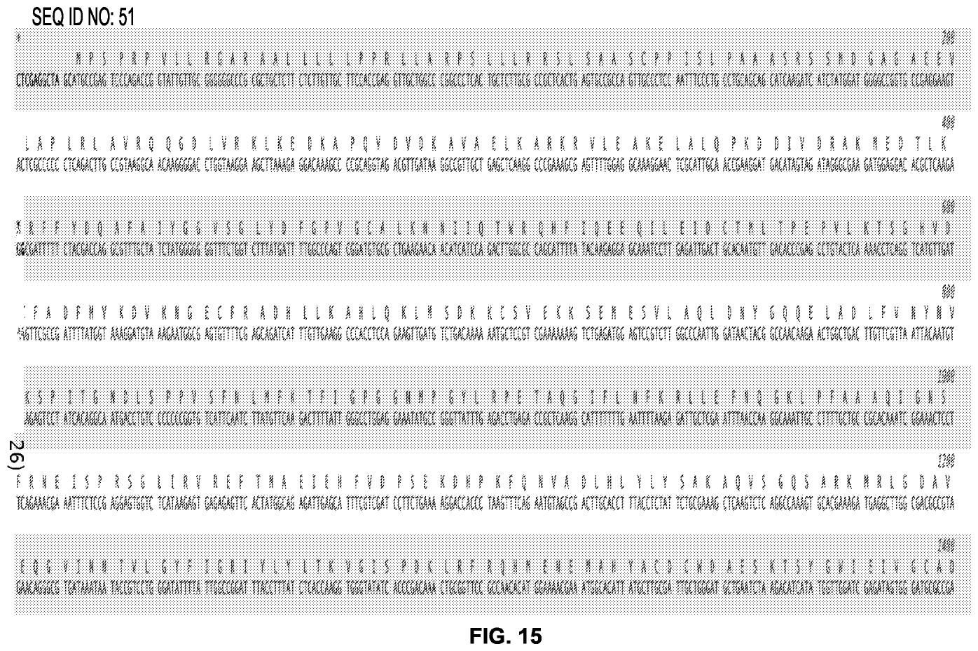

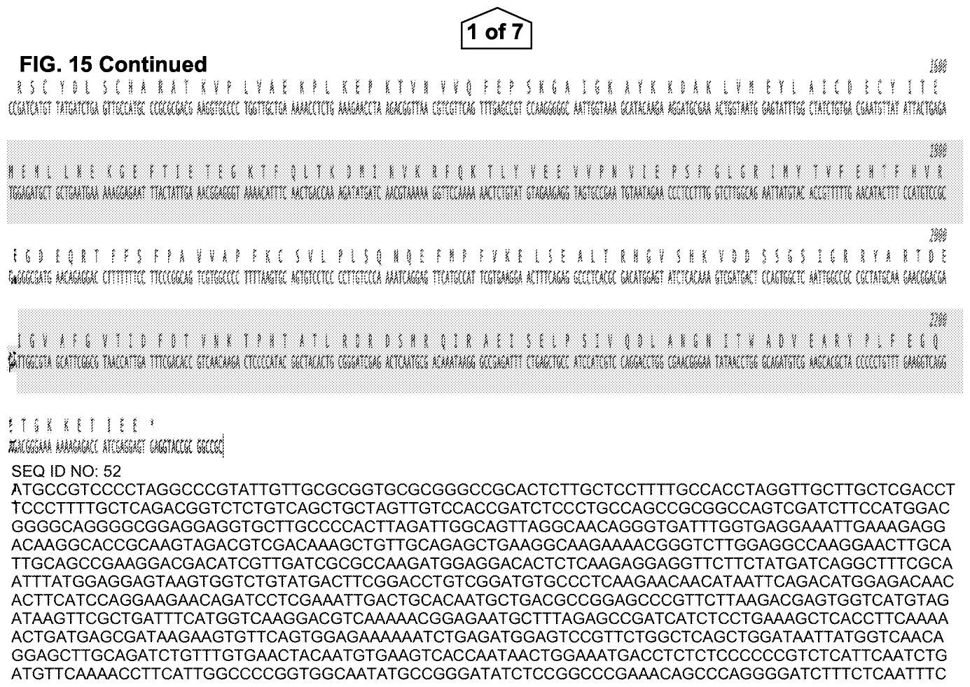

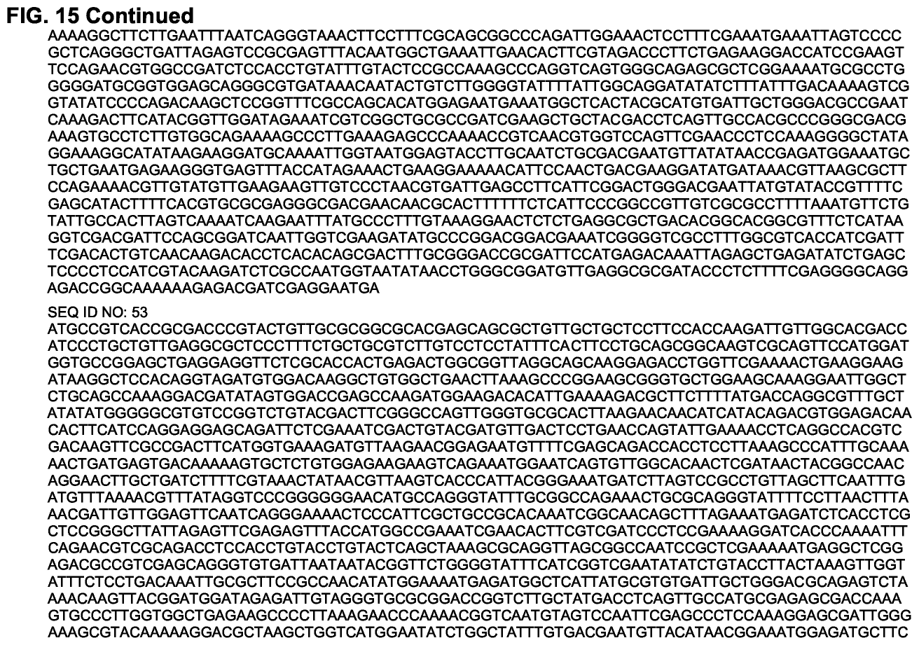

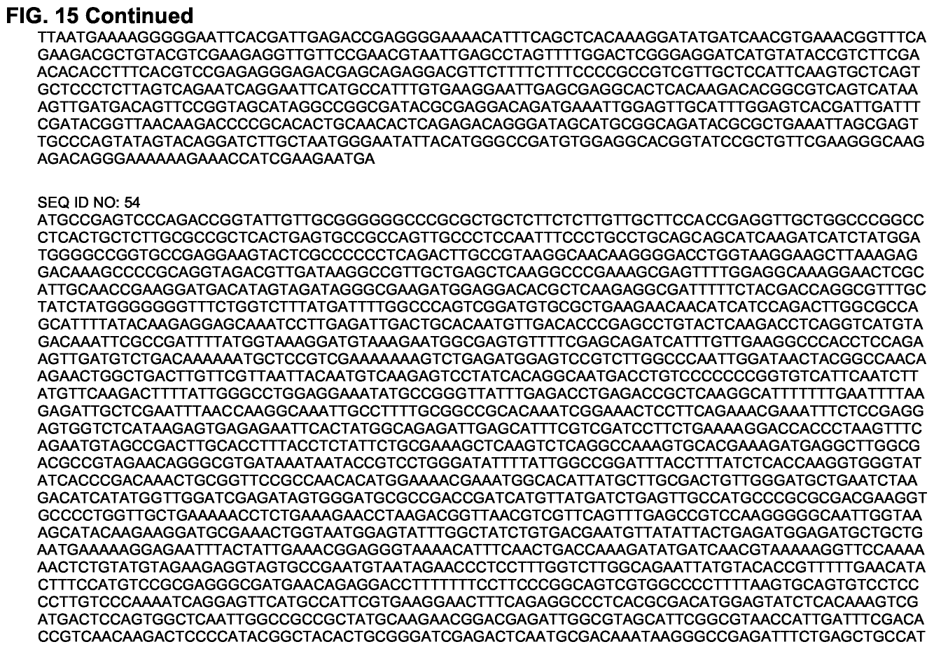

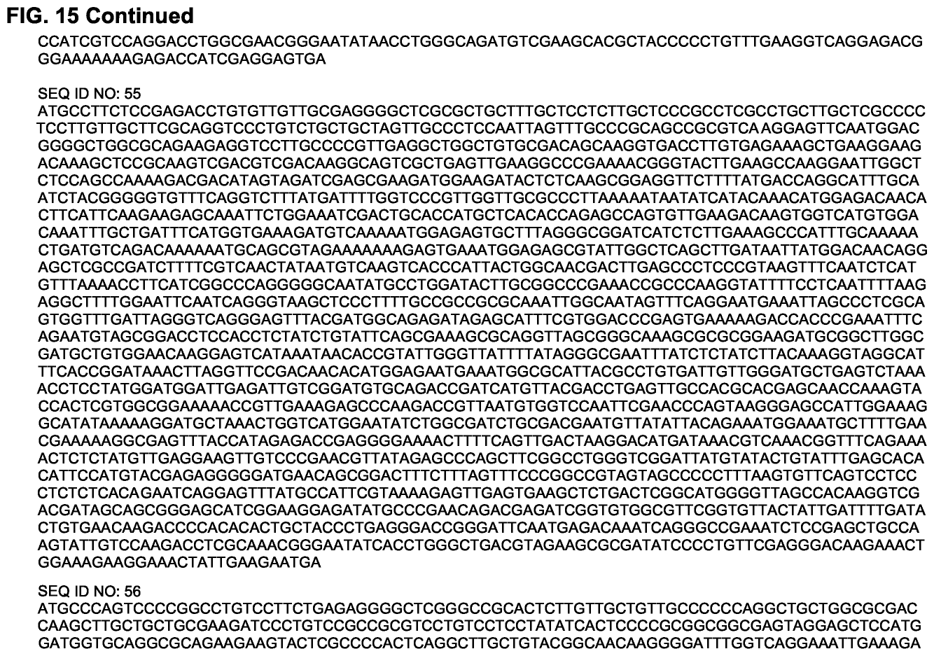

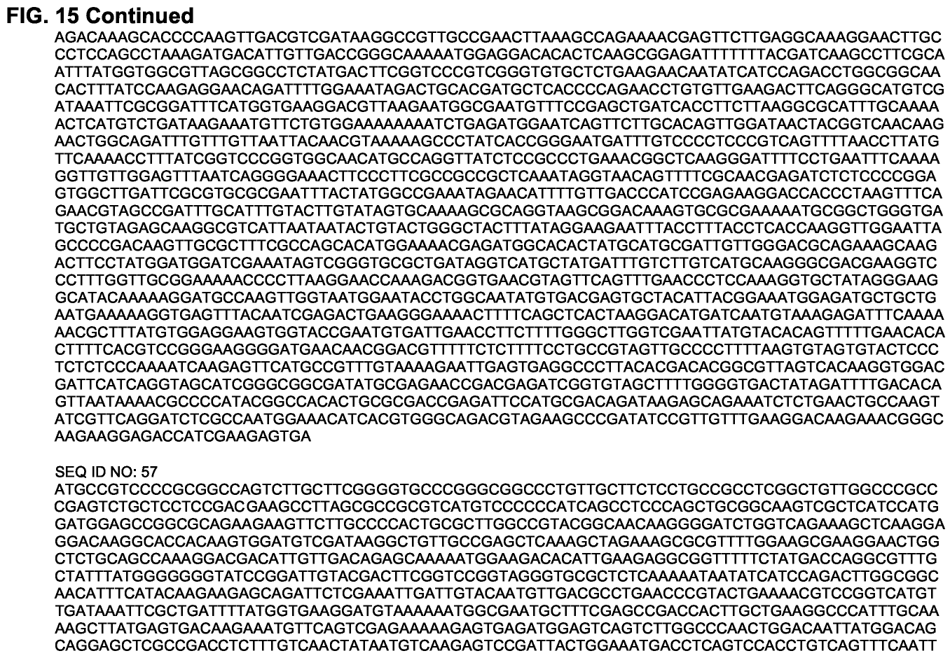

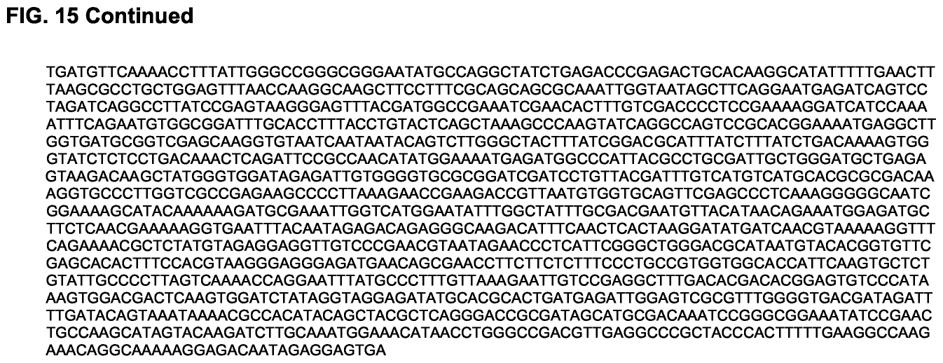

The ˜2.2-kb full-length human GARS cDNA is modified to render it resistant to knockdown by the miGARs. To do this, nucleotide wobble positions in the cDNA within the binding site of the miGARS are mutated, without changing the wild-type GARS amino acid sequence that is encoded by the cDNA. See FIG. 13B. Exemplary cDNA sequences are shown in FIG. 15. RNAi-resistant GARS cDNA (called “rGARS”) will be transcribed from the 800-bp chicken β-actin (CBA) promoter. A ˜200-bp SV40 polyA signal will be placed at the 3′ end of the ORF. The total size of the rGARS expression cassette is 3,200-bp. A short, artificial 3′ UTR will be attached to allow specific detection of rGARS, distinguishing it from endogenous mouse or human GARS alleles.

The GARS CMT2D ΔETAQ mouse model is dosed with the rAAV9-miGARS/rGARS vector at the maximally effective dose at two time points, pre-onset (ICV at P0) and post onset (intrathecal). P278KY and C201R mice can also be dosed. Cohort sizes will be ˜20 mice per group. Groups will include Gars mutant mice and littermate controls randomly assigned to treatment, negative control (AAV9-LacZ) and positive control (allele-specific knockdown vector for ΔETAQ) groups. Mice will be monitored longitudinally with behavioral tests (grip strength), noninvasive NCV/EMG, and body weight. One cohort of mice for each genotype will be analyzed in detail 1 to 2 months after treatment to show short-term efficacy. A second cohort will be allowed to age to determine the endurance of the effects. Terminal outcome measures include nerve histology, neuromuscular junction analysis, and muscle weights and/or muscle histology.

### Example 9

### Target GARS Replacement Activity Level

Experiments were performed to define the lower limit of wild type Gars gene expression required for normal function.

CMT2D is an autosomal dominant disease in both human patients and mice. Therefore, specifically reducing the mutant allele while leaving the wild-type allele unperturbed results in half the normal amount of GARS gene expression. Although homozygous GARS null mice die as embryos, heterozygous Gars^{+/null} mice display a normal phenotype, demonstrating that there is a level of GARS activity less than 100 percent that is sufficient for a normal phenotype.

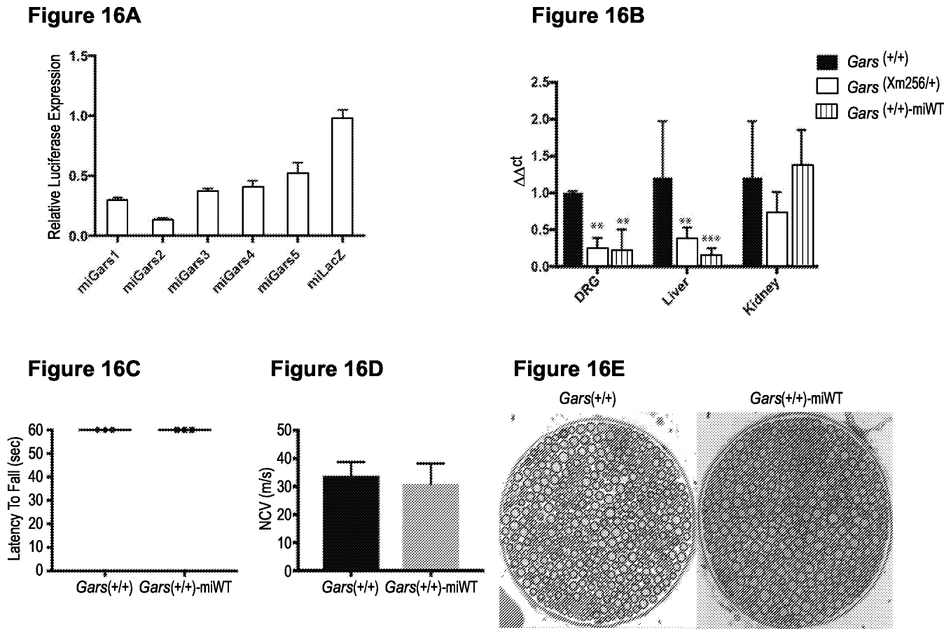

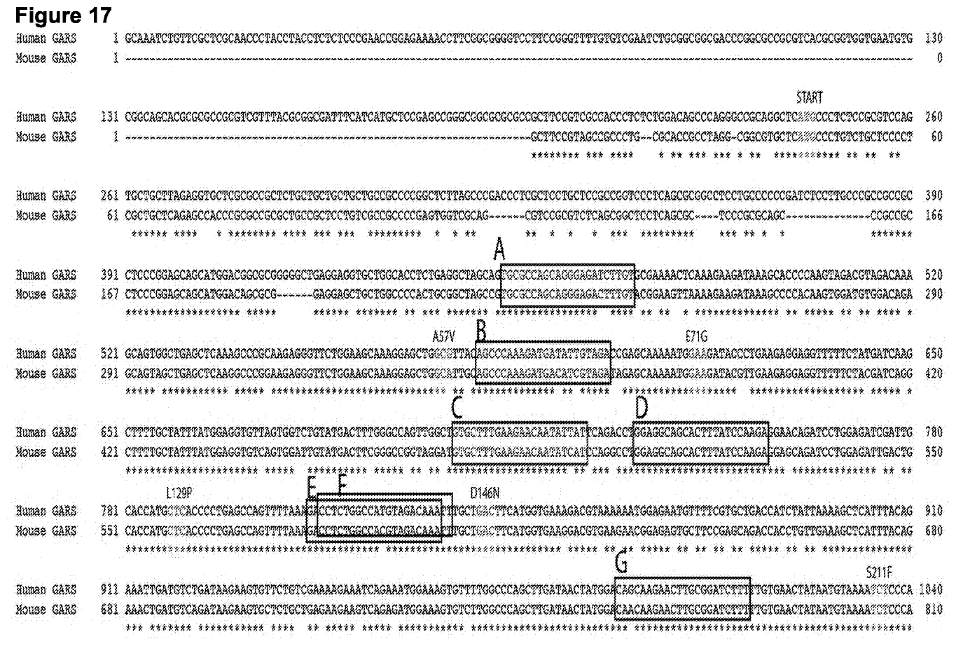

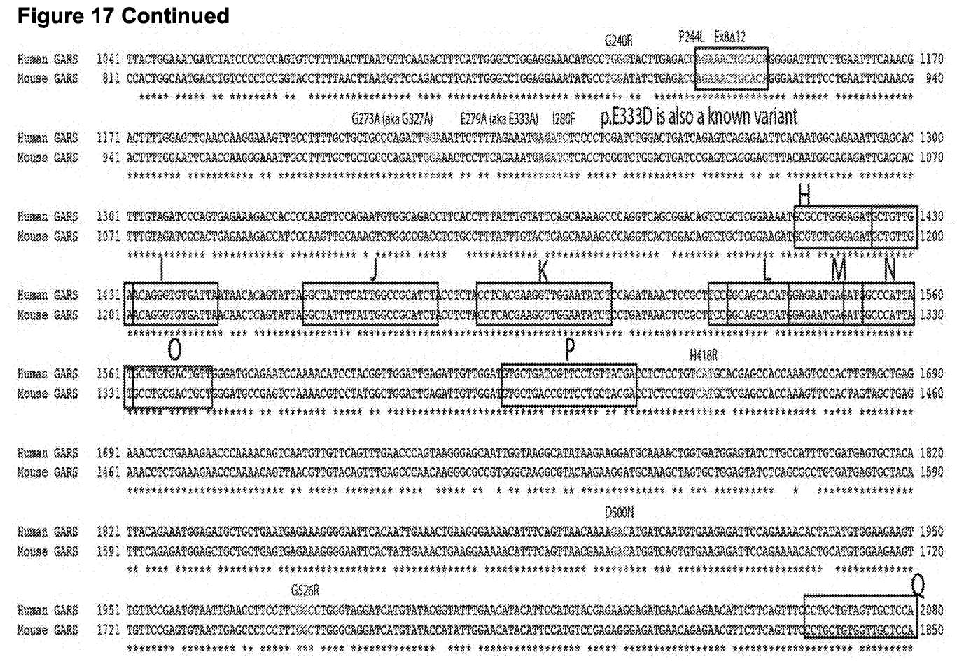

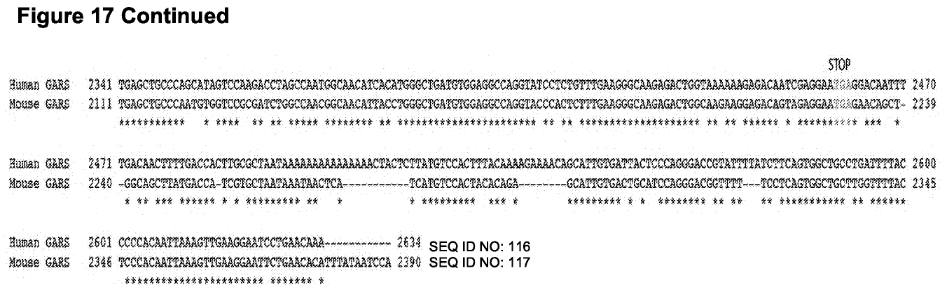

To determine the lower limit of wild-type GARS activity sufficient for a normal phenotype, AAV9 vectors carrying microRNAs targeting wild-type mouse GARS (miWT) (FIG. 16A) were delivered to neonatal GARS^{+/+} and GARS^{null/+} mice. Although, miWT-treated GARS^{null/+} mice died within 24 hours after treatment, ˜70% reduction in total GARS expression within tissues transduced by the scAAV9 was tolerated by GARS^{(+/+)} mice throughout their development (FIG. 16B).

To confirm that such a reduction in total GARS did not cause neuropathy, adult miWT-treated GARS^{(+/+)} were analyzed for signs of neuropathy including possible reductions in grip strength and nerve conduction velocity as well as axon atrophy. Remarkably, at 12-weeks-of-age, an age in which the onset of neuropathy occurs in all established mouse models of CMT2D, miWT-treated GARS^{(+/+)} were phenotypically normal and did not did display any signs of axon degeneration (FIG. 16C-D).

Thus, these data indicate that ˜30% of wild-type GARS expression is sufficient for a normal phenotype. In addition, these data support that GARS-associated CMT2D is caused by a toxic gain-of-function or dominant negative mechanism(s) and not just loss of canonical GARS activity.

### Example 10

### ΔETAQ GARS Affects the Primary Function of the Enzyme

Experiments were performed to assess if ΔETAQ GARS affects the primary function of the enzyme. Both aminoacylation assays and yeast complementation tests were carried out.

For aminoacylation assays, wild-type and mutant GARS proteins were expressed in E. coli with a C-terminal His tag and purified with nickel affinity resins (Novagen). The T7 transcript of human tRNA^{Gly/CCC} (CCC, anticodon) was prepared and purified as previously described (Hou et al., Proc. Natl. Acad. Sci. USA 1993; 90(14):6776-80), heat denatured at 85° C. for 3 min, and annealed at 37° C. for 20 min before use. Steady-state aminoacylation assays were monitored at 37° C. in 50 mM HEPES (pH 7.5), 20 mM 28 KCl, 10 mM MgCl2, 4 mM DTT, 2 mM ATP, and 50 μM 3 H-glycine (Perkin Elmer) at a specific activity of 16,500 dpm/pmole. The reaction was initiated by mixing GARS enzyme (20 nM for WT enzyme and 600 nM for the ΔETAQ and P234KY mutants) with varying concentrations of tRNA (0.3-20 μM). Aliquots of a reaction mixture were spotted on filter paper, quenched by 5% trichloroacetic acid, washed, dried, and measured for radioactivity using a liquid scintillation counter (LS6000SC; Beckman Coulter Inc.). The amount of radioactivity retained on filter pads was corrected for quenching effects to determine the amount of synthesis of Gly-tRNA Gly. Steady-state kinetics was determined by fitting the initial rate of aminoacylation as a function of tRNA concentration to the Michaelis-Menten equation (Schreier et al., Biochemistry. 1972; 11(9):1582-9).

Yeast complementation assays were carried out using a haploid S. cerevisiae strain with the endogenous GRS1 locus deleted and viability maintained via a pRS316 vector expressing the-wild type GRS1 gene (Antonellis et al., J Neuroscience 2006; 26(41):10397-406., Turner et al., J. Biol. Chem. 2000; 275(36):27681-8). To assess the ability of wild-type and mutant GARS alleles to support cellular growth, the haploid yeast strain was transformed with wild-type or mutant constructs, or a construct bearing no GARS insert. Transformed yeast cells were selected for the presence of both the maintenance and experimental vectors by growth on solid media lacking leucine and uracil. Colonies were grown to saturation in 2 mL liquid medium lacking leucine and uracil at 30° C., 275 rpm for 48 hours. Undiluted cultures and dilutions of 1:10 and 1:100 were spotted on complete solid medium containing 0.1% 5-FOA (Teknova, Hollister Calif.); 5-FOA selects for cells that have spontaneously lost the maintenance vector (Boeke et al., Mol Gen Genet. 1984; 197(2):345-6). Yeast viability was assessed after 4 days of incubation at 30° C. At least two colonies per transformation were assayed and each transformation was repeated at least twice.

Analysis of the initial rate of aminoacylation as a function of the tRNA substrate concentration showed that ΔETAQ GARS retained less than 0.01% aminoacylation activity compared to wild-type GARS indicating that it is a functional null allele. In parallel, the previously described mouse allele, P234KY (P278KY in the mouse, where 234 is numbered without the 44 amino acid mitochondrial targeting sequence included), was tested, given its nearby location in the protein (Seburn et al. Neuron. 2006; 51(6):715-26). Although the P234KY allele showed activity in assays with saturating tRNA and glycine substrate concentrations (Seburn, supra), a re-evaluation of kinetic properties under Michaelis-Menton conditions showed a marked decrease in enzyme activity, making ΔETAQ GARS highly analogous to P234KY GARS. The reduced function of the ΔETAQ allele was further supported by the failure of this mutant protein to complement ablated cellular growth associated with deletion of the yeast ortholog GRS1. Data from this latter assay also support the LoF effect associated with P278KY GARS, and is consistent with the failure of the mouse P278KY allele to complement an RNA-null allele of Gars (Seburn, supra).

### Example 11

### ΔETAQ GARS Showed Slightly Aberrant Interaction with NRP1

Neuropathy-associated GARS mutations cause inappropriate binding to neuropilin-1 (NRP1), which leads to impaired NRP1/VEGF signaling in motor neurons (23). To directly test for binding between ΔETAQ GARS and NRP1, V5-tagged wild-type, P234KY, and ΔETAQ GARS were expressed in the mouse motor neuron cell line NSC-34.

The NSC-34 cell line was purchased from ATCC and cultured under standard conditions. Cells were grown to 70% confluency before transfection. A human wild-type, P234KY, or ΔETAQ GARS cDNA was cloned into the pcDNA6 plasmid to express GARS in-frame with a V5 tag. Transfections were performed using Lipofectamine 2000 (Invitrogen) according to the manufacturer's instruction. For cell lysate preparations, NSC-34 cells (36 hours after transfection) were washed twice in phosphate-buffered saline (PBS), scraped into PBS, pelleted, and resuspended in Pierce IP Lysis Buffer (Thermo Scientific) for 30 min and centrifuged for 7 min at 12,000×g; the insoluble fraction was discarded. Protein G beads (Invitrogen) were pre-incubated with anti-NRP1 antibody (Abcam) or rabbit IgG (Cell signaling) for 30 min before mixed with the cell lysates for overnight. Beads were then washed 3× with buffer (100 mM NaCl, 50 mM Tris, pH 7.5, 0.1% Triton X-100, 5% glycerol). The immunoprecipitates were fractionated by 4-12% Bis-Tris-Plus SDS-PAGE gels (Invitrogen) and transferred to PVDF membranes using the iBlot Dry Blotting System (Invitrogen). Membranes were blocked for 1 hour with Tris Buffered Saline with Tween 20 (TBST) containing 5% nonfat dry milk. Wild-type and mutant GARS proteins were detected using mouse monoclonal V5 antibody purchased from Invitrogen. NRP1 was detected by utilizing the same antibody for co-immunoprecipitation. After incubation with primary antibodies, membranes were washed and incubated with HRP-conjugated anti-mouse or anti-rabbit secondary antibodies (Cell Signaling), followed by detection using ECL western blotting substrate (Thermo Scientific) and exposed using the FluorChem M imager (ProteinSimple).

After immunoprecipitation with an anti-NRP1 antibody, proteins were subjected to Western blot analysis using an anti-V5 antibody. In contrast to the strong V5 signal associated with P234KY GARS as reported (23), the V5 signal associated with ΔETAQ GARS was much weaker, although stronger than that of wild-type GARS, which showed no V5 signal. Nevertheless, no interaction between ΔETAQ GARS and NRP1 was detected in unbiased mass spectrometry analyses of proteins immuno-precipitated from mouse neuroblastoma (MN1) cells expressing FLAG-tagged wild-type or V5-tagged ΔETAQ GARS. In sum, ΔETAQ showed a severe defect in aminoacylation activity and at best a slightly aberrant interaction with NRP1.

While the present invention has been described in terms of specific embodiments, it is understood that variations and modifications will occur to those skilled in the art. Accordingly, only such limitations as appear in the claims should be placed on the invention.

All documents referred to in this application are hereby incorporated by reference in their entirety.

We claim:

1. A nucleic acid comprising
(a) a nucleic acid encoding a Glycyl-tRNA Synthetase (GARS) miRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-25;
(b) a nucleic acid encoding a GARS guide strand comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 26-50; or
(c) a nucleic acid encoding a GARS miRNA comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-25 and a nucleic acid comprising an RNAi-resistant GARS gene comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 51-57.

2. A viral vector comprising the nucleic acid of claim 1 or a combination of any one or more thereof.

3. The viral vector of claim 2, wherein the viral vector is an adeno-associated virus (AAV), adenovirus, lentivirus, retrovirus, poxvirus, baculovirus, herpes simplex virus, vaccinia virus, or a synthetic virus.

4. The viral vector of claim 3, wherein the viral vector is an AAV.

5. The viral vector of claim 4, wherein the AAV lacks rep and cap genes.

6. The viral vector of claim 4 or 5, wherein the AAV is a recombinant AAV (rAAV) or a self-complementary recombinant AAV (scAAV).

7. The viral vector of any one of claims 4-6, wherein the AAV has a capsid serotype selected from the group consisting of: AAV-1, AAV-2, AAV-3, AAV-4, AAV-5, AAV-6, AAV-7, AAV-8, AAV-9, AAV-10, AAV-11, AAV-12, AAV-13, AAV-anc80, and AAV rh.74.

8. The viral vector of any one of claims 4-7, wherein the AAV has a capsid serotype of AAV-9.

9. The viral vector of any one of claims 4-8, wherein the AAV is a pseudotyped AAV.

10. The viral vector of claim 9, wherein the AAV is AAV2/8 or AAV2/9.

11. The viral vector of any one of claims 4-10, wherein expression of the nucleic acid encoding the GARS miRNA is under the control of a U6 promoter.

12. The viral vector of any one of claims 4-10, wherein expression of the RNAi-resistant replacement GARS gene is under the control of a chicken β-actin promoter.

13. A composition comprising the nucleic acid of claim 1 and a pharmaceutically acceptable carrier.

14. A composition comprising the viral vector of any one of claims 2-12 and a pharmaceutically acceptable carrier.

15. A composition comprising a delivery vehicle capable of delivering agents to a neuronal cell and
(a) a nucleic acid comprising an RNAi-resistant human GARS gene;
(b) a nucleic acid encoding a miRNA, wherein the miRNA binds a segment of a messenger RNA (mRNA) encoded by a human Glycyl-tRNA Synthetase (GARS) gene, the segment is conserved relative to the wild-type mouse GARS gene, and the segment does not encode sequence comprising a mutation associated with CMT2D; or
(c) a combination of (a) and (b) and; optionally,
(d) a pharmaceutically acceptable carrier.

16. The composition of claim 16, wherein the nucleic acid comprising the RNAi-resistant human GARS gene comprises a polynucleotide comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence of any one of SEQ ID NOs: 51-57.

17. The composition of claim 15 or 16, wherein the human GARS gene comprises the sequence of SEQ ID NO: 69, or a variant thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99%, identity to the sequence of SEQ ID NO: 69.

18. The composition of any one of claims 15-17, wherein the mouse GARS gene comprises the sequence of SEQ ID NO: 70, or a variant thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99%, identity to the sequence of SEQ ID NO: 70.

19. The composition of any one of claims 15-18, wherein the mRNA segment is complementary to a sequence within nucleotides 136-323, 327-339, 544-590, 720-785, 996-1406, 1734-1913 or 1950-2187 of a human GARS gene comprising the sequence of SEQ ID NO: 69.

20. The composition of claim 19, wherein the mRNA segment is complementary to a sequence within nucleotides 996-1406 of SEQ ID NO: 69.

21. The composition of any one of claims 16-21, wherein the delivery vehicle is a viral vector.

22. The composition of claim 21, wherein the viral vector is an adeno-associated virus (AAV), adenovirus, lentivirus, retrovirus, poxvirus, baculovirus, herpes simplex virus, vaccinia virus, or a synthetic virus.

23. The composition of claim 22, wherein the viral vector is an AAV.

24. The composition of claim 23, wherein the AAV lacks rep and cap genes.

25. The composition of claim 23 or 24, wherein the AAV is a recombinant AAV (rAAV) or a self-complementary recombinant AAV (scAAV).

26. The composition of any one of claims 23-25, wherein the AAV has a capsid serotype selected from the group consisting of: AAV-1, AAV-2, AAV-3, AAV-4, AAV-5, AAV-6, AAV-7, AAV-8, AAV-9, AAV-10, AAV-11, AAV-12, AAV-13, AAV-anc80, and AAV rh.74.

27. The composition of any one of claims 23-26, wherein the AAV has a capsid serotype of AAV-9.

28. The composition of any one of claims 23-27, wherein the AAV is a pseudotyped AAV.

29. The composition of claim 28, wherein the AAV is AAV2/8 or AAV2/9.

30. The composition of any one of claims 21-29, wherein expression of the nucleic acid encoding the GARS miRNA is under the control of a U6 promoter.

31. The composition of any one of claims 21-29, wherein expression of the RNAi-resistant replacement GARS gene is under the control of a chicken β actin promoter.

32. A method of delivering to a neuronal cell comprising a mutant Glycyl-tRNA Synthetase (GARS) gene, the method comprising administering to the neuronal cell:
(a) the nucleic acid of claim 1;
(b) the vector of any one of claims 2-12; or
(c) the composition of any one of claims 13-31.

33. A method of treating a subject suffering from a mutant Glycyl-tRNA Synthetase (GARS) gene, the method comprising administering to the subject:
(a) the nucleic acid of claim 1;
(b) the vector of any one of claims 2-12; or
(c) the composition of any one of claims 13-31.

34. The method of claim 33 wherein the subject suffers from Charcot-Marie-Tooth Disease Type 2D (CMT2D) or Distal Hereditary Motor Neuropathy.

35. The method of claim 32, wherein the neuronal cell is a human neuronal cell.

36. The method of claim 33 or 34, wherein the subject is a human subject.

37. Use of at least one nucleic acid of claim 1, the viral vector of any one of claims 2-12, or the composition of any one of claims 13-31 in treating a subject suffering from a mutant Glycyl-tRNA Synthetase (GARS) gene.

38. Use of at least one nucleic acid of claim 1, the viral vector of any one of claims 2-12, or the composition of any one of claims 13-31 in treating Charcot-Marie-Tooth Disease Type 2D (CMT2D) or Distal Hereditary Motor Neuropathy in a subject in need thereof.


SEQUENCE LISTING (114 sequences)

SEQ ID NO: 1 - DNA, 86 bp, Artificial Sequence
  Synthetic Polynucleotide
cucgagugagcgcgcgccagcagggagaucuugucuguaaagccacagaugggacaagaucucccugcuggcgcaugccuacuagu

SEQ ID NO: 2 - DNA, 86 bp, Artificial Sequence
  Synthetic Polynucleotide
cucgagugagcgcgcccaaagaugauauuguagacuguaaagccacagaugggucuacaauaucaucuuugggcuugccuacuagu

SEQ ID NO: 3 - DNA, 86 bp, Artificial Sequence
  Synthetic Polynucleotide
cucgagugagcgaugcuuugaagaacaauauuaucuguaaagccacagaugggauaauauuguucuucaaagcacugccuacuagu

SEQ ID NO: 4 - DNA, 86 bp, Artificial Sequence
  Synthetic Polynucleotide
cucgagugagcgagaggcagcacuuuauccaagacuguaaagccacagaugggucuuggauaaagugcugccuccugccuacuagu

SEQ ID NO: 5 - DNA, 86 bp, Artificial Sequence
  Synthetic Polynucleotide
cucgagugagcgaaccucuggccauguagacaaacuguaaagccacagauggguuugucuacauggccagaggucugccuacuagu

SEQ ID NO: 6 - DNA, 86 bp, Artificial Sequence
  Synthetic Polynucleotide
cucgagugagcgacucuggccauguagacaaauucuguaaagccacagaugggaauuugucuacauggccagaggugccuacuagu

SEQ ID NO: 7 - DNA, 86 bp, Artificial Sequence
  Synthetic Polynucleotide
cucgagugagcgaagcaagaacuugcggaucuuucuguaaagccacagaugggaaagauccgcaaguucuugcugugccuacuagu

SEQ ID NO: 8 - DNA, 86 bp, Artificial Sequence
  Synthetic Polynucleotide
cucgagugagcgcgcgccugggagaugcuguugacuguaaagccacagaugggucaacagcaucucccaggcgcaugccuacuagu

SEQ ID NO: 9 - DNA, 86 bp, Artificial Sequence
  Synthetic Polynucleotide
cucgagugagcgacgccugggagaugcuguugaacuguaaagccacagauggguucaacagcaucucccaggcgcugccuacuagu

SEQ ID NO: 10 - DNA, 86 bp, Artificial Sequence
  Synthetic Polynucleotide
cucgagugagcgaccugggagaugcuguugaacacuguaaagccacagauggguguucaacagcaucucccaggcugccuacuagu

SEQ ID NO: 11 - DNA, 86 bp, Artificial Sequence
  Synthetic Polynucleotide
cucgagugagcgacuguugaacagggugugauuacuguaaagccacagauggguaaucacacccuguucaacagcugccuacuagu

SEQ ID NO: 12 - DNA, 86 bp, Artificial Sequence
  Synthetic Polynucleotide
cucgagugagcgagcuauuucauuggccgcaucucuguaaagccacagaugggagaugcggccaaugaaauagccugccuacuagu

SEQ ID NO: 13 - DNA, 86 bp, Artificial Sequence
  Synthetic Polynucleotide
cucgagugagcgacucacgaagguuggaauaucucuguaaagccacagaugggagauauuccaaccuucgugaggugccuacuagu

SEQ ID NO: 14 - DNA, 86 bp, Artificial Sequence
  Synthetic Polynucleotide
cucgagugagcgcccggcagcacauggagaaugacuguaaagccacagaugggucauucuccaugugcugccggaugccuacuagu

SEQ ID NO: 15 - DNA, 86 bp, Artificial Sequence
  Synthetic Polynucleotide
cucgagugagcgagcagcacauggagaaugagaucuguaaagccacagaugggaucucauucuccaugugcugccugccuacuagu

SEQ ID NO: 16 - DNA, 86 bp, Artificial Sequence
  Synthetic Polynucleotide
cucgagugagcgagagaaugagauggcccauuaucuguaaagccacagaugggauaaugggccaucucauucuccugccuacuagu

SEQ ID NO: 17 - DNA, 86 bp, Artificial Sequence
  Synthetic Polynucleotide
cucgagugagcgacccauuaugccugugacuguucuguaaagccacagaugggaacagucacaggcauaaugggcugccuacuagu

SEQ ID NO: 18 - DNA, 86 bp, Artificial Sequence
  Synthetic Polynucleotide
cucgagugagcgaagccaccaaagucccacuugucuguaaagccacagaugggacaagugggacuuugguggcucugccuacuagu

SEQ ID NO: 19 - DNA, 86 bp, Artificial Sequence
  Synthetic Polynucleotide
cucgagugagcgcgccaccaaagucccacuuguacuguaaagccacagauggguacaagugggacuuugguggcuugccuacuagu

SEQ ID NO: 20 - DNA, 86 bp, Artificial Sequence
  Synthetic Polynucleotide
cucgagugagcgccccugcuguaguugcuccauucuguaaagccacagaugggaauggagcaacuacagcagggaugccuacuagu

SEQ ID NO: 21 - DNA, 86 bp, Artificial Sequence
  Synthetic Polynucleotide
cucgagugagcgaccugcuguaguugcuccauuccuguaaagccacagauggggaauggagcaacuacagcagggugccuacuagu

SEQ ID NO: 22 - DNA, 86 bp, Artificial Sequence
  Synthetic Polynucleotide
cucgagugagcgacugcuguaguugcuccauucacuguaaagccacagaugggugaauggagcaacuacagcaggugccuacuagu

SEQ ID NO: 23 - DNA, 86 bp, Artificial Sequence
  Synthetic Polynucleotide
cucgagugagcgaugcuguaguugcuccauucaacuguaaagccacagauggguugaauggagcaacuacagcagugccuacuagu

SEQ ID NO: 24 - DNA, 86 bp, Artificial Sequence
  Synthetic Polynucleotide
cucgagugagcgacuguaguugcuccauucaaaucuguaaagccacagaugggauuugaauggagcaacuacagcugccuacuagu

SEQ ID NO: 25 - DNA, 86 bp, Artificial Sequence
  Synthetic Polynucleotide
cucgagugagcgaggcagauaagagcagagaucucuguaaagccacagaugggagaucucugcucuuaucugccgugccuacuagu

SEQ ID NO: 26 - DNA, 22 bp, Artificial Sequence
  Synthetic Polynucleotide
acgcggucgucccucuagaaca

SEQ ID NO: 27 - DNA, 22 bp, Artificial Sequence
  Synthetic Polynucleotide
ucggguuucuacuauaacaucu

SEQ ID NO: 28 - DNA, 22 bp, Artificial Sequence
  Synthetic Polynucleotide
cacgaaacuucuuguuauaaua

SEQ ID NO: 29 - DNA, 22 bp, Artificial Sequence
  Synthetic Polynucleotide
ccuccgucgugaaauagguucu

SEQ ID NO: 30 - DNA, 22 bp, Artificial Sequence
  Synthetic Polynucleotide
cuggagaccgguacaucuguuu

SEQ ID NO: 31 - DNA, 22 bp, Artificial Sequence
  Synthetic Polynucleotide
ggagaccgguacaucuguuuaa

SEQ ID NO: 32 - DNA, 22 bp, Artificial Sequence
  Synthetic Polynucleotide
gucguucuugaacgccuagaaa

SEQ ID NO: 33 - DNA, 22 bp, Artificial Sequence
  Synthetic Polynucleotide
acgcggacccucuacgacaacu

SEQ ID NO: 34 - DNA, 22 bp, Artificial Sequence
  Synthetic Polynucleotide
cgcggacccucuacgacaacuu

SEQ ID NO: 35 - DNA, 22 bp, Artificial Sequence
  Synthetic Polynucleotide
cggacccucuacgacaacuugu

SEQ ID NO: 36 - DNA, 22 bp, Artificial Sequence
  Synthetic Polynucleotide
cgacaacuugucccacacuaau

SEQ ID NO: 37 - DNA, 22 bp, Artificial Sequence
  Synthetic Polynucleotide
ccgauaaaguaaccggcguaga

SEQ ID NO: 38 - DNA, 22 bp, Artificial Sequence
  Synthetic Polynucleotide
ggagugcuuccaaccuuauaga

SEQ ID NO: 39 - DNA, 22 bp, Artificial Sequence
  Synthetic Polynucleotide
aggccgucguguaccucuuacu

SEQ ID NO: 40 - DNA, 22 bp, Artificial Sequence
  Synthetic Polynucleotide
ccgucguguaccucuuacucua

SEQ ID NO: 41 - DNA, 22 bp, Artificial Sequence
  Synthetic Polynucleotide
ccucuuacucuaccggguaaua

SEQ ID NO: 42 - DNA, 22 bp, Artificial Sequence
  Synthetic Polynucleotide
cggguaauacggacacugacaa

SEQ ID NO: 43 - DNA, 22 bp, Artificial Sequence
  Synthetic Polynucleotide
cucggugguuucagggugaaca

SEQ ID NO: 44 - DNA, 22 bp, Artificial Sequence
  Synthetic Polynucleotide
ucggugguuucagggugaacau

SEQ ID NO: 45 - DNA, 22 bp, Artificial Sequence
  Synthetic Polynucleotide
agggacgacaucaacgagguaa

SEQ ID NO: 46 - DNA, 22 bp, Artificial Sequence
  Synthetic Polynucleotide
gggacgacaucaacgagguaag

SEQ ID NO: 47 - DNA, 22 bp, Artificial Sequence
  Synthetic Polynucleotide
ggacgacaucaacgagguaagu

SEQ ID NO: 48 - DNA, 22 bp, Artificial Sequence
  Synthetic Polynucleotide
gacgacaucaacgagguaaguu

SEQ ID NO: 49 - DNA, 22 bp, Artificial Sequence
  Synthetic Polynucleotide
cgacaucaacgagguaaguuua

SEQ ID NO: 50 - DNA, 22 bp, Artificial Sequence
  Synthetic Polynucleotide
gccgucuauucucgucucuaga

SEQ ID NO: 51 - DNA, 2246 bp, Artificial Sequence
  Synthetic Polynucleotide
ctcgaggctagcatgccgagtcccagaccggtattgttgcggggggcccgcgctgctcttctcttgttgcttccaccgaggttgctggcccggccctcactgctcttgcgccgctcactgagtgccgccagttgccctccaatttccctgcctgcagcagcatcaagatcatctatggatggggccggtgccgaggaagtactcgcccccctcagacttgccgtaaggcaacaaggggacctggtaaggaagcttaaagaggacaaagccccgcaggtagacgttgataaggccgttgctgagctcaaggcccgaaagcgagttttggaggcaaaggaactcgcattgcaaccgaaggatgacatagtagatagggcgaagatggaggacacgctcaagaggcgatttttctacgaccaggcgtttgctatctatgggggggtttctggtctttatgattttggcccagtcggatgtgcgctgaagaacaacatcatccagacttggcgccagcattttatacaagaggagcaaatccttgagattgactgcacaatgttgacacccgagcctgtactcaaaacctcaggtcatgttgataagttcgccgattttatggtaaaggatgtaaagaatggcgagtgttttcgagcagatcatttgttgaaggcccacctccagaagttgatgtctgacaaaaaatgctccgtcgaaaaaaagtctgagatggagtccgtcttggcccaattggataactacggccaacaagaactggctgacttgttcgttaattacaatgtcaagagtcctatcacaggcaatgacctgtcccccccggtgtcattcaatcttatgttcaagacttttattgggcctggaggaaatatgccgggttatttgagacctgagaccgctcaaggcatttttttgaattttaagagattgctcgaatttaaccaaggcaaattgccttttgctgccgcacaaatcggaaactccttcagaaacgaaatttctccgaggagtggtctcataagagtgagagagttcactatggcagagattgagcatttcgtcgatccttctgaaaaggaccaccctaagtttcagaatgtagccgacttgcacctttacctctattctgcgaaagctcaagtctcaggccaaagtgcacgaaagatgaggcttggcgacgccgtagaacagggcgtgataaataataccgtcctgggatattttattggccggatttacctttatctcaccaaggtgggtatatcacccgacaaactgcggttccgccaacacatggaaaacgaaatggcacattatgcttgcgattgctgggatgctgaatctaagacatcatatggttggatcgagatagtgggatgcgccgaccgatcatgttatgatctgagttgccatgcccgcgcgacgaaggtgcccctggttgctgaaaaacctctgaaagaacctaagacggttaacgtcgttcagtttgagccgtccaagggggcaattggtaaagcatacaagaaggatgcgaaactggtaatggagtatttggctatctgtgacgaatgttatattactgagatggagatgctgctgaatgaaaaaggagaatttactattgaaacggagggtaaaacatttcaactgaccaaagatatgatcaacgtaaaaaggttccaaaaaactctgtatgtagaagaggtagtgccgaatgtaatagaaccctcctttggtcttggcagaattatgtacaccgtttttgaacatactttccatgtccgcgagggcgatgaacagaggacctttttttccttcccggcagtcgtggccccttttaagtgcagtgtcctccccttgtcccaaaatcaggagttcatgccattcgtgaaggaactttcagaggccctcacgcgacatggagtatctcacaaagtcgatgactccagtggctcaattggccgccgctatgcaagaacggacgagattggcgtagcattcggcgtaaccattgatttcgacaccgtcaacaagactccccatacggctacactgcgggatcgagactcaatgcgacaaataagggccgagatttctgagctgccatccatcgtccaggacctggcgaacgggaatataacctgggcagatgtcgaagcacgctaccccctgtttgaaggtcaggagacgggaaaaaaagagaccatcgaggagtgaggtaccgcggccgc

SEQ ID NO: 52 - DNA, 2220 bp, Artificial Sequence
  Synthetic Polynucleotide
atgccgtcccctaggcccgtattgttgcgcggtgcgcgggccgcactcttgctccttttgccacctaggttgcttgctcgaccttcccttttgctcagacggtctctgtcagctgctagttgtccaccgatctccctgccagccgcggccagtcgatcttccatggacggggcaggggcggaggaggtgcttgccccacttagattggcagttaggcaacagggtgatttggtgaggaaattgaaagaggacaaggcaccgcaagtagacgtcgacaaagctgttgcagagctgaaggcaagaaaacgggtcttggaggccaaggaacttgcattgcagccgaaggacgacatcgttgatcgcgccaagatggaggacactctcaagaggaggttcttctatgatcaggctttcgcaatttatggaggagtaagtggtctgtatgacttcggacctgtcggatgtgccctcaagaacaacataattcagacatggagacaacacttcatccaggaagaacagatcctcgaaattgactgcacaatgctgacgccggagcccgttcttaagacgagtggtcatgtagataagttcgctgatttcatggtcaaggacgtcaaaaacggagaatgctttagagccgatcatctcctgaaagctcaccttcaaaaactgatgagcgataagaagtgttcagtggagaaaaaatctgagatggagtccgttctggctcagctggataattatggtcaacaggagcttgcagatctgtttgtgaactacaatgtgaagtcaccaataactggaaatgacctctctccccccgtctcattcaatctgatgttcaaaaccttcattggccccggtggcaatatgccgggatatctccggcccgaaacagcccaggggatctttctcaatttcaaaaggcttcttgaatttaatcagggtaaacttcctttcgcagcggcccagattggaaactcctttcgaaatgaaattagtccccgctcagggctgattagagtccgcgagtttacaatggctgaaattgaacacttcgtagacccttctgagaaggaccatccgaagttccagaacgtggccgatctccacctgtatttgtactccgccaaagcccaggtcagtgggcagagcgctcggaaaatgcgcctgggggatgcggtggagcagggcgtgataaacaatactgtcttggggtattttattggcaggatatatctttatttgacaaaagtcggtatatccccagacaagctccggtttcgccagcacatggagaatgaaatggctcactacgcatgtgattgctgggacgccgaatcaaagacttcatacggttggatagaaatcgtcggctgcgccgatcgaagctgctacgacctcagttgccacgcccgggcgacgaaagtgcctcttgtggcagaaaagcccttgaaagagcccaaaaccgtcaacgtggtccagttcgaaccctccaaaggggctataggaaaggcatataagaaggatgcaaaattggtaatggagtaccttgcaatctgcgacgaatgttatataaccgagatggaaatgctgctgaatgagaagggtgagtttaccatagaaactgaaggaaaaacattccaactgacgaaggatatgataaacgttaagcgcttccagaaaacgttgtatgttgaagaagttgtccctaacgtgattgagccttcattcggactgggacgaattatgtataccgttttcgagcatacttttcacgtgcgcgagggcgacgaacaacgcacttttttctcattcccggccgttgtcgcgccttttaaatgttctgtattgccacttagtcaaaatcaagaatttatgccctttgtaaaggaactctctgaggcgctgacacggcacggcgtttctcataaggtcgacgattccagcggatcaattggtcgaagatatgcccggacggacgaaatcggggtcgcctttggcgtcaccatcgatttcgacactgtcaacaagacacctcacacagcgactttgcgggaccgcgattccatgagacaaattagagctgagatatctgagctcccctccatcgtacaagatctcgccaatggtaatataacctgggcggatgttgaggcgcgataccctcttttcgaggggcaggagaccggcaaaaaagagacgatcgaggaatga

SEQ ID NO: 53 - DNA, 2220 bp, Artificial Sequence
  Synthetic Polynucleotide
atgccgtcaccgcgacccgtactgttgcgcggcgcacgagcagcgctgttgctgctccttccaccaagattgttggcacgaccatccctgctgttgaggcgctccctttctgctgcgtcttgtcctcctatttcacttcctgcagcggcaagtcgcagttccatggatggtgccggagctgaggaggttctcgcaccactgagactggcggttaggcagcaaggagacctggttcgaaaactgaaggaagataaggctccacaggtagatgtggacaaggctgtggctgaacttaaagcccggaagcgggtgctggaagcaaaggaattggctctgcagccaaaggacgatatagtggaccgagccaagatggaagacacattgaaaagacgcttcttttatgaccaggcgtttgctatatatgggggcgtgtccggtctgtacgacttcgggccagttgggtgcgcacttaagaacaacatcatacagacgtggagacaacacttcatccaggaggagcagattctcgaaatcgactgtacgatgttgactcctgaaccagtattgaaaacctcaggccacgtcgacaagttcgccgacttcatggtgaaagatgttaagaacggagaatgttttcgagcagaccacctccttaaagcccatttgcaaaaactgatgagtgacaaaaagtgctctgtggagaagaagtcagaaatggaatcagtgttggcacaactcgataactacggccaacaggaacttgctgatcttttcgtaaactataacgttaagtcacccattacgggaaatgatcttagtccgcctgttagcttcaatttgatgtttaaaacgtttataggtcccggggggaacatgccagggtatttgcggccagaaactgcgcagggtattttccttaactttaaacgattgttggagttcaatcagggaaaactcccattcgctgccgcacaaatcggcaacagctttagaaatgagatctcacctcgctccgggcttattagagttcgagagtttaccatggccgaaatcgaacacttcgtcgatccctccgaaaaggatcacccaaaatttcagaacgtcgcagacctccacctgtacctgtactcagctaaagcgcaggttagcggccaatccgctcgaaaaatgaggctcggagacgccgtcgagcagggtgtgattaataatacggttctggggtatttcatcggtcgaatatatctgtaccttactaaagttggtatttctcctgacaaattgcgcttccgccaacatatggaaaatgagatggctcattatgcgtgtgattgctgggacgcagagtctaaaacaagttacggatggatagagattgtagggtgcgcggaccggtcttgctatgacctcagttgccatgcgagagcgaccaaagtgcccttggtggctgagaagccccttaaagaacccaaaacggtcaatgtagtccaattcgagccctccaaaggagcgattgggaaagcgtacaaaaaggacgctaagctggtcatggaatatctggctatttgtgacgaatgttacataacggaaatggagatgcttcttaatgaaaagggggaattcacgattgagaccgaggggaaaacatttcagctcacaaaggatatgatcaacgtgaaacggtttcagaagacgctgtacgtcgaagaggttgttccgaacgtaattgagcctagttttggactcgggaggatcatgtataccgtcttcgaacacacctttcacgtccgagagggagacgagcagaggacgttcttttctttccccgccgtcgttgctccattcaagtgctcagtgctccctcttagtcagaatcaggaattcatgccatttgtgaaggaattgagcgaggcactcacaagacacggcgtcagtcataaagttgatgacagttccggtagcataggccggcgatacgcgaggacagatgaaattggagttgcatttggagtcacgattgatttcgatacggttaacaagaccccgcacactgcaacactcagagacagggatagcatgcggcagatacgcgctgaaattagcgagttgcccagtatagtacaggatcttgctaatgggaatattacatgggccgatgtggaggcacggtatccgctgttcgaagggcaagagacagggaaaaaagaaaccatcgaagaatga

SEQ ID NO: 54 - DNA, 2220 bp, Artificial Sequence
  Synthetic Polynucleotide
atgccgagtcccagaccggtattgttgcggggggcccgcgctgctcttctcttgttgcttccaccgaggttgctggcccggccctcactgctcttgcgccgctcactgagtgccgccagttgccctccaatttccctgcctgcagcagcatcaagatcatctatggatggggccggtgccgaggaagtactcgcccccctcagacttgccgtaaggcaacaaggggacctggtaaggaagcttaaagaggacaaagccccgcaggtagacgttgataaggccgttgctgagctcaaggcccgaaagcgagttttggaggcaaaggaactcgcattgcaaccgaaggatgacatagtagatagggcgaagatggaggacacgctcaagaggcgatttttctacgaccaggcgtttgctatctatgggggggtttctggtctttatgattttggcccagtcggatgtgcgctgaagaacaacatcatccagacttggcgccagcattttatacaagaggagcaaatccttgagattgactgcacaatgttgacacccgagcctgtactcaagacctcaggtcatgtagacaaattcgccgattttatggtaaaggatgtaaagaatggcgagtgttttcgagcagatcatttgttgaaggcccacctccagaagttgatgtctgacaaaaaatgctccgtcgaaaaaaagtctgagatggagtccgtcttggcccaattggataactacggccaacaagaactggctgacttgttcgttaattacaatgtcaagagtcctatcacaggcaatgacctgtcccccccggtgtcattcaatcttatgttcaagacttttattgggcctggaggaaatatgccgggttatttgagacctgagaccgctcaaggcatttttttgaattttaagagattgctcgaatttaaccaaggcaaattgccttttgcggccgcacaaatcggaaactccttcagaaacgaaatttctccgaggagtggtctcataagagtgagagaattcactatggcagagattgagcatttcgtcgatccttctgaaaaggaccaccctaagtttcagaatgtagccgacttgcacctttacctctattctgcgaaagctcaagtctcaggccaaagtgcacgaaagatgaggcttggcgacgccgtagaacagggcgtgataaataataccgtcctgggatattttattggccggatttacctttatctcaccaaggtgggtatatcacccgacaaactgcggttccgccaacacatggaaaacgaaatggcacattatgcttgcgactgttgggatgctgaatctaagacatcatatggttggatcgagatagtgggatgcgccgaccgatcatgttatgatctgagttgccatgcccgcgcgacgaaggtgcccctggttgctgaaaaacctctgaaagaacctaagacggttaacgtcgttcagtttgagccgtccaagggggcaattggtaaagcatacaagaaggatgcgaaactggtaatggagtatttggctatctgtgacgaatgttatattactgagatggagatgctgctgaatgaaaaaggagaatttactattgaaacggagggtaaaacatttcaactgaccaaagatatgatcaacgtaaaaaggttccaaaaaactctgtatgtagaagaggtagtgccgaatgtaatagaaccctcctttggtcttggcagaattatgtacaccgtttttgaacatactttccatgtccgcgagggcgatgaacagaggacctttttttccttcccggcagtcgtggccccttttaagtgcagtgtcctccccttgtcccaaaatcaggagttcatgccattcgtgaaggaactttcagaggccctcacgcgacatggagtatctcacaaagtcgatgactccagtggctcaattggccgccgctatgcaagaacggacgagattggcgtagcattcggcgtaaccattgatttcgacaccgtcaacaagactccccatacggctacactgcgggatcgagactcaatgcgacaaataagggccgagatttctgagctgccatccatcgtccaggacctggcgaacgggaatataacctgggcagatgtcgaagcacgctaccccctgtttgaaggtcaggagacgggaaaaaaagagaccatcgaggagtga

SEQ ID NO: 55 - DNA, 2220 bp, Artificial Sequence
  Synthetic Polynucleotide
atgccttctccgagacctgtgttgttgcgaggggctcgcgctgctttgctcctcttgctcccgcctcgcctgcttgctcgcccctccttgttgcttcgcaggtccctgtctgctgctagttgccctccaattagtttgcccgcagccgcgtcaaggagttcaatggacggggctggcgcagaagaggtccttgccccgttgaggctggctgtgcgacagcaaggtgaccttgtgagaaagctgaaggaagacaaagctccgcaagtcgacgtcgacaaggcagtcgctgagttgaaggcccgaaaacgggtacttgaagccaaggaattggctctccagccaaaagacgacatagtagatcgagcgaagatggaagatactctcaagcggaggttcttttatgaccaggcatttgcaatctacgggggtgtttcaggtctttatgattttggtcccgttggttgcgcccttaaaaataatatcatacaaacatggagacaacacttcattcaagaagagcaaattctggaaatcgactgcaccatgctcacaccagagccagtgttgaagacaagtggtcatgtggacaaatttgctgatttcatggtgaaagatgtcaaaaatggagagtgctttagggcggatcatctcttgaaagcccatttgcaaaaactgatgtcagacaaaaaatgcagcgtagaaaaaaagagtgaaatggagagcgtattggctcagcttgataattatggacaacaggagctcgccgatcttttcgtcaactataatgtcaagtcacccattactggcaacgacttgagccctcccgtaagtttcaatctcatgtttaaaaccttcatcggcccagggggcaatatgcctggatacttgcggcccgaaaccgcccaaggtattttcctcaattttaagaggcttttggaattcaatcagggtaagctcccttttgccgccgcgcaaattggcaatagtttcaggaatgaaattagccctcgcagtggtttgattagggtcagggagtttacgatggcagagatagagcatttcgtggacccgagtgaaaaagaccacccgaaatttcagaatgtagcggacctccacctctatctgtattcagcgaaagcgcaggttagcgggcaaagcgcgcggaagatgcggcttggcgatgctgtggaacaaggagtcataaataacaccgtattgggttattttatagggcgaatttatctctatcttacaaaggtaggcatttcaccggataaacttaggttccgacaacacatggagaatgaaatggcgcattacgcctgtgattgttgggatgctgagtctaaaacctcctatggatggattgagattgtcggatgtgcagaccgatcatgttacgacctgagttgccacgcacgagcaaccaaagtaccactcgtggcggaaaaaccgttgaaagagcccaagaccgttaatgtggtccaattcgaacccagtaagggagccattggaaaggcatataaaaaggatgctaaactggtcatggaatatctggcgatctgcgacgaatgttatattacagaaatggaaatgcttttgaacgaaaaaggcgagtttaccatagagaccgaggggaaaacttttcagttgactaaggacatgataaacgtcaaacggtttcagaaaactctctatgttgaggaagttgtcccgaacgttatagagcccagcttcggcctgggtcggattatgtatactgtatttgagcacacattccatgtacgagagggggatgaacagcggactttctttagtttcccggccgtagtagccccctttaagtgttcagtcctccctctctcacagaatcaggagtttatgccattcgtaaaagagttgagtgaagctctgactcggcatggggttagccacaaggtcgacgatagcagcgggagcatcggaaggagatatgcccgaacagacgagatcggtgtggcgttcggtgttactattgattttgatactgtgaacaagaccccacacactgctaccctgagggaccgggattcaatgagacaaatcagggccgaaatctccgagctgccaagtattgtccaagacctcgcaaacgggaatatcacctgggctgacgtagaagcgcgatatcccctgttcgagggacaagaaactggaaagaaggaaactattgaagaatga

SEQ ID NO: 56 - DNA, 2220 bp, Artificial Sequence
  Synthetic Polynucleotide
atgcccagtccccggcctgtccttctgagaggggctcgggccgcactcttgttgctgttgccccccaggctgctggcgcgaccaagcttgctgctgcgaagatccctgtccgccgcgtcctgtcctcctatatcactccccgcggcggcgagtaggagctccatggatggtgcaggcgcagaagaagtactcgccccactcaggcttgctgtacggcaacaaggggatttggtcaggaaattgaaagaagacaaagcaccccaagttgacgtcgataaggccgttgccgaacttaaagccagaaaacgagttcttgaggcaaaggaacttgccctccagcctaaagatgacattgttgaccgggcaaaaatggaggacacactcaagcggagatttttttacgatcaagccttcgcaatttatggtggcgttagcggcctctatgacttcggtcccgtcgggtgtgctctgaagaacaatatcatccagacctggcggcaacactttatccaagaggaacagattttggaaatagactgcacgatgctcaccccagaacctgtgttgaagacttcagggcatgtcgataaattcgcggatttcatggtgaaggacgttaagaatggcgaatgtttccgagctgatcaccttcttaaggcgcatttgcaaaaactcatgtctgataagaaatgttctgtggaaaaaaaatctgagatggaatcagttcttgcacagttggataactacggtcaacaagaactggcagatttgtttgttaattacaacgtaaaaagccctatcaccgggaatgatttgtcccctcccgtcagttttaaccttatgttcaaaacctttatcggtcccggtggcaacatgccaggttatctccgccctgaaacggctcaagggattttcctgaatttcaaaaggttgttggagtttaatcaggggaaacttcccttcgccgccgctcaaataggtaacagttttcgcaacgagatctctccccggagtggcttgattcgcgtgcgcgaatttactatggccgaaatagaacattttgttgacccatccgagaaggaccaccctaagtttcagaacgtagccgatttgcatttgtacttgtatagtgcaaaagcgcaggtaagcggacaaagtgcgcgaaaaatgcggctgggtgatgctgtagagcaaggcgtcattaataatactgtactgggctactttataggaagaatttacctttacctcaccaaggttggaattagccccgacaagttgcgctttcgccagcacatggaaaacgagatggcacactatgcatgcgattgttgggacgcagaaagcaagacttcctatggatggatcgaaatagtcgggtgcgctgataggtcatgctatgatttgtcttgtcatgcaagggcgacgaaggtccctttggttgcggaaaaaccccttaaggaaccaaagacggtgaacgtagttcagtttgaaccctccaaaggtgctatagggaaggcatacaaaaaggatgccaagttggtaatggaatacctggcaatatgtgacgagtgctacattacggaaatggagatgctgctgaatgaaaaaggtgagtttacaatcgagactgaagggaaaacttttcagctcactaaggacatgatcaatgtaaagagatttcaaaaaacgctttatgtggaggaagtggtaccgaatgtgattgaaccttcttttgggcttggtcgaattatgtacacagtttttgaacacacttttcacgtccgggaaggggatgaacaacggacgtttttctcttttcctgccgtagttgccccttttaagtgtagtgtactccctctctcccaaaatcaagagttcatgccgtttgtaaaagaattgagtgaggcccttacacgacacggcgttagtcacaaggtggacgattcatcaggtagcatcgggcggcgatatgcgagaaccgacgagatcggtgtagcttttggggtgactatagattttgacacagttaataaaacgccccatacggccacactgcgcgaccgagattccatgcgacagataagagcagaaatctctgaactgccaagtatcgttcaggatctcgccaatggaaacatcacgtgggcagacgtagaagcccgatatccgttgtttgaaggacaagaaacgggcaagaaggagaccatcgaagagtga

SEQ ID NO: 57 - DNA, 2220 bp, Artificial Sequence
  Synthetic Polynucleotide
atgccgtccccgcggccagtcttgcttcggggtgcccgggcggccctgttgcttctcctgccgcctcggctgttggcccgcccgagtctgctcctccgacgaagccttagcgccgcgtcatgtccccccatcagcctcccagctgcggcaagtcgctcatccatggatggagccggcgcagaagaagttcttgccccactgcgcttggccgtacggcaacaaggggatctggtcagaaagctcaaggaggacaaggcaccacaagtggatgtcgataaggctgttgccgagctcaaagctagaaagcgcgttttggaagcgaaggaactggctctgcagccaaaggacgacattgttgacagagcaaaaatggaagacacattgaagaggcggtttttctatgaccaggcgtttgctatttatgggggggtatccggattgtacgacttcggtccggtagggtgcgctctcaaaaataatatcatccagacttggcggcaacatttcatacaagaagagcagattctcgaaattgattgtacaatgttgacgcctgaacccgtactgaaaacgtccggtcatgttgataaattcgctgattttatggtgaaggatgtaaaaaatggcgaatgctttcgagccgaccacttgctgaaggcccatttgcaaaagcttatgagtgacaagaaatgttcagtcgagaaaaagagtgagatggagtcagtcttggcccaactggacaattatggacagcaggagctcgccgacctctttgtcaactataatgtcaagagtccgattactggaaatgacctcagtccacctgtcagtttcaatttgatgttcaaaacctttattgggccgggcgggaatatgccaggctatctgagacccgagactgcacaaggcatatttttgaactttaagcgcctgctggagtttaaccaaggcaagcttcctttcgcagcagcgcaaattggtaatagcttcaggaatgagatcagtcctagatcaggccttatccgagtaagggagtttacgatggccgaaatcgaacactttgtcgacccctccgaaaaggatcatccaaaatttcagaatgtggcggatttgcacctttacctgtactcagctaaagcccaagtatcaggccagtccgcacggaaaatgaggcttggtgatgcggtcgagcaaggtgtaatcaataatacagtcttgggctactttatcggacgcatttatctttatctgacaaaagtgggtatctctcctgacaaactcagattccgccaacatatggaaaatgagatggcccattacgcctgcgattgctgggatgctgagagtaagacaagctatgggtggatagagattgtggggtgcgcggatcgatcctgttacgatttgtcatgtcatgcacgcgcgacaaaggtgcccttggtcgccgagaagccccttaaagaaccgaagaccgttaatgtggtgcagttcgagccctcaaagggggcaatcggaaaagcatacaaaaaagatgcgaaattggtcatggaatatttggctatttgcgacgaatgttacataacagaaatggagatgcttctcaacgaaaaaggtgaatttacaatagagacagagggcaagacatttcaactcactaaggatatgatcaacgtaaaaaggtttcagaaaacgctctatgtagaggaggttgtcccgaacgtaatagaaccctcattcgggctgggacgcataatgtacacggtgttcgagcacactttccacgtaagggagggagatgaacagcgaaccttcttctctttccctgccgtggtggcaccattcaagtgctctgtattgccccttagtcaaaaccaggaatttatgccctttgttaaagaattgtccgaggctttgacacgacacggagtgtcccataaagtggacgactcaagtggatctataggtaggagatatgcacgcactgatgagattggagtcgcgtttggggtgacgatagattttgatacagtaaataaaacgccacatacagctacgctcagggaccgcgatagcatgcgacaaatccgggcggaaatatccgaactgccaagcatagtacaagatcttgcaaatggaaacataacctgggccgacgttgaggcccgctacccactttttgaaggccaagaaacaggcaaaaaggagacaatagaggagtga

SEQ ID NO: 58 - DNA, 21 bp, Artificial Sequence
  Synthetic Polynucleotide
  misc_feature: Forward PCR Primer
gcattgccaaagtagtactgc

SEQ ID NO: 59 - DNA, 24 bp, Artificial Sequence
  Synthetic Polynucleotide
  misc_feature: Reverse PCR Primer
cctgactctgatcagtccagatcg

SEQ ID NO: 60 - DNA, 130 bp, Artificial Sequence
  Synthetic Polynucleotide
  misc_feature: ssODN donor
agtttacttgtaacaggctttgttttattggaagcacattgtcttacttgtaatagactggtttatttaattttatagatacttgagaccggggattttcttgaatttcaaacgacttttggaattcaac

SEQ ID NO: 61 - DNA, 20 bp, Artificial Sequence
  Synthetic Polynucleotide
  misc_feature: sgRNA
aaaattccctgtgcagtttc

SEQ ID NO: 62 - DNA, 20 bp, Artificial Sequence
  Synthetic Polynucleotide
  misc_feature: sgRNA 1340
tcagaaatgagatctcacct

SEQ ID NO: 63 - DNA, 21 bp, Artificial Sequence
  Synthetic Polynucleotide
  misc_feature: Primers HuEx8F0_F
cataacatcacgcgtggttcc

SEQ ID NO: 64 - DNA, 20 bp, Artificial Sequence
  Synthetic Polynucleotide
  misc_feature: HuEx8R0_R
caagtgtggcggtttccatc

SEQ ID NO: 65 - DNA, 24 bp, Artificial Sequence
  Synthetic Polynucleotide
  misc_feature: delETAQF0_F
ggccataagcataattttactgtg

SEQ ID NO: 66 - DNA, 24 bp, Artificial Sequence
  Synthetic Polynucleotide
  misc_feature: Delta- ETAGF0_R
tacaacagaaacaaactgtggtca

SEQ ID NO: 67 - DNA, 20 bp, Artificial Sequence
  Synthetic Polynucleotide
  misc_feature: Primers GARS2F
ctcccaccactggcaatgac

SEQ ID NO: 68 - DNA, 19 bp, Artificial Sequence
  Synthetic Polynucleotide
  misc_feature: Primer GARS2R
ctcactcagcagcagctcc

SEQ ID NO: 69 - DNA, 2220 bp, Homo Sapiens
  misc_feature: Human coding sequence
atgccctctccgcgtccagtgctgcttagaggtgctcgcgccgctctgctgctgctgctgccgccccggctcttagcccgaccctcgctcctgctccgccggtccctcagcgcggcctcctgccccccgatctccttgcccgccgccgcctcccggagcagcatggacggcgcgggggctgaggaggtgctggcacctctgaggctagcagtgcgccagcagggagatcttgtgcgaaaactcaaagaagataaagcaccccaagtagacgtagacaaagcagtggctgagctcaaagcccgcaagagggttctggaagcaaaggagctggcgttacagcccaaagatgatattgtagaccgagcaaaaatggaagataccctgaagaggaggtttttctatgatcaagcttttgctatttatggaggtgttagtggtctgtatgactttgggccagttggctgtgctttgaagaacaatattattcagacctggaggcagcactttatccaagaggaacagatcctggagatcgattgcaccatgctcacccctgagccagttttaaagacctctggccatgtagacaaatttgctgacttcatggtgaaagacgtaaaaaatggagaatgttttcgtgctgaccatctattaaaagctcatttacagaaattgatgtctgataagaagtgttctgtcgaaaagaaatcagaaatggaaagtgttttggcccagcttgataactatggacagcaagaacttgcggatctttttgtgaactataatgtaaaatctcccattactggaaatgatctatcccctccagtgtcttttaacttaatgttcaagactttcattgggcctggaggaaacatgcctgggtacttgagaccagaaactgcacaggggattttcttgaatttcaaacgacttttggagttcaaccaaggaaagttgccttttgctgctgcccagattggaaattcttttagaaatgagatctcccctcgatctggactgatcagagtcagagaattcacaatggcagaaattgagcactttgtagatcccagtgagaaagaccaccccaagttccagaatgtggcagaccttcacctttatttgtattcagcaaaagcccaggtcagcggacagtccgctcggaaaatgcgcctgggagatgctgttgaacagggtgtgattaataacacagtattaggctatttcattggccgcatctacctctacctcacgaaggttggaatatctccagataaactccgcttccggcagcacatggagaatgagatggcccattatgcctgtgactgttgggatgcagaatccaaaacatcctacggttggattgagattgttggatgtgctgatcgttcctgttatgacctctcctgtcatgcacgagccaccaaagtcccacttgtagctgagaaacctctgaaagaacccaaaacagtcaatgttgttcagtttgaacccagtaagggagcaattggtaaggcatataagaaggatgcaaaactggtgatggagtatcttgccatttgtgatgagtgctacattacagaaatggagatgctgctgaatgagaaaggggaattcacaattgaaactgaagggaaaacatttcagttaacaaaagacatgatcaatgtgaagagattccagaaaacactatatgtggaagaagttgttccgaatgtaattgaaccttccttcggcctgggtaggatcatgtatacggtatttgaacatacattccatgtacgagaaggagatgaacagagaacattcttcagtttccctgctgtagttgctccattcaaatgttccgtcctcccactgagccaaaaccaggagttcatgccatttgtcaaggaattatcggaagccctgaccaggcatggagtatctcacaaagtagacgattcctctgggtcaatcggaaggcgctatgccaggactgatgagattggcgtggcttttggtgtcaccattgactttgacacagtgaacaagaccccccacactgcaactctgagggaccgtgactcaatgcggcagataagagcagagatctctgagctgcccagcatagtccaagacctagccaatggcaacatcacatgggctgatgtggaggccaggtatcctctgtttgaagggcaagagactggtaaaaaagagacaatcgaggaatga

SEQ ID NO: 70 - DNA, 2190 bp, Mus musculus
  misc_feature: Mouse coding sequence
atgccctgtctgctcccctcgctgctcagagccacccgcgccgcgctgccgctcctgtcgccgccccgagtggtcgcagcgtccgcgtctcagcggctcctcagcgctcccgcgcagcccgccgcctcccggagcagcatggacagcgcggaggagctgctggccccactgcggctagccgtgcgccagcagggagactttgtacggaagttaaaagaagataaagccccacaagtggatgtggacagagcagtagctgagctcaaggcccggaagagggttctggaagcaaaggagctggcattgcagcccaaagatgacatcgtagatagagcaaaaatggaagatacgttgaagaggaggtttttctacgatcaggcttttgctatttatggaggtgtcagtggattgtatgacttcgggccggtaggatgtgctttgaagaacaatatcatccaggcctggaggcagcactttatccaagaggagcagatcctggagattgactgcaccatgctcacccctgagccagttttaaagacctctggccacgtagacaaatttgctgacttcatggtgaaggacgtgaagaacggagagtgcttccgagcagaccacctgttgaaagctcatttacagaaactgatgtcagataagaagtgctctgctgagaagaagtcagagatggaaagtgtcttggcccagcttgataactatggacaacaagaacttgcggatctttttgtgaactataatgtaaaatctcccaccactggcaatgacctgtcccctccggtaccttttaacttaatgttccagaccttcattgggcctggaggaaatatgcctggatatctgagaccagaaactgcacagggaattttcctgaatttcaaacgacttttggaattcaaccaagggaaattgccttttgctgctgcccagattggaaactccttcagaaatgagatctcacctcggtctggactgatccgagtcagggagtttacaatggcagagattgagcactttgtagatcccactgagaaagaccatcccaagttccaaagtgtggccgacctctgcctttatttgtactcagcaaaagcccaggtcactggacagtctgctcggaagatgcgtctgggagatgctgttgaacagggtgtgattaacaactcagtattaggctattttattggccgcatctacctctacctcacgaaggttggaatatctcctgataaactccgcttccggcagcatatggagaatgagatggcccattatgcctgcgactgctgggatgccgagtccaaaacgtcctatggctggattgagattgttggatgtgctgaccgttcctgctacgacctctcctgtcatgctcgagccaccaaagttccactagtagctgagaaacctctgaaagaacccaaaacagttaacgttgtacagtttgagcccaacaagggcgccgtgggcaaggcgtacaagaaggatgcaaagctagtgctggagtatctcagcgcctgtgatgagtgctacatttcagagatggagctgctgctgagtgagaaaggggaattcactattgaaactgaaggaaaaacatttcagttaacgaaagacatggtcagtgtgaagagattccagaaaacactgcatgtggaagaagttgttccgagtgtaattgagccctcctttggcttgggcaggatcatgtataccatattggaacatacattccatgtccgagagggagatgaacagagaacgttcttcagtttccctgctgtggttgctccattcaaatgttctgtccttccactgagccagaaccaagagtttatgccatttgtcaaggaattatccgaagctctgaccaggaacggcgtgtctcataaagtcgatgactcctctgggtctattggaaggcgctatgcaagaactgatgagattggcgtggctttcggcatcactattgactttgatacagtgaacaagacgccccacactgcaactctgagggaccgagactccatgagacagataagggcagaggtctctgagctgcccaatgtggtccgcgatctggccaacggcaacattacctgggctgatgtggaggccaggtacccactctttgaagggcaagagactggcaagaaggagacagtagaggaatga

SEQ ID NO: 71 - protein, 739 aa, Homo sapiens
  MISC_FEATURE: Human GARS Protein
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 72 - protein, 729 aa, Mus musculus
  MISC_FEATURE: Mouse GARS protein
MPCLLPSLLRATRAALPLLSPPRVVAASASQRLLSAPAQPAASRSSMDSAEELLAPLRLAVRQQGDFVRKLKEDKAPQVDVDRAVAELKARKRVLEAKELALQPKDDIVDRAKMEDTLKRRFFYDQAFAIYGGVSGLYDFGPVGCALKNNIIQAWRQHFIQEEQILEIDCTMLTPEPVLKTSGHVDKFADFMVKDVKNGECFRADHLLKAHLQKLMSDKKCSAEKKSEMESVLAQLDNYGQQELADLFVNYNVKSPTTGNDLSPPVPFNLMFQTFIGPGGNMPGYLRPETAQGIFLNFKRLLEFNQGKLPFAAAQIGNSFRNEISPRSGLIRVREFTMAEIEHFVDPTEKDHPKFQSVADLCLYLYSAKAQVTGQSARKMRLGDAVEQGVINNSVLGYFIGRIYLYLTKVGISPDKLRFRQHMENEMAHYACDCWDAESKTSYGWIEIVGCADRSCYDLSCHARATKVPLVAEKPLKEPKTVNVVQFEPNKGAVGKAYKKDAKLVLEYLSACDECYISEMELLLSEKGEFTIETEGKTFQLTKDMVSVKRFQKTLHVEEVVPSVIEPSFGLGRIMYTILEHTFHVREGDEQRTFFSFPAVVAPFKCSVLPLSQNQEFMPFVKELSEALTRNGVSHKVDDSSGSIGRRYARTDEIGVAFGITIDFDTVNKTPHTATLRDRDSMRQIRAEVSELPNVVRDLANGNITWADVEARYPLFEGQETGKKETVEE

SEQ ID NO: 73 - protein, 24 aa, Homo Sapiens
FIGPGGNMPGYLRPETAQGIFLNF

SEQ ID NO: 74 - protein, 24 aa, D. Rerio
SIGPGGNMQGYLRPETAQGIFLNF

SEQ ID NO: 75 - protein, 24 aa, D. Melanogaster
QIGPTGLVKGFLRPETAQGIFVNF

SEQ ID NO: 76 - protein, 24 aa, C. Elegans
QIGPTGDFKAFLRPETAQGIFVNF

SEQ ID NO: 77 - protein, 24 aa, S. Cerevisiae
AIGPSGQLKGYLRPETAQGQFLNF

SEQ ID NO: 78 - protein, 24 aa, Escherichia coli
GASADSAMDLYLRPETAQGIFVNF

SEQ ID NO: 79 - DNA, 34 bp, Homo Sapiens
gtacttgagaccagaaactgcacaggggattttc

SEQ ID NO: 80 - DNA, 34 bp, Mus Musculus
atatctgagaccagaaactgcacagggaattttc

SEQ ID NO: 81 - DNA, 34 bp, Homo Sapiens
atatttgagaccggaactgtcctggttttttctt

SEQ ID NO: 82 - protein, 13 aa, Artificial Sequence
  Synthetic Polypeptide
MPGYLRPETAQGI

SEQ ID NO: 83 - DNA, 39 bp, Artificial Sequence
  Synthetic Polynucleotide
augccuggguacuugagaccagaaacugcacagggaacu

SEQ ID NO: 84 - DNA, 22 bp, Artificial Sequence
  Synthetic Polynucleotide
acccauggacucuggcccuuaa

SEQ ID NO: 85 - DNA, 38 bp, Artificial Sequence
  Synthetic Polynucleotide
augccuggguacuuagaccggggauuuucuugaauuuc

SEQ ID NO: 86 - protein, 13 aa, Artificial Sequence
  Synthetic Polypeptide
MPGYLRPGIFLNF

SEQ ID NO: 87 - DNA, 86 bp, Artificial Sequence
  Synthetic Polynucleotide
cucgagugagcgaggguacuugagaccggggauucuguaaagccacagaugggaauccccggucucaaguacccaugccuacuagu

SEQ ID NO: 88 - DNA, 86 bp, Artificial Sequence
  Synthetic Polynucleotide
cucgagugagcgaggguacuugagaccggggauucuguaaagccacagaugggaauccucggucucaaguacccaugccuacuagu

SEQ ID NO: 89 - DNA, 86 bp, Artificial Sequence
  Synthetic Polynucleotide
cucgagugagcgagguacuugagaccggggauuucuguaaagccacagaugggaaauccccggucucaaguacccugccuacuagu

SEQ ID NO: 90 - DNA, 86 bp, Artificial Sequence
  Synthetic Polynucleotide
cucgagugagcgaccuggguacuugagaccggggcuguaaagccacagaugggccccggucucaaguacccaggcugccuacuagu

SEQ ID NO: 91 - DNA, 75 bp, Artificial Sequence
  Synthetic Polynucleotide
cucgagugagcgacuggguacuugagaccggggacuguaaagccacagauggguccccggucucaaguacccagg

SEQ ID NO: 92 - DNA, 40 bp, Artificial Sequence
  Synthetic Polynucleotide
augccugggguacuugagaccggggauuuucuugaauuuc

SEQ ID NO: 93 - DNA, 22 bp, Artificial Sequence
  Synthetic Polynucleotide
acccaugaacucuggccccuaa

SEQ ID NO: 94 - DNA, 39 bp, Artificial Sequence
  Synthetic Polynucleotide
augccuggauaucugagaccgggaauuuuccugaauuuc

SEQ ID NO: 95 - DNA, 88 bp, Artificial Sequence
  Synthetic Polynucleotide
cucgagugagcgaggguacuugagaccggggauucuguaaaagccacagauggggaauccccggucucagguacccaugccuacuagu

SEQ ID NO: 96 - DNA, 86 bp, Artificial Sequence
  Synthetic Polynucleotide
cucgagugagcgaggguacuugagaccggggauucuguaaagccacagaugggaauucccggucucaaguacccaugccuacuagu

SEQ ID NO: 97 - DNA, 86 bp, Artificial Sequence
  Synthetic Polynucleotide
cucgagugagcgaggguacuugagaccggggauucuguaaagccacagaugggaauccccggucucagguauccaugccuacuagu

SEQ ID NO: 98 - DNA, 86 bp, Artificial Sequence
  Synthetic Polynucleotide
cucgagugagcgaggguacuugagaccggggauucuguaaagccacagaugggaauucccggucucagguacccaugccuacuagu

SEQ ID NO: 99 - DNA, 86 bp, Artificial Sequence
  Synthetic Polynucleotide
cucgagugagcgaggguacuugagaccggggauucuguaaagccacagaugggaauucccggucucaaguauccaugccuacuagu

SEQ ID NO: 100 - DNA, 86 bp, Artificial Sequence
  Synthetic Polynucleotide
cucgagugagcgagggaaauauggaggaaauaugcuguaaagccacagaugggcauauuuccuccauauuucccaugacuacuagu

SEQ ID NO: 101 - DNA, 87 bp, Artificial Sequence
  Synthetic Polynucleotide
cucgagugagcgacagaccuucauugggaaauaucucuaaagccacagaugggauauuucccaaaugaaggucuggugccuacuagu

SEQ ID NO: 102 - DNA, 86 bp, Artificial Sequence
  Synthetic Polynucleotide
cucgagugagcgacagacguucauugggaaauaucuguaaagccacagaugggauauuucccaaugaacgucuggugccuacuagu

SEQ ID NO: 103 - DNA, 22 bp, Artificial Sequence
  Synthetic Polynucleotide
acgguaaacaguuccuuaauag

SEQ ID NO: 104 - DNA, 86 bp, Artificial Sequence
  Synthetic Polynucleotide
cucgagugagcgacauugggaaauauggaggaaacuguaaagccacagauggguuuccuccauauuucccaaugaugccuacuagu

SEQ ID NO: 105 - protein, 14 aa, Artificial Sequence
  Synthetic Polypeptide
MFQTFIGKYGGNMP

SEQ ID NO: 106 - DNA, 42 bp, Artificial Sequence
  Synthetic Polynucleotide
auguuccagaccuucauugggaaauauggaggaaauaugcct

SEQ ID NO: 107 - DNA, 22 bp, Artificial Seuqence
  Synthetic Polynucleotide
ggucuggaaguaacccuuuaua

SEQ ID NO: 108 - DNA, 42 bp, Artificial Sequence
  Synthetic Polynucleotide
auguuccagaccuucauugggccuggaggaaauaugccugga

SEQ ID NO: 109 - protein, 14 aa, Artificial Sequence
  Synthetic Polypeptide
MFQTFIGPGGNMPG

SEQ ID NO: 110 - protein, 12 aa, Artificial Sequence
  Synthetic Polypeptide
EFMPFVKELSEA

SEQ ID NO: 111 - DNA, 36 bp, Artificial Sequence
  Synthetic Polynucleotide
gaguucaugccauuugucaaggaauuaucggaagcc

SEQ ID NO: 112 - DNA, 22 bp, Artificial Sequence
  Synthetic Polynucleotide
gauaauuccuugacaaauggca

SEQ ID NO: 113 - DNA, 88 bp, Artificial Sequence
  Synthetic Polynucleotide
cucugagugagcgaggcuacuugacaccgggggauucuguaaagccacagaugggaauccccggucucaaguauccaugccuacuagu

SEQ ID NO: 114 - DNA, 36 bp, Artificial Sequence
  Synthetic Polynucleotide
gaguucaugcccuucgugaaagagcucucggaagcc